SEQUENCE LISTING (448 sequences)

SEQ ID NO: 1 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPALFKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 2 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 3 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 4 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 5 - protein, 214 aa, synthetic construct
DIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 613 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 7 - protein, 598 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 8 - protein, 589 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 9 - protein, 589 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 10 - protein, 589 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 895 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 12 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 13 - protein, 218 aa, synthetic construct
QVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 14 - protein, 425 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 15 - protein, 427 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPPLAQKLKSSPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPPLAQKLKSSPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 16 - protein, 901 aa, synthetic construct
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALASGGPALFKSSFPPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 17 - protein, 1365 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGPALFKSSFPPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPALFKSSFPPGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 18 - protein, 426 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSCDLPQTHSLGNRRALILLAQMGRISHFSCLKDRYDFGFPQEVFDGNQFQKAQAISAFHEMIQQTFNLFSTKDSSAAWDETLLDKFYIELFQQLNDLEACVTQEVGVEEIALMNEDSILAVRKYFQRITLYLMGKKYSPCAWEVVRAEIMRSFSFSTNLQKGLRRKDSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 19 - protein, 426 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 20 - protein, 425 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 21 - protein, 553 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSGGGGSGGGGSGGGGSSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 22 - protein, 553 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSGGGGSGGGGSGGGGSSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 23 - protein, 557 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPALFKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHHEPEA

SEQ ID NO: 24 - protein, 559 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPPLAQKLKSSPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPPLAQKLKSSPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHHEPEA

SEQ ID NO: 25 - protein, 591 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPPGGPAGIGALFKSSFPPLAQKLKSSPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPPGGPAGIGALFKSSFPPLAQKLKSSPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHHEPEA

SEQ ID NO: 26 - protein, 444 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGQVQLQESGGGLVQTGGSLRLSCTTSGTIFSGYTMGWYRQAPGEQRELVAVISGGGDTNYADSVKGRFTISRDNTKDTMYLQMNSLKPEDTAVYYCYSREVTPPWKLYWGQGTQVTVSSAAAYPYDVPDYGSHHHHHH

SEQ ID NO: 27 - protein, 444 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGQVQLQESGGGLVQEGGSLRLSCAASERIFSTDVMGWYRQAAEKQRELVAVVSARGTTNYLDAVKGRFTISRDNARNTLTLQMNDLKPEDTASYYCYVRETTSPWRIYWGQGTQVTVSSAAAYPYDVPDYGSHHHHHH

SEQ ID NO: 28 - protein, 445 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGQVQLQESGGGLVQAGGSLRLSCAASGSIFSANAMGWYRQAPGKQRELVAVISSGGSTNYADSVKGRFTISRDNAKNTVYLQMNSLKPEDTAVYYCMYSGSYYYTPNDYWGQGTQVTVSSAAAYPYDVPDYGSHHHHHH

SEQ ID NO: 29 - protein, 553 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGQAPRLLIYSASFRYSGVPSRFSGSGSGTDFTLTISSLQPEDFATYYCQQYYTYPYTFGGGTKVEIKHHHHHH

SEQ ID NO: 30 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGGGTKVEIK

SEQ ID NO: 31 - protein, 906 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSESNIGSNDVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 32 - protein, 910 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSESNIGSNDVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 33 - protein, 906 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSRSNIGENTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 34 - protein, 910 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGENTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 35 - protein, 906 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSRSNIGEETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 36 - protein, 910 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGEETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 37 - protein, 906 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 38 - protein, 910 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 39 - protein, 906 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGSETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 40 - protein, 910 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 41-194 - skipped (154 entries)

SEQ ID NO: 195 - protein, 8 aa, synthetic construct
GPAGLYAQ

SEQ ID NO: 196 - protein, 8 aa, synthetic construct
GPAGMKGL

SEQ ID NO: 197 - protein, 8 aa, synthetic construct
PGGPAGIG

SEQ ID NO: 198 - protein, 8 aa, synthetic construct
ALFKSSFP

SEQ ID NO: 199 - protein, 8 aa, synthetic construct
ALFFSSPP

SEQ ID NO: 200 - protein, 8 aa, synthetic construct
LAQRLRSS

SEQ ID NO: 201 - protein, 8 aa, synthetic construct
LAQKLKSS

SEQ ID NO: 202 - protein, 30 aa, synthetic construct
GALFKSSFPSGGGPAGLYAQGGSGKGGSGK

SEQ ID NO: 203 - protein, 30 aa, synthetic construct
RGSGGGPAGLYAQGSGGGPAGLYAQGGSGK

SEQ ID NO: 204 - protein, 30 aa, synthetic construct
KGGGPAGLYAQGPAGLYAQGPAGLYAQGSR

SEQ ID NO: 205 - protein, 30 aa, synthetic construct
RGGPAGLYAQGGPAGLYAQGGGPAGLYAQK

SEQ ID NO: 206 - protein, 30 aa, synthetic construct
KGGALFKSSFPGGPAGIGPLAQKLKSSGGS

SEQ ID NO: 207 - protein, 30 aa, synthetic construct
SGGPGGPAGIGALFKSSFPLAQKLKSSGGG

SEQ ID NO: 208 - protein, 30 aa, synthetic construct
RGPLAQKLKSSALFKSSFPGGPAGIGGGGK

SEQ ID NO: 209 - protein, 30 aa, synthetic construct
GGGALFKSSFPLAQKLKSSPGGPAGIGGGR

SEQ ID NO: 210 - protein, 30 aa, synthetic construct
RGPGGPAGIGPLAQKLKSSALFKSSFPGGG

SEQ ID NO: 211 - protein, 30 aa, synthetic construct
RGGPLAQKLKSSPGGPAGIGALFKSSFPGK

SEQ ID NO: 212 - protein, 30 aa, synthetic construct
RSGGPAGLYAQALFKSSFPLAQKLKSSGGG

SEQ ID NO: 213 - protein, 30 aa, synthetic construct
GGPLAQKLKSSALFKSSFPGPAGLYAQGGR

SEQ ID NO: 214 - protein, 30 aa, synthetic construct
GGALFKSSFPGPAGLYAQPLAQKLKSSGGK

SEQ ID NO: 215 - protein, 30 aa, synthetic construct
RGGALFKSSFPLAQKLKSSGPAGLYAQGGK

SEQ ID NO: 216 - protein, 30 aa, synthetic construct
RGGGPAGLYAQPLAQKLKSSALFKSSFPGG

SEQ ID NO: 217 - protein, 30 aa, synthetic construct
SGPLAQKLKSSGPAGLYAQALFKSSFPGSK

SEQ ID NO: 218 - protein, 30 aa, synthetic construct
KGGPGGPAGIGPLAQRLRSSALFKSSFPGR

SEQ ID NO: 219 - protein, 30 aa, synthetic construct
KSGPGGPAGIGALFFSSPPLAQKLKSSGGR

SEQ ID NO: 220 - protein, 30 aa, synthetic construct
SGGFPRSGGSFNPRTFGSKRKRRGSRGGGG

SEQ ID NO: 221 - protein, 8 aa, synthetic construct
GPLGLKAQ

SEQ ID NO: 222 - protein, 8 aa, synthetic construct
LPLGLKAQ

SEQ ID NO: 223 - protein, 8 aa, synthetic construct
SPLGLKAQ

SEQ ID NO: 224 - protein, 8 aa, synthetic construct
QPLGLKAQ

SEQ ID NO: 225 - protein, 8 aa, synthetic construct
KPLGLKAQ

SEQ ID NO: 226 - protein, 8 aa, synthetic construct
FPLGLKAQ

SEQ ID NO: 227 - protein, 8 aa, synthetic construct
HPLGLKAQ

SEQ ID NO: 228 - protein, 8 aa, synthetic construct
PPLGLKAQ

SEQ ID NO: 229 - protein, 8 aa, synthetic construct
APLGLKAQ

SEQ ID NO: 230 - protein, 8 aa, synthetic construct
DPLGLKAQ

SEQ ID NO: 231 - protein, 8 aa, synthetic construct
GPHGLKAQ

SEQ ID NO: 232 - protein, 8 aa, synthetic construct
GPSGLKAQ

SEQ ID NO: 233 - protein, 8 aa, synthetic construct
GPQGLKAQ

SEQ ID NO: 234 - protein, 8 aa, synthetic construct
GPPGLKAQ

SEQ ID NO: 235 - protein, 8 aa, synthetic construct
GPEGLKAQ

SEQ ID NO: 236 - protein, 8 aa, synthetic construct
GPFGLKAQ

SEQ ID NO: 237 - protein, 8 aa, synthetic construct
GPRGLKAQ

SEQ ID NO: 238 - protein, 8 aa, synthetic construct
GPGGLKAQ

SEQ ID NO: 239 - protein, 8 aa, synthetic construct
GPAGLKAQ

SEQ ID NO: 240 - protein, 8 aa, synthetic construct
LPAGLKGA

SEQ ID NO: 241 - protein, 8 aa, synthetic construct
GPAGLYAQ

SEQ ID NO: 242 - protein, 8 aa, synthetic construct
GPANLVAQ

SEQ ID NO: 243 - protein, 8 aa, synthetic construct
GPAALVGA

SEQ ID NO: 244 - protein, 8 aa, synthetic construct
GPANLRAQ

SEQ ID NO: 245 - protein, 8 aa, synthetic construct
GPAGLRAQ

SEQ ID NO: 246 - protein, 8 aa, synthetic construct
GPAGLVAQ

SEQ ID NO: 247 - protein, 8 aa, synthetic construct
GPAGLRGA

SEQ ID NO: 248 - protein, 8 aa, synthetic construct
LPAGLVGA

SEQ ID NO: 249 - protein, 8 aa, synthetic construct
GPAGLKGA

SEQ ID NO: 250 - protein, 8 aa, synthetic construct
GPLALKAQ

SEQ ID NO: 251 - protein, 8 aa, synthetic construct
GPLNLKAQ

SEQ ID NO: 252 - protein, 8 aa, synthetic construct
GPLHLKAQ

SEQ ID NO: 253 - protein, 8 aa, synthetic construct
GPLYLKAQ

SEQ ID NO: 254 - protein, 8 aa, synthetic construct
GPLPLKAQ

SEQ ID NO: 255 - protein, 8 aa, synthetic construct
GPLELKAQ

SEQ ID NO: 256 - protein, 8 aa, synthetic construct
GPLRLKAQ

SEQ ID NO: 257 - protein, 8 aa, synthetic construct
GPLLLKAQ

SEQ ID NO: 258 - protein, 8 aa, synthetic construct
GPLSLKAQ

SEQ ID NO: 259 - protein, 8 aa, synthetic construct
GPLGLYAQ

SEQ ID NO: 260 - protein, 8 aa, synthetic construct
GPLGLFAQ

SEQ ID NO: 261 - protein, 8 aa, synthetic construct
GPLGLLAQ

SEQ ID NO: 262 - protein, 8 aa, synthetic construct
GPLGLHAQ

SEQ ID NO: 263 - protein, 8 aa, synthetic construct
GPLGLRAQ

SEQ ID NO: 264 - protein, 8 aa, synthetic construct
GPLGLAAQ

SEQ ID NO: 265 - protein, 8 aa, synthetic construct
GPLGLEAQ

SEQ ID NO: 266 - protein, 8 aa, synthetic construct
GPLGLGAQ

SEQ ID NO: 267 - protein, 8 aa, synthetic construct
GPLGLPAQ

SEQ ID NO: 268 - protein, 8 aa, synthetic construct
GPLGLQAQ

SEQ ID NO: 269 - protein, 8 aa, synthetic construct
GPLGLSAQ

SEQ ID NO: 270 - protein, 8 aa, synthetic construct
GPLGLVAQ

SEQ ID NO: 271 - protein, 8 aa, synthetic construct
GPLGLKLQ

SEQ ID NO: 272 - protein, 8 aa, synthetic construct
GPLGLKFQ

SEQ ID NO: 273 - protein, 8 aa, synthetic construct
GPLGLKEQ

SEQ ID NO: 274 - protein, 8 aa, synthetic construct
GPLGLKKQ

SEQ ID NO: 275 - protein, 8 aa, synthetic construct
GPLGLKQQ

SEQ ID NO: 276 - protein, 8 aa, synthetic construct
GPLGLKSQ

SEQ ID NO: 277 - protein, 8 aa, synthetic construct
GPLGLKGQ

SEQ ID NO: 278 - protein, 8 aa, synthetic construct
GPLGLKHQ

SEQ ID NO: 279 - protein, 8 aa, synthetic construct
GPLGLKPQ

SEQ ID NO: 280 - protein, 8 aa, synthetic construct
GPLGLKAG

SEQ ID NO: 281 - protein, 8 aa, synthetic construct
GPLGLKAF

SEQ ID NO: 282 - protein, 8 aa, synthetic construct
GPLGLKAP

SEQ ID NO: 283 - protein, 8 aa, synthetic construct
GPLGLKAL

SEQ ID NO: 284 - protein, 8 aa, synthetic construct
GPLGLKAE

SEQ ID NO: 285 - protein, 8 aa, synthetic construct
GPLGLKAA

SEQ ID NO: 286 - protein, 8 aa, synthetic construct
GPLGLKAH

SEQ ID NO: 287 - protein, 8 aa, synthetic construct
GPLGLKAK

SEQ ID NO: 288 - protein, 8 aa, synthetic construct
GPLGLKAS

SEQ ID NO: 289 - protein, 8 aa, synthetic construct
GPLGLFGA

SEQ ID NO: 290 - protein, 8 aa, synthetic construct
GPLGLQGA

SEQ ID NO: 291 - protein, 8 aa, synthetic construct
GPLGLVGA

SEQ ID NO: 292 - protein, 8 aa, synthetic construct
GPLGLAGA

SEQ ID NO: 293 - protein, 8 aa, synthetic construct
GPLGLLGA

SEQ ID NO: 294 - protein, 8 aa, synthetic construct
GPLGLRGA

SEQ ID NO: 295 - protein, 8 aa, synthetic construct
GPLGLYGA

SEQ ID NO: 296 - protein, 8 aa, synthetic construct
ALFKSSPP

SEQ ID NO: 297 - protein, 8 aa, synthetic construct
SPFRSSRQ

SEQ ID NO: 298 - protein, 8 aa, synthetic construct
KLFKSSPP

SEQ ID NO: 299 - protein, 8 aa, synthetic construct
HLFKSSPP

SEQ ID NO: 300 - protein, 8 aa, synthetic construct
SLFKSSPP

SEQ ID NO: 301 - protein, 8 aa, synthetic construct
QLFKSSPP

SEQ ID NO: 302 - protein, 8 aa, synthetic construct
LLFKSSPP

SEQ ID NO: 303 - protein, 8 aa, synthetic construct
PLFKSSPP

SEQ ID NO: 304 - protein, 8 aa, synthetic construct
FLFKSSPP

SEQ ID NO: 305 - protein, 8 aa, synthetic construct
GLFKSSPP

SEQ ID NO: 306 - protein, 8 aa, synthetic construct
VLFKSSPP

SEQ ID NO: 307 - protein, 8 aa, synthetic construct
ELFKSSPP

SEQ ID NO: 308 - protein, 8 aa, synthetic construct
AKFKSSPP

SEQ ID NO: 309 - protein, 8 aa, synthetic construct
AHFKSSPP

SEQ ID NO: 310 - protein, 8 aa, synthetic construct
AGFKSSPP

SEQ ID NO: 311 - protein, 8 aa, synthetic construct
APFKSSPP

SEQ ID NO: 312 - protein, 8 aa, synthetic construct
ANFKSSPP

SEQ ID NO: 313 - protein, 8 aa, synthetic construct
AFFKSSPP

SEQ ID NO: 314 - protein, 8 aa, synthetic construct
AAFKSSPP

SEQ ID NO: 315 - protein, 8 aa, synthetic construct
ASFKSSPP

SEQ ID NO: 316 - protein, 8 aa, synthetic construct
AEFKSSPP

SEQ ID NO: 317 - protein, 8 aa, synthetic construct
ALRKSSPP

SEQ ID NO: 318 - protein, 8 aa, synthetic construct
ALLKSSPP

SEQ ID NO: 319 - protein, 8 aa, synthetic construct
ALAKSSPP

SEQ ID NO: 320 - protein, 8 aa, synthetic construct
ALQKSSPP

SEQ ID NO: 321 - protein, 8 aa, synthetic construct
ALHKSSPP

SEQ ID NO: 322 - protein, 8 aa, synthetic construct
ALPKSSPP

SEQ ID NO: 323 - protein, 8 aa, synthetic construct
ALTKSSPP

SEQ ID NO: 324 - protein, 8 aa, synthetic construct
ALGKSSPP

SEQ ID NO: 325 - protein, 8 aa, synthetic construct
ALDKSSPP

SEQ ID NO: 326 - protein, 8 aa, synthetic construct
ALFFSSPP

SEQ ID NO: 327 - protein, 8 aa, synthetic construct
ALFHSSPP

SEQ ID NO: 328 - protein, 8 aa, synthetic construct
ALFTSSPP

SEQ ID NO: 329 - protein, 8 aa, synthetic construct
ALFASSPP

SEQ ID NO: 330 - protein, 8 aa, synthetic construct
ALFQSSPP

SEQ ID NO: 331 - protein, 8 aa, synthetic construct
ALFLSSPP

SEQ ID NO: 332 - protein, 8 aa, synthetic construct
ALFGSSPP

SEQ ID NO: 333 - protein, 8 aa, synthetic construct
ALFESSPP

SEQ ID NO: 334 - protein, 8 aa, synthetic construct
ALFPSSPP

SEQ ID NO: 335 - protein, 8 aa, synthetic construct
ALFKHSPP

SEQ ID NO: 336 - protein, 8 aa, synthetic construct
ALFKLSPP

SEQ ID NO: 337 - protein, 8 aa, synthetic construct
ALFKKSPP

SEQ ID NO: 338 - protein, 8 aa, synthetic construct
ALFKASPP

SEQ ID NO: 339 - protein, 8 aa, synthetic construct
ALFKISPP

SEQ ID NO: 340 - protein, 8 aa, synthetic construct
ALFKGSPP

SEQ ID NO: 341 - protein, 8 aa, synthetic construct
ALFKNSPP

SEQ ID NO: 342 - protein, 8 aa, synthetic construct
ALFKRSPP

SEQ ID NO: 343 - protein, 8 aa, synthetic construct
ALFKESPP

SEQ ID NO: 344 - protein, 8 aa, synthetic construct
ALFKFSPP

SEQ ID NO: 345 - protein, 8 aa, synthetic construct
ALFKPSPP

SEQ ID NO: 346 - protein, 8 aa, synthetic construct
ALFKSFPP

SEQ ID NO: 347 - protein, 8 aa, synthetic construct
ALFKSLPP

SEQ ID NO: 348 - protein, 8 aa, synthetic construct
ALFKSIPP

SEQ ID NO: 349 - protein, 8 aa, synthetic construct
ALFKSKPP

SEQ ID NO: 350 - protein, 8 aa, synthetic construct
ALFKSAPP

SEQ ID NO: 351 - protein, 8 aa, synthetic construct
ALFKSQPP

SEQ ID NO: 352 - protein, 8 aa, synthetic construct
ALFKSPPP

SEQ ID NO: 353 - protein, 8 aa, synthetic construct
ALFKSEPP

SEQ ID NO: 354 - protein, 8 aa, synthetic construct
ALFKSGPP

SEQ ID NO: 355 - protein, 8 aa, synthetic construct
ALFKSSFP

SEQ ID NO: 356 - protein, 8 aa, synthetic construct
ALFKSSLP

SEQ ID NO: 357 - protein, 8 aa, synthetic construct
ALFKSSGP

SEQ ID NO: 358 - protein, 8 aa, synthetic construct
ALFKSSSP

SEQ ID NO: 359 - protein, 8 aa, synthetic construct
ALFKSSVP

SEQ ID NO: 360 - protein, 8 aa, synthetic construct
ALFKSSHP

SEQ ID NO: 361 - protein, 8 aa, synthetic construct
ALFKSSAP

SEQ ID NO: 362 - protein, 8 aa, synthetic construct
ALFKSSNP

SEQ ID NO: 363 - protein, 8 aa, synthetic construct
ALFKSSKP

SEQ ID NO: 364 - protein, 8 aa, synthetic construct
ALFKSSEP

SEQ ID NO: 365 - protein, 8 aa, synthetic construct
ALFKSSPF

SEQ ID NO: 366 - protein, 8 aa, synthetic construct
ALFKSSPH

SEQ ID NO: 367 - protein, 8 aa, synthetic construct
ALFKSSPG

SEQ ID NO: 368 - protein, 8 aa, synthetic construct
ALFKSSPA

SEQ ID NO: 369 - protein, 8 aa, synthetic construct
ALFKSSPS

SEQ ID NO: 370 - protein, 8 aa, synthetic construct
ALFKSSPV

SEQ ID NO: 371 - protein, 8 aa, synthetic construct
ALFKSSPQ

SEQ ID NO: 372 - protein, 8 aa, synthetic construct
ALFKSSPK

SEQ ID NO: 373 - protein, 8 aa, synthetic construct
ALFKSSPL

SEQ ID NO: 374 - protein, 8 aa, synthetic construct
ALFKSSPD

SEQ ID NO: 375 - protein, 8 aa, synthetic construct
KRALGLPG

SEQ ID NO: 376 - protein, 40 aa, synthetic construct
DEDEDEDEDEDEDEDERPLALWRSDRDRDRDRDRDRDRDR

SEQ ID NO: 377 - skipped

SEQ ID NO: 378 - protein, 5 aa, synthetic construct
LEATA

SEQ ID NO: 379 - protein, 10 aa, synthetic construct
GGAANLVRGG

SEQ ID NO: 380 - protein, 10 aa, synthetic construct
SGRIGFLRTA

SEQ ID NO: 381 - protein, 6 aa, synthetic construct
PLGLAG

SEQ ID NO: 382 - protein, 6 aa, synthetic construct
PLGLAX

SEQ ID NO: 383 - protein, 6 aa, synthetic construct
  MOD_RES 4: note = s-methyl cysteine
PLGCAG

SEQ ID NO: 384 - protein, 8 aa, synthetic construct
ESPAYYTA

SEQ ID NO: 385 - protein, 6 aa, synthetic construct
RLQLKL

SEQ ID NO: 386 - protein, 7 aa, synthetic construct
RLQLKAC

SEQ ID NO: 387 - protein, 7 aa, synthetic construct
  MOD_RES 3: note = citrulline
  MOD_RES 5: note = homophenylalanine
EPXGXYL

SEQ ID NO: 388 - protein, 5 aa, synthetic construct
SGRSA

SEQ ID NO: 389 - protein, 4 aa, synthetic construct
DAFK

SEQ ID NO: 390 - protein, 5 aa, synthetic construct
GGGRR

SEQ ID NO: 391 - protein, 4 aa, synthetic construct
GFLG

SEQ ID NO: 392 - protein, 4 aa, synthetic construct
ALAL

SEQ ID NO: 393-394 - skipped (2 entries)

SEQ ID NO: 395 - protein, 5 aa, synthetic construct
  MOD_RES 3: note = s-ethyl cysteine
PICFF

SEQ ID NO: 396 - protein, 8 aa, synthetic construct
GGPRGLPG

SEQ ID NO: 397 - protein, 6 aa, synthetic construct
HSSKLQ

SEQ ID NO: 398 - protein, 7 aa, synthetic construct
HSSKLQL

SEQ ID NO: 399 - protein, 9 aa, synthetic construct
HSSKLQEDA

SEQ ID NO: 400 - protein, 10 aa, synthetic construct
LVLASSSFGY

SEQ ID NO: 401 - protein, 10 aa, synthetic construct
GVSQNYPIVG

SEQ ID NO: 402 - protein, 10 aa, synthetic construct
GVVQASCRLA

SEQ ID NO: 403 - skipped

SEQ ID NO: 404 - protein, 6 aa, synthetic construct
DPRSFL

SEQ ID NO: 405 - protein, 6 aa, synthetic construct
PPRSFL

SEQ ID NO: 406 - protein, 4 aa, synthetic construct
DEVD

SEQ ID NO: 407 - protein, 5 aa, synthetic construct
DEVDP

SEQ ID NO: 408 - protein, 8 aa, synthetic construct
KGSGDVEG

SEQ ID NO: 409 - protein, 6 aa, synthetic construct
GWEHDG

SEQ ID NO: 410 - protein, 7 aa, synthetic construct
EDDDDKA

SEQ ID NO: 411 - protein, 9 aa, synthetic construct
KQEQNPGST

SEQ ID NO: 412 - protein, 6 aa, synthetic construct
GKAFRR

SEQ ID NO: 413 - protein, 4 aa, synthetic construct
DAFK

SEQ ID NO: 414 - protein, 4 aa, synthetic construct
DVLK

SEQ ID NO: 415 - protein, 4 aa, synthetic construct
DAFK

SEQ ID NO: 416 - protein, 7 aa, synthetic construct
ALLLALL

SEQ ID NO: 417 - protein, 7 aa, synthetic construct
GPLGVRG

SEQ ID NO: 418 - protein, 8 aa, synthetic construct
IPVSLRSG

SEQ ID NO: 419 - protein, 8 aa, synthetic construct
VPLSLYSG

SEQ ID NO: 420 - protein, 10 aa, synthetic construct
SGESPAYYTA

SEQ ID NO: 421 - protein, 609 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 422 - protein, 426 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 423-446 - skipped (24 entries)

SEQ ID NO: 447 - protein, 7 aa, synthetic construct
PAGLYAQ

SEQ ID NO: 448 - protein, 7 aa, synthetic construct
LFKSSFP

(19) United States
(12) Patent Application Publication
(10) Pub. No.: US 2024/0216505 A1
(43) Pub. Date: Jul. 4, 2024

(54) INDUCIBLE CYTOKINE PRODRUG AND PD-1/PD-L1 COMBINATION THERAPY

(71) Applicant: Werewolf Therapeutics, Inc., Watertown, MA (US)

(72) Inventors: William WINSTON, West Newton, MA (US); Daniel HICKLIN, Boston, MA (US); Jose Andres SALMERON-GARCIA, Acton, MA (US); Cynthia SEIDEL-DUGAN, Belmont, MA (US); Heather BRODKIN, West Newton, MA (US); Philipp STEINER, Boston, MA (US)

(21) Appl. No.: 18/440,816

(22) Filed: Feb. 13, 2024

Related U.S. Application Data
(63) Continuation of application No. PCT/US2022/040478, filed on Aug. 16, 2022.
(60) Provisional application No. 63/275,714, filed on Nov. 4, 2021.
(60) Provisional application No. 63/234,001, filed on Aug. 17, 2021.

(51) Int. Cl. A61K 39/395; A61K 38/20; A61K 38/21; A61K 47/64; A61K 47/65; A61K 47/68; A61P 35/00

(52) U.S. Cl. CPC A61K 39/3955; A61K 38/2013; A61K 38/208; A61K 38/212; A61K 38/215; A61K 38/217; A61K 47/643; A61K 47/65; A61K 47/6813; A61K 47/6889; A61P 35/00

(57) ABSTRACT

This disclosure relates to methods and compositions for treating cancer including lymphoma using an inducible cytokine prodrug, and to a combination therapy comprising an inducible cytokine prodrug in combination with anti-PD-1 and anti-PD-L1 antibodies.

### 1. RELATED APPLICATIONS

The present application claims the benefit of U.S. Provisional Application No. 63/234,001, filed on Aug. 17, 2021, and U.S. Provisional Application No. 63/275,714, filed on Nov. 4, 2021, each of which are incorporated by reference in their entirety.

### 2. BACKGROUND

Inducible cytokine prodrugs, that are conditionally activated in the tumor microenvironment through protease cleavage to release the fully active, native cytokine within the tumor to stimulate a potent anti-tumor immune response, are described in International Publication Nos.: WO2019/222294, WO2019/222295, WO2019/222296, WO2021/097376. These prodrugs typically include a native cytokine polypeptide that is attached to a cytokine blocking domain and typically a half-life extension domain, through a protease cleavable linker. For example, IL-2 prodrugs can include a native IL-2 molecule attached through a protease cleavable linker to a half-life extension domain (e.g., anti-human serum albumin antibody binding fragment such as a VH domain) and an IL-2 blocking element (e.g., anti-IL-2 antibody binding fragment, such as a Fab) to block binding of IL-2 to IL-2β/γ receptors on normal tissue in the periphery. Upon cleavage of the protease cleavable linker in the tumor microenvironment, fully active native IL-2 is released within the tumor to stimulate a potent anti-tumor immune response.

Immune checkpoint inhibitors are proteins that regulate T cell functions. T cell effector function is important for immunotherapeutic approaches to treating tumors. But immunosuppression, and decreased effector function, is often seen as tumors grow and cancer progresses. One mechanism behind this phenomenon is the activation of immune checkpoint inhibitors by cancer cells, leading to suppression of the anti-tumor immune response. This typically occurs when cancer cells express proteins on their surface that can interact with immune checkpoint proteins on the surface of T cells in the tumor microenvironment to suppress the activity of the T cells. Immune checkpoint proteins include, for example, PD-1 which binds ligands PD-L1 (B7-H1, CD274) and PD-L2 (B7-DC, CD273), CTLA-4 (CD152) which binds B7-1 (CD80) and B7-2 (CD86), LAG 3 (CD223) which binds Galectin3, LSECtin and FGL1; TIM3 (HAVCR2) which binds ligands CeacamI and Galectin9; TIGIT (VSTM3, WUCAM) which binds CD112 and CD155; BTLA (CD272) which binds HVEM (TNFRSF14), B7-H3 (CD276), B7-H4 (VTCN1), VISTA (B7-H5), KIR, CD44 (2B4), CD160 (BY55) which bind HVEM; CD134 (TNRFSR4, OX40) which binds CD252 (OX-40L).

Therapeutic agents, such as antibodies, that bind immune checkpoint proteins and inhibit their immunosuppressive activity have been developed as anti-tumor agents. Several such agents are now commercially available for cancer therapy, including the anti-PD1 antibodies pembrolizumab (KEYTRUDA), dostarlimab (JEMPERLI), cemiplimab-rwlc (LIBATYO), nivolumab (OPDIVO), camrelizumab, tislelizumab, toripalimab, and sintilimab (TYVYT); the anti-PD-L1 antibodies avelumab (BAVENCIO), durvalumab (IMFINZI), and atezolizumab (TECENTRIQ); the anti-CTLA-4 antibody ipilimumab (YERVOY). While therapy with such immune checkpoint inhibitors provide advantages for cancer therapy, the overall success remains low, relapse occurs and resistance to checkpoint inhibition develops.

Lymphomas are a heterogenous group of hematological malignancies originating from lymphoid cells. In lymphoma the normal balance between activation and suppression of the immune system is frequently shifted by the tumor cells toward profound immune suppression, which can protect tumor cells from T-cell mediated killing. (Ansell, SM Hematology Am Soc, Hematol Educ Program (2017), 2017(1):618-621.) Immune modulation using agonist antibodies (e.g., anti-CD27, anti-CD40, anti-CD137) has been used in attempts to treat lymphomas, but with only modest therapeutic benefit seen in clinical studies, highlighting the challenge of overcoming the immunosuppressive tumor microenvironment in lymphoma. (Id.)

There is an unmet medical need for improved methods for treating cancer.

### 3. SUMMARY

This disclosure relates to methods of treating lymphoma in a subject comprising administering an effective amount of an inducible cytokine prodrug optionally in combination with an additional therapeutic, such as a chemotherapeutic agent and/or an immune checkpoint inhibitor (e.g. an anti-PD-1 antibody or an anti-PD-L1 antibody). The disclosure also relates to a combination comprising an inducible cytokine prodrug and an additional therapeutic, such as a chemotherapeutic agent and/or an immune checkpoint inhibitor (e.g. an anti-PD-1 antibody or an anti-PD-L1 antibody) that can be used for treating lymphoma.

This disclosure relates to methods of treating cancer in a subject comprising administering an effective amount of an inducible cytokine prodrug and an anti-PD-1 antibody or an anti-PD-L1 antibody. The disclosure also relates to a combination comprising an inducible cytokine prodrug and an anti-PD-1 antibody or an anti-PD-L1 antibody that can be used for treating cancer. In some instances, an anti-PD-1 antibody will be combined with the inducible cytokine prodrug. In some instances, an anti-PD-L1 antibody will be combined with the inducible cytokine prodrug.

The inducible cytokine prodrug comprises cytokine polypeptide [A], a blocking element [D], optionally a half-life extension element [H] and a protease-cleavable polypeptide linker. The cytokine polypeptide and the cytokine blocking element and the optional half-life extension element when present are operably linked by the protease-cleavable polypeptide linker and the inducible cytokine prodrug has attenuated cytokine receptor activating activity. The cytokine-receptor activating activity of the inducible cytokine prodrug is at least about 10× less than the cytokine receptor activating activity of the polypeptide that contains the cytokine polypeptide that is produced by cleavage of the protease cleavable linker.

The cytokine polypeptide can be IL-2, IL-12, interferon alpha, interferon beta, interferon gamma, or a mutein, or an active fragment of any of the foregoing. The cytokine polypeptide can be IL-2, or a mutein, a variant, an active fragment, or a subunit of any of the foregoing. The cytokine polypeptide can be IL-12, or a mutein, a variant, an active fragment, or a subunit of any of the foregoing. The cytokine polypeptide can be interferon alpha, interferon beta, interferon gamma, or a mutein, or an active fragment of any of the foregoing.

The inducible cytokine prodrug can have the formula:

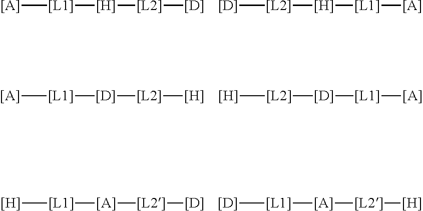

A is a cytokine polypeptide, D is a blocking moiety, H is a half-life extension moiety, L1 is a protease-cleavable polypeptide linker, L2 is an polypeptide linker that is optionally protease-cleavable, and L2′ is a protease-cleavable polypeptide linker.

The inducible cytokine prodrug can be a single polypeptide chain. The inducible cytokine prodrug can comprise at least two polypeptide chains. The inducible cytokine prodrug can comprise at least three polypeptide chains.

The cytokine prodrug can comprise Compound 1, Compound 2, Compound 3, Compound 4, or an amino acid sequence variant of the foregoing. Compound 1 can comprise a first polypeptide chain of SEQ ID NO:1 and a second polypeptide chain of SEQ ID NO:5, and the amino acid sequence variant of Compound 1 can comprise a first polypeptide chain that has at least about 80% identity to SEQ ID NO:1 and a second polypeptide chain that has at least about 80% identity to SEQ ID NO:5. Compound 2 can comprise a first polypeptide chain of SEQ ID NO:2 and a second polypeptide chain of SEQ ID NO:5, and the amino acid sequence variant of Compound 2 can comprise a first polypeptide chain that has at least about 80% identity to SEQ ID NO:2 and a second polypeptide chain that has at least about 80% identity to SEQ ID NO:5. Compound 3 can comprise a first polypeptide chain of SEQ ID NO:3 and a second polypeptide chain of SEQ ID NO:5, and the amino acid sequence variant of Compound 3 can comprise a first polypeptide chain that has at least about 80% identity to SEQ ID NO:3 and a second polypeptide chain that has at least about 80% identity to SEQ ID NO:5. Compound 4 can comprise a first polypeptide chain of SEQ ID NO:1 and a second polypeptide chain of SEQ ID NO:4, and the amino acid sequence variant of Compound 4 can comprise a first polypeptide chain that has at least about 80% identity to SEQ ID NO:4 and a second polypeptide chain that has at least about 80% identity to SEQ ID NO:5.

The inducible cytokine prodrug can be Compound 5, Compound 6, Compound 7, Compound 8, Compound 9, or Compound 10, or an amino acid sequence variant of the foregoing. Compound 5 can comprise a first polypeptide chain of SEQ ID NO: 6 and a second polypeptide chain of SEQ ID NO:12, and the amino acid sequence variant of Compound 5 can comprise a first polypeptide chain that has at least about 80% identity to SEQ ID NO: 6 and a second polypeptide chain that has at least about 80% identity to SEQ ID NO: 12. Compound 6 can comprise a first polypeptide chain of SEQ ID NO: 7 and a second polypeptide chain of SEQ ID NO: 12, and the amino acid sequence variant of Compound 6 can comprise a first polypeptide chain that has at least about 80% identity to SEQ ID NO:7 and a second polypeptide chain that has at least about 80% identity to SEQ ID NO: 12. Compound 7 can comprise a first polypeptide chain of SEQ ID NO: 8 and a second polypeptide chain of SEQ ID NO: 13, and the amino acid sequence variant of Compound 7 can comprise a first polypeptide chain that has at least about 80% identity to SEQ ID NO: 8 and a second polypeptide chain that has at least about 80% identity to SEQ ID NO: 13. Compound 8 can comprise a first polypeptide chain of SEQ ID NO: 9 and a second polypeptide chain of SEQ ID NO: 13, and the amino acid sequence variant of Compound 8 can comprise a first polypeptide chain that has at least about 80% identity to SEQ ID NO: 9 and a second polypeptide chain that has at least about 80% identity to SEQ ID NO: 13. Compound 9 can comprise a first polypeptide chain of SEQ ID NO: 10 and a second polypeptide chain of SEQ ID NO: 13, and the amino acid sequence variant of Compound 9 comprises a first polypeptide chain that has at least about 80% identity to SEQ ID NO: 10 and a second polypeptide chain that has at least about 80% identity to SEQ ID NO: 13. Compound 10 can comprise a first polypeptide chain of SEQ ID NO: 11 and a second polypeptide chain of SEQ ID NO: 13, and the amino acid sequence variant of Compound 10 can comprise a first polypeptide chain that has at least about 80% identity to SEQ ID NO: 11 and a second polypeptide chain that has at least about 80% identity to SEQ ID NO: 1.

The inducible cytokine prodrug can comprise the amino acid selected from 14-20, or an amino acid sequence that has at least about 80% identity to SEQ ID NOs 14-20.

The half-life extension element can be a human serum albumin, an antigen binding polypeptide that binds human serum albumin, or an immunoglobulin Fc or fragment thereof.

The protease cleavable linker can comprise a sequence that is capable of being cleaved by a protease selected from kallikrein, thrombin, chymase, carboxypeptidase A, cathepsin, elastase, PR-3, granzyme M, a calpain, a matrix metalloproteinase (MMP), an ADAM, a FAP, a plasminogen activator, a caspase, a tryptase, or a tumor protease.

In particular, the protease can be selected from cathepsin B, cathepsin C, cathepsin D, cathepsin E, cathepsin K, cathepsin L, or cathepsin G. In particular, the protease can be selected from matrix metalloprotease (MMP) is MMP1, MMP2, MMP3, MMP8, MMP9, MMP10, MMP11, MMP12, MMP13, or MMP14.

The blocking element can bind the cytokine polypeptide. The blocking element can comprise a ligand-binding domain or fragment of a cognate receptor for the cytokine polypeptide, an antibody or antigen-binding fragment of an antibody that binds to the cytokine polypeptide. The antibody or antigen-binding fragment can be a single domain antibody, a Fab, or a scFv that binds the cytokine polypeptide.

The inducible cytokine prodrug can be administered before, concurrently with or after the additional therapeutic agent, e.g. chemotherapeutic agent, immune checkpoint inhibitor (such as anti-PD-1 antibody or anti-PD-L1 antibody).

The anti-PD-1 antibody can be selected from the group consisting of AMP-224 (AstraZeneca), 609A (3SBio), 704 (3SBio), 705 (3SBio), ABBV-181 (AbbVie), ADU-1503/bion-004 (Chinook Therapeutics), AGEN2034/balstilimab (Agenus), AK103 (Akeso), AK104 (Akeso), AK 112 (Akeso), AK123 (Akeso), AMG 256 (Amgen), AMG 404 (Amgen), ANB030 (AnaptysBio), ANKEBIO Anti-PD1 product (Anhui Anke Biotechnology), Anti PD-1/Anti-CD47 (DiNonA), ASKG915 (Ask Gene Pharmaceuticals), AV-MEL-1 (Aivita Biomedical), BCD-100 (Biocad CJSC), BI 754091 (Boehringer Ingelheim), BiCKI-IL-7 (OSE Immunotherapeutics), Boehringer-PD-1-unknown (Boehringer Ingelheim), BSK-050K01 (Biosion), Camrelizumab (Jiangsu Hengrui Medicine), CB201 (Crescendo Biologics), CB213 (Crescendo Biologics), CC-90006 (AnaptsBio), cetrelimab (J&J), chPD1 (Kiromic Biopharma), CMAB819 (Mabpharm), CS1003 (CStone Pharmaceuticals), CS17938 (Shenzhen Chipscreen Biosciences), CTX-8371 (Compass Therapeutics), CX-072 (CytomX Therapeutics), CX-188 (CytomX Therapeutics), cypalizumab (Harbin Gloria Pharmaceuticals), DB004 (DotBio), EMB02 (EpimAb Biotherapeutics), Geptanblimab/genolimzumab (Apollomics), GS19 (Suzhou Zelgen Biopharmaceuticals), HLX10 (Shanghai Henlius Biotech), HX008 (Taizhou HanZhong Pharmaceuticals), HY003 (Juventas Cell Therapy), IBI315/BH2950 (Innovent Biologics), IBI318 (Innovent Biologics), IBI319 (Innovent Biologics), IMM1802 (ImmuneOnco Biopharma), IMT200 (TrueBinding), Jemperli/dostarlimab (AnaptysBio), JTX-4014 (Jounce Therapeutics), Keytruda/pembrolizumab (Merck), LBL-006 (Nanjing Leads Biolabs), Libtayo/cemiplimab-rwlc (Regeneron Pharmaceuticals), LVGN3616 (Lyvgen Biopharma), LXF821 (Novartis), LY01015 (Luye Pharma Group), LY3462817 (Eli Lilly), MCLA-134 (Merus N.V.), MEDI5752 (AstraZenica), NIR178 (Novartis), ONCR-177 (Oncorus), ONO-4685 (Ono Pharmaceutical), Opdivo/nivolumab (Ono Pharmaceutical), MGD019 (MacroGenius), PD1-GDT CAR-T (Kiromic Biopharma), penpulimab (Akeso), PSB205 (Qilu Puget Sound Biotherapeutics), PT-001 (Merck), PT627 (Merck), RB-M1 (Refuge Biotechnologies), Retifanlimab (MacroGenics), RG6139 (Roche), RG6279 (Roche), RTX-002 (RubrYc Therapeutics), sasanlimab (Pfizer), Servier-PD1×LAG3-unknown (Servier), SL-279252/TAK-252 (Shattuck Labs), Sofusa anti-PD1 (Sorrento Therapeutics), spartalizumab (Novartis), SSI-361 (Lyvgen Biopharma), Sym021 (Servier), Tebotelimab (MacroGenics), tislelizumab (BeiGene), TSR-075 (AnaptsBio), Tuhura-DO/PD-1-unknown (Tuhura Biopharma), toripalimab (Shanghai Junshi Biosciences), sintilimab (Innovent Biologics), Unicar-CAR-T&PD-1-unknown (Shanghai Unicar-Therapy Bio-Medicine Technology), Xdivane (Xbrane Biopharma), XmAb20717 (Xencor), XmAb23104 (Xencor), YBL-006 (Y-Biologics), zimberelimab (Arcus Biosciences).

Pembrolizumab, dostarlimab, cemiplimab-rwlc, nivolumab, camrelizumab, tislelizumab, toripalimab, sintilimab or a biosimilar of any of the foregoing are preferred anti-PD-L1 antibodies.

The anti-PD-L1 antibody can be chosen from the group consisting of A167 (Sichuan Kelun), ABL501 (ABL Bio), ABL503 (ABL Bio), ABSK041 (Abbisko Therapeutics), ACE1708 (Acepodia), ACE-NK-PDL1 (Acepodia), ADG104 (Adagene), AK106 (Akeso), ALPN-202 (Alpine Immune Sciences), AN4005 (Adlai Nortye Biopharma), BMS-936559/MDX-1105 (BMS), APL-502/TQB2450 (Apollomics), Arbutus-PD-L1-unknown (Arbutus Biopharma), ASC22 (Ascletis Pharma), ATG-101 (Antengene), AVA-004 (Avacta Group), AVA021 (Avacta Group), AVA027 (Avacta Group), AVA-040-100 (Avacta Group), AVA04-Vbp (Avacta Group), Bavencio/avelumab (Merck), BCD-135 (Biocad CJSC), BGB-A333 (BeiGene), Bintrafusp alfa/GSK4045154 (Merck), CA-170/aupm-170 (Dr. Reddy's Laboratories), CCX559 (ChemoCentryx), CDR101 (CDR-Life), cosibelimab (Checkpoint Therapeutics), CTX-8371 (Compass Therapeutics), DiNonA-Solid Tumors-unknown (DiNonA), DR30207 (Zhejiang Doer Biologics), DuoBody-PD-L1×4-1BB (Ligand Pharmaceuticals), envafolimab (Alphamab Oncology), EPIM-001 (Elpis Biopharmaceuticals), ES101 (Elpiscience Biopharma), INBRX-105 (Inhibrx), FAZ053 (Novartis), FS118 (F-star Therapeutics), GB262 (Genor Biopharma), GS-4224 (Gilead), GT900008 (Kintor Pharmaceuticals), GX-P2 (Genexine), Hamni-PS-L1/CD47-Unknown (Hanmi Pharmaceutical), HBM7015 (HBM Holdings), HBM9167 (HBM Holdings), HLX20 (Shanghai Henlius Biotech), HTI-1088 (Jiangsu Hengrui Medicine), IBI318 (Innovent Biologics), IBI322 (Innovent Biologics), IBI323 (Innovent Biologics), IGM-7354 (IGM Biosciences), IMC-001 (Sorrento Therapeutics), Imfinzi/durvalumab (AstraZenica), IMM25 (ImmuneOnco Biopharma), IMM2502 (ImmuneOnco Biopharma), IMM2503 (ImmuneOnco Biopharma), IMM2504 (ImmuneOnco Biopharma), INCB86550 (Incyte), I0103 (IO Biotech), JS003 (Shanghai Junshi Biosciences), Jubilant-PD-L1-unknown (Jubilant Therapeutics), KD033 (Kadmon Holdings), KN046 (Alphamab Oncology), KY1003 (Sanofi), KY1043 (Sanofi), LY3300054 (Eli Lilly), LY3415244 (Eli Lilly), MRNA-6981 (Moderna), MSB2311 (Transcenta Holding), MT-6035 (Molecular Templates), ND021/NM21-1480 (Numab Therapeutics), OXOOIR (Oxford BioTherapeutics), PD-L1 based BsAbs (I-Mab), PD-L1 Boltbody ISAC (Bolt Biotherapeutics), PDL-GEX (Glycotope GmbH), PMC-122 (PharmAbcine), PMI06 (D&D Pharmatech), Protheragen-RV-scFv-PDL1-unknown (Protheragen), PRS-344 (Pieris Pharmaceuticals), Q-1802 (Merck), RC98 (Yantai Rongchang Pharmaceutical), RV-scFv-PDL1 (Protheragen), SenI_TAAx22P (Hebei Senlang Biotechnology), SHC020 (Nanjing Sanhome Pharmaceutical), sugemalimab (Ligand Pharmaceuticals), atezolizumab (Roche), TST005 (Transcenta Holding), TT-O1 (Topmunnity Therapeutics), TTX-siPDL1 (TransCode Therapeutics), UniCAR-T-PD-L1 (GEMoaB monoclonals), Vaximm (VXM10), and YBL-013 (Y-Biologics).

Avelumab, durvalumab, atezolizumab or a biosimilar of any of the foregoing are preferred anti-PD-1 antibodies.

The chemotherapeutic agent can be cyclophosphamide, mechlorethamine, melphalan, chlorambucil, ifosfamide, busulfan, N-Nitroso-N-methylurea (MNU), carmustine (BCNU), lomustine (CCNU), semustine (MeCCNU), fotemustine, streptozotocin, dacarbazine, mitozolomide, temozolomide, thiotepa, mitomycin, diaziquone (AZQ), cisplatin, carboplatin, oxaliplatin, procarbazine, hexamethylmelamine, methotrexate, pemetrexed, fluorouracil (e.g. 5-fluorouracil), capecitabine, cytarabine, gemcitabine, decitabine, azacitidine, fludarabine, nelarabine, cladribine, clofarabine, pentostatin, thioguanine, mercaptopurine, vincristine, vinblastine, vinorelbine, vindesine, vinflunine, paclitaxel, docetaxel, etoposide, teniposide, doxorubicin, daunorubicin, epirubicin, idarubicin, pirarubicin, aclarubicin, mitoxantrone, actinomycin, bleomycin, bisantrene, gemcitabine and/or cytarabine, and any combinations thereof, for example.

The methods disclosed herein can be suitable for any cancer. Exemplary cancers include, but are not limited to, adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor. The methods are preferably useful for colon cancer, lung cancer, melanoma, renal cell carcinoma, or breast cancer. The cancer can be melanoma, non-small cell lung cancer (NSCLC), small cell lung cancer (SCLC), head and neck squamous cell cancer (HNSCC), classical Hodgkin lymphoma (cHL), primary mediastinal large B cell lymphoma (PMBCL), microsatellite instability high or mismatch repair deficient cancer, microsatellite instability high or mismatch repair deficient colorectal cancer, gastric cancer, esophageal cancer, cervical cancer, hepatocellular carcinoma (HCC), merkel cell carcinoma (MCC), renal cell carcinoma (RCC), endometrial carcinoma, tumor mutational burden high cancer, cutaneous squamous cell carcinoma (cSCC), triple negative breast cancer (TNBC), urothelial carcinoma, colorectal cancer or oesophageal carcinoma. The cancer can be metastatic renal clear cell carcinoma or metastatic cutaneous malignant melanoma.

In certain embodiments, the cancer is a lymphoma, such as a B cell lymphoma, a T cell lymphoma, Non-Hodgkin Lymphoma, Hodgkin Lymphoma, diffuse large B-cell lymphoma, primary mediastinal B cell lymphoma, follicular lymphoma, small lymphocytic lymphoma, chronic lymphocytic leukemia, marginal zone lymphoma, mantle cell lymphoma, Waldenstrom's macroglobulinemia, Burkitt lymphoma, peripheral T cell lymphoma, anaplastic large cell lymphoma, angioimmunoblastic lymphoma, cutaneous T cell lymphoma, central nervous system lymphoma, grey zone lymphoma, double hit lymphoma, triple hit lymphoma, high grade B cell lymphomas not otherwise specified, lymphoblastic lymphoma, lymphoplasmacytic lymphoma, MALT lymphoma, monocytoid B cell lymphoma, natural killer (NK) cell lymphoma, mycosis fungoides, Sezary syndrome, enteropathy-type T cell lymphoma, hepatosplenic gamma/delta T cell lymphoma, and the like.

In particular embodiments, the cancer is selected from the group consisting of: melanoma, non-small cell lung cancer, head and neck squamous cell cancer, classical Hodgkin lymphoma, primary mediastinal large B-cell lymphoma, urothelial carcinoma, microsatellite instability-high or mismatch repair deficient cancer, gastric cancer, esophageal cancer, cervical cancer, hepatocellular carcinoma, Merkel cell carcinoma, renal cell carcinoma, endometrial carcinoma, a cancer characterized by a tumor having a high mutational burden, cutaneous squamous cell carcinoma, and triple negative breast cancer.

The disclosure also relates to pharmaceutical compositions comprising the inducible cytokine prodrug an additional therapeutic, such as a chemotherapeutic agent and/or an immune checkpoint inhibitor (e.g. an anti-PD-1 antibody or an anti-PD-L1 antibody) and a suitable carrier. The disclosure also relates to pharmaceutical compositions comprising the inducible cytokine prodrug, an anti-PD-1 or an anti-PD-L1 antibody, and a suitable carrier. The pharmaceutical composition can be a liquid composition for intravenous administration. The pharmaceutical composition can be a lyophilized composition. The pharmaceutical composition can be a lyophilized composition is for reconstitution using water for formulation is suitable of intravenous administration.

### 4. BRIEF DESCRIPTION OF DRAWINGS

FIGS. 1A-1F shows data (tumor volume and/or body weight) for mice treated with inducible IL-2 prodrugs, anti-PD1 antibody, or inducible IL-2 prodrugs and anti-PD1 antibody.

FIG. 2A-2B. shows data (tumor volume and/or body weight) for mice treated with inducible IL-2 prodrugs, anti-PD1 antibody, or inducible IL-2 prodrugs and anti-PD1 antibody.

FIG. 3A is a graph showing tumor volume progression in a B16F10 tumor model in which mice were treated with 100 μg of compound 1, 200 μg of compound 1, or only vehicle. 1×10⁵ tumor cells were implanted in the flank of animals and tumor growth was monitored. Once tumors reached an average volume of 30-60 mm³, the animals were randomized and dosed. Tumor volumes and body weights were recorded three times per week.

FIG. 3B is a graph showing tumor volume progression in a B16F10 tumor model in which mice were treated with 100 μg of compound 1, 200 μg of anti-PD-1 antibody (RMP1-14) alone, 100 μg of compound 1 and 200 μg of anti-PD-1 antibody (RMP1-14), or only vehicle. 1×10⁵ tumor cells were implanted in the flank of animals and tumor growth was monitored. Once tumors reached an average volume of 30-60 mm³, the animals were randomized and dosed. Tumor volumes and body weights were recorded three times per week.

FIG. 4 is a graph showing body weight progression in a B16F10 tumor model in which mice were treated with compound 1, compound 1 and RMP1-14, or only vehicle. 1×105 tumor cells were implanted in the flank of animals and tumor growth was monitored. Once tumors reached an average volume of 30-60 mm³, the animals were randomized and dosed. Body weights were recorded three times per week.

FIG. 5A is a graph showing results of analyzing inducible IL-12 prodrug (WW0757/636) in a syngeneic A20 mouse tumor model. The graph shows average tumor volume over time in mice treated with 30 g WW0757/636 (diamond), 100 g WW0757/636 (square) and 300 g WW0757/636 (circle). Vehicle alone is indicated by a black circle (top line with largest tumor volume). The data show tumor volume increase was inhibited over time in a dose-dependent manner in mice treated with WW0757/636 at the higher concentrations.

FIG. 5B is a graph showing results of analyzing inducible IFNα prodrug WW0610 in a syngeneic A20 mouse tumor model. It shows average tumor volume over time in mice treated with 45 g WW0610 (diamond), and 133 g WW0610 (square). Vehicle alone is indicated by a black circle (top line with largest tumor volume). The data show tumor volume increase was inhibited over time in a dose-dependent manner in mice treated with WW0610 at the higher concentrations.

FIG. 6A is a graph showing results of analyzing inducible IL-12 prodrug (WW0757/636) in a syngeneic EG7.OVA mouse tumor model. It shows average tumor volume over time in mice treated with 30 g WW0757/636 (diamond), 100 g WW0757/636 (square) and 300 g WW0757/636 (circle). Vehicle alone is indicated by a black circle (top line with largest tumor volume data through day 18). The data show tumor volume increase was inhibited over time in a dose-dependent manner in mice treated with WW0757/636 at the higher concentrations.

FIG. 6B is a graph showing results of analyzing inducible IFNα prodrug (WW0610) in a syngeneic EG7.OVA mouse tumor model. It shows average tumor volume over time in mice treated with 45 g WW0610 (diamond), and 133 g WW0610 (square). Vehicle alone is indicated by a black circle (top line with largest tumor volume data through day 18). The data show tumor volume increase was inhibited over time in a dose-dependent manner in mice treated with WW0610 at the higher concentrations.

FIG. 7A is a graph showing average tumor volumes in mice treated with vehicle (PBS, circles), inducible IL-2 prodrug (WW0621/WW0523, squares), anti-PD-1 (triangles), or WW0621/WW0523 and anti-PD-1 (diamonds). The dosing schedule is also shown (arrows).

FIGS. 7B-7E are graphs of tumor volume in individual mice treated with vehicle (FIG. 7B), WW0621/WW0523 (FIG. 7C), anti-PD-1 (FIG. 7D), or WW0621/WW0523 and anti-PD-1 (FIG. 7E).

FIG. 7F is a graph showing average body weights of mice treated with vehicle (PBS circles), inducible IL-2 prodrug (WW0621/WW0523, squares), anti-PD-1 (triangles), and WW0621/WW0523 and anti-PD-1 (diamonds).

### 5. DETAILED DESCRIPTION

### A. Inducible Cytokines

The disclosure relates to inducible cytokine prodrugs that contain at least one polypeptide chain, and can contain two or more polypeptides, if desired. The inducible cytokine prodrug comprises a cytokine (e.g., IL-2, IL-12, or IFN), a blocking element, a protease cleavable linker, and a half-life extension element. Any cytokine of interest can be suitable for the inducible cytokine polypeptide prodrugs of this disclosure. Exemplary cytokines include, but are not limited to, interleukins such as IL-2, IL-7, IL-12, IL-15, IL-18, IL-21 IL-23, IFN-alpha (e.g., human IFN-alpha1, human IFN-alpha2, human IFN-alpha4, human IFN-alpha5, human IFN-alpha6, human IFN-alpha7, human IFN-alpha8, human IFN-alpha10, human IFN-alpha13, human IFN-alpha14, human IFN-alpha16, human IFN-alpha17, human IFN-alpha2), IFN-beta, IFN-kappa, or IFN-epsilon, lymphotoxin, TGF-beta1, TGFbeta2, TGFbeta3, GM-CSF, CXCL10, CCL19, CCL20, CCL21 and functional fragments or muteins of any of the foregoing. Preferred cytokines for use in the inducible cytokine prodrugs disclosed herein are IL-2, IL-12, IFN, muteins, functional variants, and functional fragments, or subunits of any of the foregoing.

Inducible cytokine (e.g., IL-2, IL-12, or IFN) prodrugs of this disclosure have attenuated cytokine receptor agonist activity and the circulating half-life is extended. The inducible cytokine receptor agonist activity is attenuated through the blocking element. The half-life extension element can also contribute to attenuation, for example through steric effects. The blocking element is capable of blocking all or some of the receptor agonist activity of the cytokine by noncovalently binding to the cytokine (e.g., to IL-2, IL-12, or IFN) and/or sterically blocking receptor binding. Upon cleavage of the protease cleavable linker a form of the cytokine is released that is active (e.g., more active than the cytokine polypeptide prodrug). Typically, the released cytokine is at least 10× more active than the cytokine polypeptide prodrug. Preferably, the released cytokine is at least 20×, at least 30×, at least 50×, at least 100×, at least 200×, at least 300×, at least 500×, at least 1000×, at least about 10,000× or more active than the cytokine polypeptide complex.

The form of cytokine that is released upon cleavage of the inducible cytokine prodrug typically has a short half-life, which is often substantially similar to the half-life of naturally occurring cytokine. Even though the half-life of the inducible cytokine prodrug is extended, toxicity is reduced or eliminated because the agonist activity of the circulating inducible cytokine prodrug is attenuated and active cytokine is targeted to the desired site of activity (e.g., tumor microenvironment).

It will be appreciated by those skilled in the art, that the number of polypeptide chains, and the location of the elements, the half-life extension element, the protease cleavable linker(s), and the blocking element (and components of such elements, such as a VH or VL domain) on the polypeptide chains can vary and is often a matter of design preference. All such variations are encompassed by this disclosure.

The inducible cytokine prodrug can comprise a single polypeptide chain. Typically, the single polypeptide complex comprises a cytokine polypeptide [A], a blocking element [D], a half-life extension element [H], and a protease cleavable linker [L]. The cytokine [A] polypeptide can be operably linked to the blocking element, the half-life extension element or both the blocking element and the half-life extension element by a protease cleavable linker.

For example, the polypeptide can be of any of Formulas (I)-(VI).

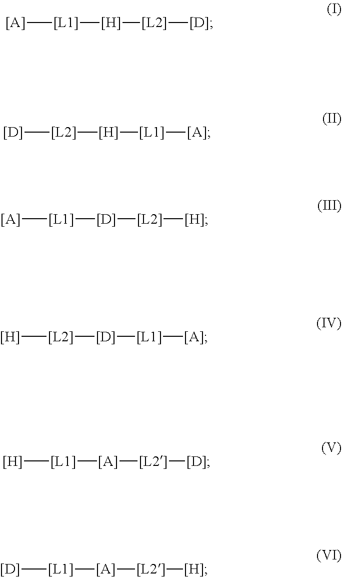

In Formulas (I)-(VI), [A] is a cytokine polypeptide, [D] is a blocking element, [H] is a half-life extension element, [L1] is a protease-cleavable polypeptide linker, [L2] is an polypeptide linker that is optionally protease-cleavable, and [L2′] is a protease-cleavable polypeptide linker. [L1] and [L2] or [L1] and [L2′] can be have the same or different amino acid sequence and or protease-cleavage site (when L2 is protease-cleavable) as desired.

SEQ ID NOs: 21-30 are specific examples of inducible IL-2 prodrugs encompassed by Formulas (I)-(VI) and for use according to this disclosure. SEQ ID NOs. 21-30 and additional details regarding their activity is disclosed in International Publication No.: WO2021/097376.

SEQ ID NOs: 31-40 are specific examples of inducible IL-12 prodrugs encompassed by Formulas (I)-(VI) and for use according to this disclosure. SEQ ID NOs.: 31-40 and additional details regarding their activity is disclosed in International Application No.: PCT/US2021/33014 and International Publication No.: WO2019/222295.

In some instances, the single polypeptide complex comprises a cytokine polypeptide [A], a blocking element (i.e., a steric blocking polypeptide) [D], a protease cleavable linker [L], and an optional half-life extension element. The blocking element [D] can be, for example, HSA or an antibody or antibody fragment (e.g. scFv) that binds HSA.

As an example, the polypeptide can be of Formula (VII): [D]-[L1]-[A]-[L2]-[D]. SEQ ID NOs: 14-20 are specific examples, of inducible IFN prodrugs for use according to this disclosure. SEQ ID NOs: 14-20 and additional details regarding their activity is disclosed in International Application No.: PCT/US2020/060624.

In some instances, the inducible IFN prodrug can comprise IFN polypeptide [A], a blocking element [D], a half-life extension element [H], and a protease cleavable linker [L]. The IFN [A] polypeptide can be operably linked to the blocking element, the half-life extension element or both the blocking element, the half-life extension element by a protease cleavable linker.

IFN polypeptide and the blocking element and the half-life extension element are operably linked by the protease-cleavable polypeptide. For example, the polypeptide can be of any of Formulas (I)-(IX).

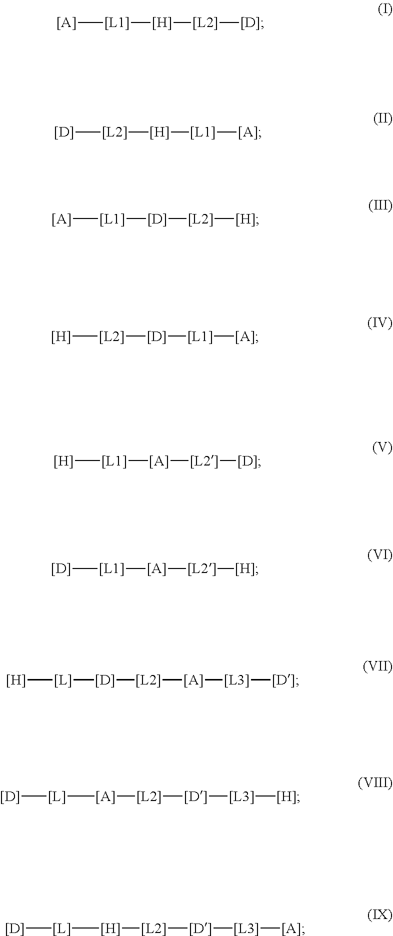

In Formulas (I)-(IX), [A] is a IFN polypeptide, [D] is a IFN blocking element (e.g., extracellular portion of the INFalpha receptor 1 (IFNAR1) or IFNalpha receptor 2 (IFNAR2)), [D′] is either the INFalpha receptor 1 (IFNAR1) or the IFNalpha receptor 2 (IFNAR2) that is not present in [D], [H] is a half-life extension element, [L1] is a protease-cleavable polypeptide linker, [L2] is an polypeptide linker that is optionally protease-cleavable, and [L2′] is a protease-cleavable polypeptide linker. [L1] and [L2] or [L1] and [L2′] can be have the same or different amino acid sequence and or protease-cleavage site (when L2 is protease-cleavable) as desired.

While the inducible cytokine prodrugs disclosed herein preferably contain one half-life extension element and one blocking element, such elements can contain two or more components that are present on the same polypeptide chain or on different polypeptide chains. Illustrative of this, and as disclosed and exemplified herein, components of the blocking element can present on separate polypeptide chains. For example, a first polypeptide chain can include an antibody light chain (VL+CL) or light chain variable domain (VL) and a second polypeptide can include an antibody heavy chain Fab fragment (VH+CH1) or heavy chain variable domain (VH) that is complementary to the VL+CL or VL on the first polypeptide. In such situations, these components can associate in the peptide complex to form an antigen-binding site, such as a Fab that binds to the cytokine (e.g., IL-2, IL-12, or IFN) and attenuates the cytokine activity.

For example, the inducible cytokine prodrug can have a first polypeptide of Formulas (X-XI). Formula X: [D]-[L]-[A]-[L2]-[H] or Formula XI: [H]-[L]-[A]-[L2]-[D]. In Formulas (X)-(XI), [A] is a cytokine polypeptide, [D] is an antibody heavy chain Fab fragment (VH+CH1) or heavy chain variable domain (VH), [H] is a half-life extension element, [L1] is a protease-cleavable polypeptide linker, [L2] is an polypeptide linker that is optionally protease-cleavable, and [L2′] is a protease-cleavable polypeptide linker. [L1] and [L2] or [L1] and [L2′] can be have the same or different amino acid sequence and or protease-cleavage site (when L2 is protease-cleavable) as desired. A second polypeptide antibody light chain (VL+CL) or light chain variable domain (VL) that is complementary to the VH+CH1 or VH.

For instances, an inducible cytokine prodrug can comprise two polypeptide chains. The first polypeptide chain can comprise from the cytokine polypeptide, a protease cleavable linker, half-life extension element (e.g., an anti-human serum albumin (HSA) binding single antibody variable domain), and a VH and CH1 of an antibody that binds the cytokine (e.g., IL-2, a IL-12 subunit i.e., p35, p40, or the p35p40 heterodimeric complex, or IFN). The second polypeptide chain can comprise a VL and CL of an antibody that binds the cytokine (e.g., IL-2, IL-12 subunit (i.e., p35, p40, or the p35p40 heterodimeric complex), or IFN) and that together with the VH and CH1 of the first polypeptide chain form a Fab that binds the cytokine (e.g., IL-2, IL-12 subunit (i.e., p35, p40, or the p35p40 heterodimeric complex), or IFN) polypeptide.

Compounds 1, 2, 3 and 4 are specific examples of inducible IL-2 prodrugs that comprise two polypeptide chains for use according to this disclosure. Compounds 1, 2, 3, and 4 and additional details regarding their activity is disclosed in WO2021/097376.

TABLE 1
Inducible IL-2 prodrugs

| IL-2 Prodrug | First Polypeptide | Second Polypeptide |
|---|---|---|
| Compound 1 | SEQ ID NO: 1 | SEQ ID NO: 5 |
| Compound 2 | SEQ ID NO: 2 | SEQ ID NO: 5 |
| Compound 3 | SEQ ID NO: 3 | SEQ ID NO: 5 |
| Compound 4 | SEQ ID NO: 4 | SEQ ID NO: 5 |

Cytokines that comprise two subunits, such as IL-12, can also comprise two or more different polypeptides. For instance, the first polypeptide can comprise an IL-12 subunit, and optionally a blocking element. The blocking element when present can be operably linked to the IL-12 subunit through a first protease cleavable linker. The second polypeptide chain can comprise an IL-12 subunit operably linked to a half-life extension element through a second protease cleavable linker, and optionally a IL-12 blocking element. The IL-12 blocking element when present can be operably linked to the IL-12 subunit through a protease cleavable linker or can be operably linked to the half-life extension element through a linker that is optionally protease cleavable. Only one of the first and second polypeptide contains the IL-12 blocking element. When the IL-12 subunit in the first polypeptide is p35, the IL-12 subunit in the second polypeptide is p40, and when the IL-12 subunit in the first polypeptide is p40, the IL-12 subunit in the second polypeptide is p35. A blocking element can be a single chain antibody that binds IL-12 or an antigen binding fragment thereof. The cleavable linkers in this complex can be the same or different.

The inducible cytokine polypeptide prodrug can comprise three different polypeptides. For example, one polypeptide chain comprises either the p35 or p40 IL-12 subunit, but not both, and a second polypeptide comprises the other IL-12 subunit and the third polypeptide comprises at least a portion (component) of the blocking element. The first polypeptide can comprise an IL-12 subunit, and optionally a half-life extension element. The half-life extension element when present can be operably linked to the IL-12 subunit through a protease cleavable linker.

The second polypeptide can comprise a IL-12 subunit, at least an antigen binding portion of an antibody light chain or an antigen binding portion of an antibody heavy chain, and optionally a half-life extension element. When the half-life extension element is present, it can be operably linked to the IL-12 subunit through a protease cleavable linker and the antibody heavy chain or light chain is either a) operably linked to the IL-12 subunit through a second protease cleavable linker, or b) operably linked to the half-life extension element through an optionally cleavable linker.

The third polypeptide can comprise can an antigen binding portion of an antibody heavy chain that is complementary to the light chain in the second polypeptide, or an antibody light chain that is complementary to the heavy chain in the second polypeptide and together with said light chain forms an IL-12 binding site. When the IL-12 subunit in the first polypeptide is p35, the IL-12 subunit in the second polypeptide is p40, and when the IL-12 subunit in the first polypeptide is p40, the IL-12 subunit in the second polypeptide is p35. In this complex, the IL-12 blocking element is preferably an antigen binding fragment of an antibody. The antigen binding fragment comprises as separate components, at least an antigen-binding portion of an antibody light chain and at least an antigen-binding portion of a complementary antibody heavy chain. The protease cleavable linkers in this inducible IL-12 polypeptide complex can be the same or different.

The inducible polypeptide complex can comprise two different polypeptides wherein p35 and p40 are located on the same polypeptide chain. A first polypeptide chain can comprise p35, p40, a half-life extension element and at least an antigen binding portion of an antibody light chain. p35 and p40 can be operably linked, and the half-life extension element can be operably linked to p40 through a first protease cleavable linker and the antigen binding portion of an antibody light chain can be operably linked to p35 through a protease cleavable linker.

Alternatively, the half-life extension element can be operably linked to p35 through a protease cleavable linker and the antigen binding portion of an antibody light chain is operably linked to p40 through a protease cleavable linker. The second polypeptide comprises at least an antigen binding portion of an antibody heavy chain that is complementary to the light chain in the second polypeptide and together with said light chain forms and IL-12 binding site. The protease cleavable linkers in this complex can be the same or different.

In an alternative format, a first polypeptide chain can comprise p35, p40, a half-life extension element and at least an antigen binding portion of an antibody heavy chain. p35 and p40 can be operably linked, and the half-life extension element can be operably linked to p40 or through a protease cleavable linker and the antigen binding portion of an antibody heavy chain can be operably linked to p35 through a protease cleavable linker. Alternatively, the half-life extension element can be operably linked to p35 through a protease cleavable linker and the antigen binding portion of an antibody heavy chain can be operably linked to p40 through a second protease cleavable linker. A second polypeptide comprises at least an antigen binding portion of an antibody light chain that is complementary to the heavy chain in the second polypeptide and together with said light chain forms and IL-12 binding site. The protease cleavable linkers in this complex can be the same or different.

In an example, the IL-12 polypeptide complex comprises a first polypeptide does not comprise a blocking element and the second polypeptide has the formula: [A]-[L1]-[B]-[L3]-[D] or [D]-[L3]-[B]-[L1]-[A] or [B]-[L1]-[A]-[L2]-[D] or [D]-[L1]-[A]-[L2]-[B], wherein, A is the IL-12 subunit; L1 is the first protease-cleavable linker; L2 is the second protease cleavable linker; L3 is the optionally cleavable linker; B is the half-life extension element; and D is the blocking element.

In another example, the first polypeptide comprises the formula: [A]-[L1]-[D] or [D]-[L1]-[A]; and the second polypeptide has the formula: [A′]-[L2]-[B] or [B]-[L2]-[A′], wherein A is either p35 or p40, wherein when A is p35, A′ is p40 and when A is p40, A′ is p35; A′ is either p35 or p40; L1 is the first protease cleavable linker; L2 is the second protease cleavable linker; B is the half-life extension element; and D is the blocking element.

Compounds 5, 6, 7, 8, 9, and 10 are specific examples of inducible IL-12 prodrugs that comprise two polypeptide chains for use according to this disclosure. Compounds 5, 6, 7, 8, 9, and 10 and additional details regarding their activity is disclosed in International Application No.: PCT/US2021/33014.

TABLE 2
Inducible IL-12 prodrugs

| IL-12 Prodrug | First Polypeptide | Second Polypeptide |
|---|---|---|
| Compound 5 | SEQ ID NO: 6 | SEQ ID NO: 12 |
| Compound 6 | SEQ ID NO: 7 | SEQ ID NO: 12 |
| Compound 7 | SEQ ID NO: 8 | SEQ ID NO: 13 |
| Compound 8 | SEQ ID NO: 9 | SEQ ID NO: 13 |
| Compound 9 | SEQ ID NO: 10 | SEQ ID NO: 13 |
| Compound 10 | SEQ ID NO: 11 | SEQ ID NO: 13 |

As described above, the cytokine can be a mutein, if desired. The cytokine mutein retains activity, for example intrinsic IL-12/IL-2/IFN receptor agonist activity.

### B. Half-Life Extension Element

The half-life extension element increases the in vivo half-life and provides altered pharmacodynamics and pharmacokinetics of the inducible cytokine prodrugs. Without being bound by theory, the half-life extension element alters pharmacodynamics properties including alteration of tissue distribution, penetration, and diffusion of the inducible cytokine prodrug. In some embodiments, the half-life extension element can improve tissue targeting, tissue penetration, diffusion within the tissue, and enhanced efficacy as compared with a protein without a half-life extension element. Without being bound by theory, an exemplary way to improve the pharmacokinetics of a polypeptide is by expression of an element in the polypeptide chain that binds to receptors that are recycled to the plasma membrane of cells rather than degraded in the lysosomes, such as the FcRn receptor on endothelial cells and transferrin receptor. Three types of proteins, e.g., human IgGs, HSA (or fragments), and transferrin, persist for much longer in human serum than would be predicted just by their size, which is a function of their ability to bind to receptors that are recycled rather than degraded in the lysosome. These proteins, or fragments retain FcRn binding and are routinely linked to other polypeptides to extend their serum half-life. HSA may also be directly bound to the pharmaceutical compositions or bound via a short linker. Fragments of HSA may also be used. HSA and fragments thereof can function as both a blocking element and a half-life extension element. Human IgGs and Fc fragments can also carry out a similar function.

The serum half-life extension element can also be antigen-binding polypeptide that binds to a protein with a long serum half-life such as serum albumin, transferrin and the like. Examples of such polypeptides include antibodies and fragments thereof including, a polyclonal antibody, a recombinant antibody, a human antibody, a humanized antibody a single chain variable fragment (scFv), single-domain antibody such as a heavy chain variable domain (VH), a light chain variable domain (VL) and a variable domain of camelid-type nanobody (VHH), a dAb and the like. Other suitable antigen-binding domain include non-immunoglobulin proteins that mimic antibody binding and/or structure such as, anticalins, affilins, affibody molecules, affimers, affitins, alphabodies, avimers, DARPins, fynomers, kunitz domain peptides, monobodies, and binding domains based on other engineered scaffolds such as SpA, GroEL, fibronectin, lipocallin and CTLA4 scaffolds. Further examples of antigen-binding polypeptides include a ligand for a desired receptor, a ligand-binding portion of a receptor, a lectin, and peptides that binds to or associates with one or more target antigens.

The half-life extension element as provided herein is preferably a human serum albumin (HSA) binding domain, and antigen binding polypeptide that binds human serum albumin or an immunoglobulin Fc or fragment thereof.

The half-life extension element of an inducible cytokine prodrug extends the half-life of the inducible cytokine prodrug by at least about two days, about three days, about four days, about five days, about six days, about seven days, about eight days, about nine days, about 10 days or more.

### C. Blocking Element

The blocking element can be any element that binds to the cytokine and inhibits the ability of the cytokine polypeptide to bind and activate its receptor. The blocking element can inhibit the ability of the cytokine (e.g. IL-2, IL-12, or IFN) to bind and/or activate its receptor e.g., by sterically blocking and/or by noncovalently binding to the cytokine polypeptide. The blocking element disclosed herein can bind to IL-2, IL-12 (e.g. p35, p40, or the heterodimer), or IFN (e.g., IFN-alpha (e.g., human IFN-alpha1, human IFN-alpha2, human IFN-alpha4, human IFN-alpha5, human IFN-alpha6, human IFN-alpha7, human IFN-alpha8, human IFN-alpha10, human IFN-alpha13, human IFN-alpha14, human IFN-alpha16, human IFN-alpha17, human IFN-alpha2) IFN-beta, IFN-gamma).

Examples of suitable blocking elements include the full length or a cytokine-binding fragment or mutein of the cognate receptor of a cytokine (e.g. IL-2, IL-12, or IFN). The cognate receptor for IL-2 can be the IL-2 alpha chain, the IL-2 beta chain, the IL-2 gamma chain, or combinations thereof. The cognate receptor for IL-12 can be IL-12Rβ1 and/or IL-12Rβ2. The cognate receptor for IFN can be the IFNGR receptor or a portion thereof. For instance, when the interferon polypeptide is an IFNalpha, such as INFalpha2a, the blocking element can be the extracellular portion of the INFalpha receptor 1 (IFNAR1) or interferon binding portion or mutein thereof, or the extracellular portion of the IFNalpha receptor 2 (IFNAR2) or interferon binding portion or mutein thereof. When the interferon polypeptide is IFNgamma, the blocking element can be the extracelluar portion of the IFNgamma receptor 1 (IFNGR1) or interferon binding portion or mutein thereof, or the extracellular portion of the IFNgamma receptor 2 (IFNGR2) or interferon binding portion or mutein thereof.

Antibodies and antigen-binding fragments thereof including, a polyclonal antibody, a recombinant antibody, a human antibody, a humanized antibody a single chain variable fragment (scFv), single-domain antibody such as a heavy chain variable domain (VH), a light chain variable domain (VL) and a variable domain of camelid-type nanobody (VHH), a dAb and the like that bind to a cytokine (e.g., IL-2, IL-12, or IFN) can also be used. Other suitable antigen-binding domain that bind to the cytokine polypeptide can also be used, include non-immunoglobulin proteins that mimic antibody binding and/or structure such as, anticalins, affilins, affibody molecules, affimers, affitins, alphabodies, avimers, DARPins, fynomers, kunitz domain peptides, monobodies, and binding domains based on other engineered scaffolds such as SpA, GroEL, fibronectin, lipocallin and CTLA4 scaffolds. Further examples of suitable blocking polypeptides include polypeptides that sterically inhibit or block binding of the to its cognate receptor. Advantageously, such moieties can also function as half-life extending elements. For example, a peptide that is modified by conjugation to a water-soluble polymer, such as PEG, can sterically inhibit or prevent binding of the cytokine to its receptor. Polypeptides, or fragments thereof, that have long serum half-lives can also be used, such as serum albumin (human serum albumin), immunoglobulin Fc, transferrin and the like, as well as fragments and muteins of such polypeptides.

Blocking elements that are particularly suitable are single chain variable fragments (scFv) or Fab fragments.

Also disclosed herein is an inducible cytokine prodrug e that contains a blocking element having specificity for a cytokine and further contains a half-life extension element.

The blocking element can contain two or more components that are present on the same polypeptide chain or on separate polypeptide chains. A first polypeptide chain can include an antibody light chain (VL+CL) or light chain variable domain (VL) and a second polypeptide can include an antibody heavy chain Fab fragment (VH+CH1) or heavy chain variable domain (VH) that is complementary to the VL+CL or VL on the first polypeptide. In such situations, these components can associate in the peptide complex to form an antigen-binding site, such as a Fab that binds the cytokine (e.g., IL-2, IL-12, IFN) and attenuates cytokine activity.

### D. Protease Cleavable Linker

As disclosed herein, the inducible cytokine prodrug comprises one or more linker sequences. A linker sequence serves to provide flexibility between the polypeptides, such that, for example, the blocking element is capable of inhibiting the activity of the cytokine. The linker can be located between the cytokine subunit, the half-life extension element, and/or the blocking element. As described herein the inducible cytokine prodrug comprises a protease cleavable linker. The protease cleavable linker can comprise one or more cleavage sites for one or more desired protease. Preferably, the desired protease is enriched or selectively expressed at the desired target site of the cytokine activity (e.g., the tumor microenvironment). Thus, the inducible cytokine prodrug is preferentially or selectively cleaved at the target site of desired cytokine activity.

Suitable linkers are typically less than about 100 amino acids. Such linkers can be of different lengths, such as from 1 amino acid (e.g., Gly) to 30 amino acids, from 1 amino acid to 40 amino acids, from 1 amino acid to 50 amino acids, from 1 amino acid to 60 amino acids, from 1 to 70 amino acids, from 1 to 80 amino acids, from 1 to 90 amino acids, and from 1 to 100 amino acids. In some embodiments, the linker is at least about 1, about 2, about 3, about 4, about 5, about 10, about 15, about 20, about 25, about 30, about 35, about 40, about 45, about 50, about 55, about 60, about 65, about 70, about 75, about 80, about 85, about 90, about 95, or about 100 amino acids in length. Preferred linkers are typically from about 5 amino acids to about 30 amino acids.

Preferably the lengths of linkers vary from 2 to 30 amino acids, optimized for each condition so that the linker does not impose any constraints on the conformation or interactions of the linked domain. In a preferred embodiment, the linker is cleavable by a cleaving agent, e.g., an enzyme. Preferably, the linker comprises a protease cleavage site. In some cases, the linker comprises one or more cleavage sites. The linker can comprise a single protease cleavage site. The linker can also comprise 2 or more protease cleavage sites. For example, 2 cleavage sites, 3 cleavage sites, 4, cleavage sites, 5 cleavage sites, or more. In cases the linker comprises 2 or more protease cleavage sites, the cleavage sites can be cleaved by the same protease or different proteases. A linker comprising two or more cleavage sites is referred to as a “tandem linker.” The two or more cleavage sites can be arranged in any desired orientation, including, but not limited tom one cleavage site adjacent to another cleavage site, one cleavage site overlapping another cleavage site, or one cleavage site following by another cleavage site with intervening amino acids between the two cleavage sites.

Of particular interest in the present invention are disease specific protease-cleavable linkers. Also preferred are protease-cleavable linkers that are preferentially cleaved at a desired location in the body, such as the tumor microenvironment, relative to the peripheral circulation.

For example, the rate at which the protease-cleavable linker is cleaved in the tumor microenvironment can be at least about 10 times, at least about 100 times, at least about 1000 times or at least about 10,000 times faster in the desired location in the body, e.g., the tumor microenvironment, in comparison to in the peripheral circulation (e.g., in plasma).

Proteases known to be associated with diseased cells or tissues include but are not limited to serine proteases, cysteine proteases, aspartate proteases, threonine proteases, glutamic acid proteases, metalloproteases, asparagine peptide lyases, serum proteases, cathepsins, Cathepsin B, Cathepsin C, Cathepsin D, Cathepsin E, Cathepsin G, Cathepsin K, Cathepsin L, kallikreins, hK1, hK10, hK15, plasmin, collagenase, Type IV collagenase, stromelysin, Factor Xa, chymotrypsin-like protease, trypsin-like protease, elastase-like protease, subtilisin-like protease, actinidain, bromelain, calpain, caspases, caspase-3, Mirl-CP, papain, HIV-1 protease, HSV protease, CMV protease, chymosin, renin, pepsin, matriptase, legumain, plasmepsin, nepenthesin, metalloexopeptidases, metalloendopeptidases, matrix metalloproteases (MMP), MMP1, MMP2, MMP3, MMP8, MMP9, MMP13, MMP11, MMPP14, urokinase plasminogen activator (uPA), enterokinase, prostate-specific antigen (PSA, hK3), interleukin-1l converting enzyme, thrombin, FAP (FAPa), dipeptidyl peptidase, meprins, granzymes and dipeptidyl peptidase IV (DPPIV/CD26). Proteases capable of cleaving linker amino acid sequences (which can be encoded by the chimeric nucleic acid sequences provided herein) can, for example, be selected from the group consisting of a prostate specific antigen (PSA), a matrix metalloproteinase (MMP), an A Disintigrin and a Metalloproteinase (ADAM), a plasminogen activator, a cathepsin, a caspase, a tumor cell surface protease, and an elastase. The MMP can, for example, be matrix metalloproteinase 2 (MMP2), matrix metalloproteinase 9 (MMP9), matrix metalloproteinase 14 (MMP14). In addition, or alternatively, the linker can be cleaved by a cathepsin, such as, Cathepsin B, Cathepsin C, Cathepsin D, Cathepsin E, Cathepsin G, Cathepsin K and/or Cathepsin L. Preferably, the linker can be cleaved by MMP14 or Cathepsin L.

Proteases useful for cleavage of linkers and for use in the inducible cytokine prodrug disclosed herein are presented in Table 3, and exemplary proteases and their cleavage site are presented in Table 4.

TABLE 3
Proteases relevant to inflammation and cancer

| | | |
|---|---|---|
| Protease | Specificity | Other aspects |

Secreted by killer T cells:

| | | |
|---|---|---|
| Granxyme B (grB) | Cleaves after Asp residues | Type of serine protease; strongly |
| | (asp-ase) | implicated in inducing perforin- |
| | | dependent target cell apoptosis |
| Granzyme A (grA) | trypsin-like, cleaves after | Type of serine protease; |
| | basic residues | |
| Granzyme H (grH) | Unknown substrate | Type of serine protease; |
| | specificity | Other granzymes are also |
| | | secreted by killer T cells, |
| | | but not all are present in humans |
| Caspase-8 | Cleaves after Asp residues | Type of cysteine protease; plays |
| | | essential role in TCR-induced |
| | | cellular expansion-exact |
| | | molecular role unclear |
| Mucosa-associated | Cleaves after arginine | Type of cysteine protease; likely |
| lymphoid tissue | residues | acts both as a scaffold and |
| (MALT1) | | proteolytically active enzyme |
| | | in the CBM-dependent signaling |
| | | pathway |
| Tryptase | Target: angiotensin I, | Type of mast cell-specific serine |
| | fibrinogen, prourokinase, | protease; trypsin-like; resistant |
| | TGFβ; preferentially | to inhibition by macromolecular |
| | cleaves proteins after | protease inhibitors expressed in |
| | lysine or arginine | mammals due to their tetrameric |
| | residues | structure, with all sites facing |
| | | narrow central pore; also |
| | | associated with inflammation |

Associated with inflammation:

| | | |
|---|---|---|
| Thrombin | Targets: FGF-2, | Type of serine protease; modulates |
| | HB-EGF, Osteo-pontin, | activity of vascular growth factors, |
| | PDGF, VEGF | chemokines and extracellular |
| | | proteins; strengthens VEGF- |
| | | induced proliferation; induces cell |
| | | migration; angiogenic factor; |
| | | regulates hemostasis |
| Chymase | Exhibit chymotrypsin-like | Type of mast cell-specific serine |
| | specificity, cleaving proteins | protease |
| | after aromatic amino acid | |
| | residues | |
| Carboxypeptidase | Cleaves amino acid residues | Type of zinc-dependent |
| A (MC-CPA) | from C-terminal end of | metalloproteinase |
| | peptides and proteins | |
| Kallikreins | Targets: high molecular | Type of serine protease; modulate |
| | weight kininogen, | relaxation response; contribute |
| | pro-urokinase | to inflammatory response; fibrin |
| | | degradation |
| Elastase | Targets: E-cadherin, GM- | Type of neutrophil serine protease; |
| | CSF, IL-1, IL-2, IL-6, IL-8, | degrades ECM components; |
| | p38^{MAPK}, TNFα, VE- | regulates inflammatory response; |
| | cadherin | activates pro-apoptotic signaling |
| Cathepsin G | Targets: EGF, ENA-78, | Type of serine protease; degrades |
| | IL-8, MCP-1, MMP-2, MT1- | ECM components; chemo- |
| | MMP, PAI-1, RANTES, | attractant of leukocytes; regulates |
| | TGFβ, TNFα | inflammatory response; promotes |
| | | apoptosis |
| PR-3 | Targets: ENA-78, IL-8, | Type of serine protease; promotes |
| | IL-18, JNK, p38^{MAPK}, | inflammatory response; activates |
| | TNFα | pro-apoptotic signaling |
| Granzyme M | Cleaves after Met and other | Type of serine protease; only |
| (grM) | long, unbranched | expressed in NK cells |
| | hydrophobic residues | |
| Calpains | Cleave between Arg and Gly | Family of cysteine proteases; |
| | | calcium-dependent; activation |
| | | is involved in the process of |
| | | numerous inflammation-associated |
| | | diseases |

TABLE 4
Exemplary Proteases and Protease
Recognition Sequences

| | Cleavage | SEQ |
|---|---|---|
| | Domain | ID |
| Protease | Sequence | NO: |
| MMP7 | KRALGLPG | 375 |
| MMP7 | (DE)₈RPLALW | 376 |
| | RS(DR)₈ | |
| MMP9 | PR(S/T)(L/I) | |
| | (S/T) | |
| MMP9 | LEATA | 378 |
| MMP11 | GGAANLVRGG | 379 |
| MMP14 | SGRIGFLRTA | 380 |
| MMP | PLGLAG | 381 |
| MMP | PLGLAX | 382 |
| MMP | PLGC(me)AG | 383 |
| MMP | ESPAYYTA | 384 |
| MMP | RLQLKL | 385 |
| MMP | RLQLKAC | 386 |
| MMP2, MMP9, MMP14 | EP(Cit)G | 387 |
| | (Hof)YL | |
| Urokinase plasminogen | SGRSA | 388 |
| activator (uPA) | | |
| Urokinase plasminogen | DAFK | 389 |
| activator (uPA) | | |
| Urokinase plasminogen | GGGRR | 390 |
| activator (uPA) | | |
| Lysosomal Enzyme | GFLG | 391 |
| Lysosomal Enzyme | ALAL | 392 |
| Lysosomal Enzyme | FK | |
| Cathepsin B | NLL | |
| Cathepsin D | PIC(Et)FF | 395 |
| Cathepsin K | GGPRGLPG | 396 |
| Prostate Specific Antigen | HSSKLQ | 397 |
| Prostate Specific Antigen | HSSKLQL | 398 |
| Prostate Specific Antigen | HSSKLQEDA | 399 |
| Herpes Simplex Virus | LVLASSSFGY | 400 |
| Protease | | |
| HIV Protease | GVSQNYPIVG | 401 |
| CMV Protease | GVVQASCRLA | 402 |
| Thrombin | F(Pip)RS | |
| Thrombin | DPRSFL | 404 |
| Thrombin | PPRSFL | 405 |
| Caspase-3 | DEVD | 406 |
| Caspase-3 | DEVDP | 407 |
| Caspase-3 | KGSGDVEG | 408 |
| Interleukin 1β | GWEHDG | 409 |
| converting enzyme | | |
| Enterokinase | EDDDDKA | 410 |
| FAP | KQEQNPGST | 411 |
| Kallikrein 2 | GKAFRR | 412 |
| Plasmin | DAFK | 413 |
| Plasmin | DVLK | 414 |
| Plasmin | DAFK | 415 |
| TOP | ALLLALL | 416 |
| | GPLGVRG | 417 |
| | IPVSLRSG | 418 |
| | VPLSLYSG | 419 |
| | SGESPAYYTA | 420 |

Exemplary protease cleavable linkers include, but are not limited to kallikrein cleavable linkers, thrombin cleavable linkers, chymase cleavable linkers, carboxypeptidase A cleavable linkers, cathepsin cleavable linkers, elastase cleavable linkers, FAP cleavable linkers, ADAM cleavable linkers, PR-3 cleavable linkers, granzyme M cleavable linkers, a calpain cleavable linkers, a matrix metalloproteinase (MMP) cleavable linkers, a plasminogen activator cleavable linkers, a caspase cleavable linkers, a tryptase cleavable linkers, or a tumor cell surface protease. Specifically, MMP9 cleavable linkers, ADAM cleavable linkers, CTSL1 cleavable linkers, FAPα cleavable linkers, and cathepsin cleavable linkers. Some preferred protease-cleavable linkers are cleaved by a MMP and/or a cathepsin.

The separation moieties disclosed herein are typically less than 100 amino acids. Such separation moieties can be of different lengths, such as from 1 amino acid (e.g., Gly) to 30 amino acids, from 1 amino acid to 40 amino acids, from 1 amino acid to 50 amino acids, from 1 amino acid to 60 amino acids, from 1 to 70 amino acids, from 1 to 80 amino acids, from 1 to 90 amino acids, and from 1 to 100 amino acids. In some embodiments, the linker is at least about 1, about 2, about 3, about 4, about 5, about 10, about 15, about 20, about 25, about 30, about 35, about 40, about 45, about 50, about 55, about 60, about 65, about 70, about 75, about 80, about 85, about 90, about 95, or about 100 amino acids in length. Preferred linkers are typically from about 5 amino acids to about 30 amino acids.

Preferably the lengths of linkers vary from 2 to 30 amino acids, optimized for each condition so that the linker does not impose any constraints on the conformation or interactions of the linked domains.

In some embodiments, the linker comprises the sequence

(SEQ ID NO: 195)
GPAGLYAQ
(SEQ ID NO: 196)
GPAGMKGL
(SEQ ID NO: 197)
PGGPAGIG
(SEQ ID NO: 198)
ALFKSSFP
(SEQ ID NO: 199)
ALFFSSPP
(SEQ ID NO: 200)
LAQRLRSS
(SEQ ID NO; 201)
LAQKLKSS
(SEQ ID NO: 202)
GALFKSSFPSGGGPAGLYAQGGSGKGGSGK
(SEQ ID NO: 203)
RGSGGGPAGLYAQGSGGGPAGLYAQGGSGK
(SEQ ID NO: 204)
KGGGPAGLYAQGPAGLYAQGPAGLYAQGSR
(SEQ ID NO: 205)
RGGPAGLYAQGGPAGLYAQGGGPAGLYAQK
(SEQ ID NO: 206)
KGGALFKSSFPGGPAGIGPLAQKLKSSGGS
(SEQ ID NO: 207)
SGGPGGPAGIGALFKSSFPLAQKLKSSGGG
(SEQ ID NO: 208)
RGPLAQKLKSSALFKSSFPGGPAGIGGGGK
(SEQ ID NO: 209)
GGGALFKSSFPLAQKLKSSPGGPAGIGGGR
(SEQ ID NO: 210)
RGPGGPAGIGPLAQKLKSSALFKSSFPGGG
(SEQ ID NO: 211)
RGGPLAQKLKSSPGGPAGIGALFKSSFPGK
(SEQ ID NO: 212)
RSGGPAGLYAQALFKSSFPLAQKLKSSGGG
(SEQ ID NO: 213)
GGPLAQKLKSSALFKSSFPGPAGLYAQGGR
(SEQ ID NO: 214)
GGALFKSSFPGPAGLYAQPLAQKLKSSGGK
(SEQ ID NO: 215)
RGGALFKSSFPLAQKLKSSGPAGLYAQGGK
(SEQ ID NO: 216)
RGGGPAGLYAQPLAQKLKSSALFKSSFPGG
(SEQ ID NO: 217)
SGPLAQKLKSSGPAGLYAQALFKSSFPGSK
(SEQ ID NO: 218)
KGGPGGPAGIGPLAQRLRSSALFKSSFPGR
(SEQ ID NO: 219)
KSGPGGPAGIGALFFSSPPLAQKLKSSGGR
or
(SEQ ID NO: 220)
SGGFPRSGGSFNPRTFGSKRKRRGSRGGGG

Certain preferred separation moieties comprises the sequence GPAGLYAQ (SEQ ID NO: 195) or ALFKSSFP (SEQ ID NO: 198). The separation moieties disclosed herein can comprise one or more cleavage motif or functional variants that are the same or different. The separation moieties can comprise 1, 2, 3, 4, 5, or more cleavage motifs or functional variants. Separation moieties comprising 30 amino acids can contain 2 cleavage motifs or functional variants, 3 cleavage motifs or functional variants or more. A “functional variant” of a linker retains the ability to be cleaved with high efficiency at a target site (e.g., a tumor microenvironment that expresses high levels of the protease) and are not cleaved or cleaved with low efficiency in the periphery (e.g., serum). For example, the functional variants retain at least about 50%, about 55%, about 60%, about 70%, about 80%, about 85%, about 95% or more of the cleavage efficiency of a linker comprising any one of SEQ ID NOs: 195-220 or 447-448.

The separation moieties comprising more than one cleavage motif can be selected from SEQ ID NOs: 195-201 or 447-448 and combinations thereof. Preferred separation moieties comprising more than one cleavage motif comprise the amino acids selected from SEQ ID NO: 202-220.

The linker can comprise both ALFKSSFP (SEQ ID NO: 198) and GPAGLYAQ (SEQ ID NO: 195). The linker can comprise two cleavage motifs that each have the sequence GPAGLYAQ (SEQ ID NO: 195). Alternatively or additionally, the linker can comprise two cleavage motifs that each have the sequence ALFKSSFP (SEQ ID NO: 198). The linker can comprise a third cleavage motif that is the same or different.

In some embodiments, the linker comprises an amino acid sequence that is at least about 90%, at least about 95%, at least about 96%, at least about 97%, at least about 98%, or at least 99% identical to SEQ ID NOs: 195 to SEQ ID NO: 220 or 447-448 over the full length of SEQ ID NO: 195-220 or SEQ ID NOS 447-448.

The disclosure also relates to functional variants of separation moieties comprising SEQ ID NOs: 195-220 or 447-448. The functional variants of separation moieties comprising SEQ ID NOs: 195-220 or 447-448 generally differ from SEQ ID NOs: 195-220 or 447-448 by one or a few amino acids (including substitutions, deletions, insertions, or any combination thereof), and substantially retain their ability to be cleaved by a protease.

The functional variants can contain at least one or more amino acid substitutions, deletions, or insertions relative to the separation moieties comprising SEQ ID NOs: 195-220 or 447-448. The functional variant can comprise 1, 2, 3, 4, 5, 6, 7, 8, 9, or 10 amino acid alterations comparted to the separation moieties comprising SEQ ID NOs: 195-220 or 447-448. In some preferred embodiments, the functional variant differs from the linker comprising SEQ ID NOs: 195-220 by less than 10, less, than 8, less than 5, less than 4, less than 3, less than 2, or one amino acid alterations, e.g., amino acid substitutions or deletions. In other embodiments, the functional variant may comprise 1, 2, 3, 4, 5, 6, 7, 8, 9, or 10 amino acid substitutions compared to SEQ ID NOs: 195-220 or 447-448. The amino acid substitution can be a conservative substitution or a non-conservative substitution, but preferably is a conservative substitution.

In other embodiments, the functional variants of the separation moieties may comprise 1, 2, 3, 4, or 5 or more non-conservative amino acid substitutions compared the separation moieties comprising SEQ ID NOs: 195-220 or 447-448. Non-conservative amino acid substitutions could be recognized by one of skill in the art. The functional variant of the linker preferably contains no more than 1, 2, 3, 4, or 5 amino acid deletions.

The amino acid sequences disclosed in the separation moieties can be described by the relative linear position in the linker with respect to the scissile bond. As will be well-understood by persons skilled in the art, separation moieties comprising 8 amino acid protease substrates (e.g., SEQ ID Nos: 195-201 or 447-448) contain amino acid at positions P4, P3, P2, P1, P1′, P2′, P3′, P4′, wherein the sessile bond is between P1 and P1′. For example, amino acid positions for the linker comprising the sequence GPAGLYAQ (SEQ ID NO: 195) can be described as follows:

| G | P | A | G | L | Y | A | Q |
|---|---|---|---|---|---|---|---|
| P4 | P3 | P2 | P1 | P1′ | P2′ | P3′ | P4′ |

“GPAGLYAQ” disclosed as SEQ ID NO: 195.

Amino acids positions for the linker comprising the sequence ALFKSSFP (SEQ ID NO: 198) can be described as follows:

| A | L | F | K | S | S | F | P |
|---|---|---|---|---|---|---|---|
| P4 | P3 | P2 | P1 | P1′ | P2′ | P3′ | P4′ |

“ALFKSSFP” disclosed as SEQ ID NO: 198.

Preferably, the amino acids surrounding the cleavage site (e.g., positions P1 and P1′ for SEQ ID NOs: 195-201 or 447-448) are not substituted.

In embodiments, the linker comprises the sequence GPAGLYAQ (SEQ ID NO: 195) or ALFKSSFP (SEQ ID NO: 198) or a functional variant of SEQ ID NO: 195 or a function variant of SEQ ID NO: 198. As described herein, a functional variant of PAGLYAQ (SEQ ID NO: 447) or ALFKSSFP (SEQ ID NO: 198) can comprise one or more amino acid substitutions, and substantially retain their ability to be cleaved by a protease. Specifically, the functional variants of GPAGLYAQ (SEQ ID NO: 195) is cleaved by MMP14, and the functional variant of ALFKSSFP (SEQ ID NO: 198) is cleaved by Capthepsin L (CTSL1). The functional variants also retain their ability to be cleaved with high efficiency at a target site (e.g., a tumor microenvironment that expresses high levels of the protease). For example, the functional variants of GPAGLYAQ (SEQ ID NO: 195) or ALFKSSFP (SEQ ID NO: 198) retain at least about 50%, about 55%, about 60%, about 70%, about 80%, about 85%, about 95% or more of the cleavage efficiency of a linker comprising amino acid sequence GPAGLYAQ (SEQ ID NO: 195) or ALFKSSFP (SEQ ID NO: 198), respectively.

Preferably, the functional variant of GPAGLYAQ (SEQ ID NO: 195) or ALFKSSFP (SEQ ID NO: 198) comprise no more than 1, 2, 3, 4, or 5 conservative amino acid substitutions compared to GPAGLYAQ (SEQ ID NO: 195) or ALFKSSFP (SEQ ID NO: 198). Preferably, the amino acids at position P1 and P1′ are not substituted. The amino acids at positions P1 and P1′ in SEQ ID NO: 195 are G and L, and the amino acids at positions P1 and P1′ in SEQ ID NO: 198 are K and S.

The functional variant of GPAGLYAQ (SEQ ID NO: 195) can preferably comprise one or more of the following: a) an arginine amino acid substitution at position P4, b) a leucine, valine, asparagine, or proline amino acid substitution at position P3, c) a asparagine amino acid substitution at position P2, d) a histidine, asparagine, or glycine amino acid substitution at position P1, e) a asparagine, isoleucine, or leucine amino acid substitution at position P1′, f) a tyrosine or arginine amino acid substitution at position P2′, g) a glycine, arginine, or alanine amino acid substitution at position P3′, h) or a serine, glutamine, or lysine amino acid substitution at position P4′. The following amino acid substitutions are disfavored in functional variants of GPAGLYAQ (SEQ ID NO: 195): a) arginine or isoleucine at position P3, b) alanine at position P2, c) valine at position P1, d) arginine, glycine, asparagine, or threonine at position P1′, e) aspartic acid or glutamic acid at position P2′, f) isoleucine at position P3′, g) valine at position P4′. In some embodiments, the functional variant of GPAGLYAQ (SEQ ID NO: 195) does not comprise an amino acid substitution at position P1 and/or P1′.

The amino acid substitution of the functional variant of GPAGLYAQ (SEQ ID NO: 195) preferably comprises an amino acid substitution at position P4 and/or P4′. For example, the functional variant of GPAGLYAQ (SEQ ID NO: 195) can comprise a leucine at position P4, or serine, glutamine, lysine, or phenylalanine at position P4. Alternatively or additionally, the functional variant of GPAGLYAQ (SEQ ID NO: 195) can comprise a glycine, phenylalanine, or a proline at position P4′.

In some embodiments, the amino acid substitutions at position P2 or P2′ of GPAGLYAQ (SEQ ID NO: 195) are not preferred.

In some embodiments, the functional variant of GPAGLYAQ (SEQ ID NO: 195) comprises the amino acid sequence selected from SEQ ID NOs: 221-295. Specific functional variants of GPAGLYAQ (SEQ ID NO: 195) include GPLGLYAQ (SEQ ID NO: 259), and GPAGLKGA (SEQ ID NO: 249).

The functional variants of LFKSSFP (SEQ ID NO: 448) preferably comprises hydrophobic amino acid substitutions. The functional variant of LFKSSFP (SEQ ID NO: 448) can preferably comprise one or more of the following: (a) lysine, histidine, serine, glutamine, leucine, proline, or phenylalanine at position P4; (b) lysine, histidine, glycine, proline, asparagine, phenylalanine at position P3; (c) arginine, leucine, alanine, glutamine, or histidine at position P2; (d) phenylalanine, histidine, threonine, alanine, or glutamine at position P1; (e) histidine, leucine, lysine, alanine, isoleucine, arginine, phenylalanine, asparagine, glutamic acid, or glycine at position P1′, (f) phenylalanine, leucine, isoleucine, lysine, alanine, glutamine, or proline at position P2′; (g) phenylalanine, leucine, glycine, serine, valine, histidine, alanine, or asparagine at position P3′; and phenylalanine, histidine, glycine, alanine, serine, valine, glutamine, lysine, or leucine.

The inclusion of aspartic acid and/or glutamic acid in functional variants of SEQ ID NO: 448 are generally disfavored and avoided. The following amino acid substitutions are also disfavored in functional variants of LFKSSFP (SEQ ID NO: 448): (a) alanine, serine, or glutamic acid at position P3; (b) proline, threonine, glycine, or aspartic acid at position P2; (c) proline at position P1; (d) proline at position P1′; (e) glycine at position P2′; (f) lysine or glutamic acid at position P3′; (g) aspartic acid at position P4′.

The amino acid substitution of the functional variant of LFKSSFP (SEQ ID NO: 448) preferably comprises an amino acid substitution at position P4 and/or P1. In some embodiments, an amino acid substitution of the functional variant of LFKSSFP (SEQ ID NO: 448) at position P4′ is not preferred.

In some embodiments, the functional variant of LFKSSFP (SEQ ID NO: 448) comprises the amino acid sequence selected from SEQ ID NOs: 296-374. Specific functional variants of LFKSSFP (SEQ ID NO: 448) include ALFFSSPP (SEQ ID NO: 199), ALFKSFPP (SEQ ID NO: 346), ALFKSLPP (SEQ ID NO: 347); ALFKHSPP (SEQ ID NO: 335); ALFKSIPP (SEQ ID NO: 348); ALFKSSLP (SEQ ID NO: 356); or SPFRSSRQ (SEQ ID NO: 297).

The separation moieties disclosed herein can form a stable prodrug under physiological conditions with the amino acid sequences (e.g. domains) that they link, while being capable of being cleaved by a protease. For example, the linker is stable (e.g., not cleaved or cleaved with low efficiency) in the circulation and cleaved with higher efficiency at a target site (i.e. a tumor microenvironment). Accordingly, fusion polypeptides that include the linkers disclosed herein can, if desired, have a prolonged circulation half-life and/or lower biological activity in the circulation in comparison to the components of the fusion polypeptide as separate molecular entities. Yet, when in the desired location (e.g., tumor microenvironment) the linkers can be efficiently cleaved to release the components that are joined together by the linker and restoring or nearly restoring the half-life and biological activity of the components as separate molecular entities.

The linker desirably remains stable in the circulation for at least 2 hours, at least 5, hours, at least 10 hours, at least 15 hours, at least 20 hours, at least 24 hours, at least 30 hours, at least 35 hours, at least 40 hours, at least 45 hours, at least 50 hours, at least 60 hours, at least 65 hours, at least 70 hours, at least 80 hours, at least 90 hours, or longer.

In some embodiments, the linker is cleaved by less than 90%, 80%, 70%, 60%, 50%, 40%, 30%, 20%, 20%, 5%, or 1% in the circulation as compared to the target location. The linker is also stable in the absence of an enzyme capable of cleaving the linker. However, upon expose to a suitable enzyme (i.e., a protease), the linker is cleaved resulting in separation of the linked domain.

### Additional Anti-Cancer Agents

This disclosure relates to a therapeutic combination of any of the inducible cytokine prodrugs disclosed herein (e.g., inducible cytokines that comprise an IL-2 polypeptide, and IL-12 polypeptide, or an IFN polypeptide) in combination with one or more additional agents to treat cancer (such as lymphoma), such as chemotherapeutic agents (e.g., cyclophosphamide, mechlorethamine, melphalan, chlorambucil, ifosfamide, busulfan, N-Nitroso-N-methylurea (MNU), carmustine (BCNU), lomustine (CCNU), semustine (MeCCNU), fotemustine, streptozotocin, dacarbazine, mitozolomide, temozolomide, thiotepa, mitomycin, diaziquone (AZQ), cisplatin, carboplatin, oxaliplatin, procarbazine, hexamethylmelamine, methotrexate, pemetrexed, fluorouracil (e.g. 5-fluorouracil), capecitabine, cytarabine, gemcitabine, decitabine, azacitidine, fludarabine, nelarabine, cladribine, clofarabine, pentostatin, thioguanine, mercaptopurine, vincristine, vinblastine, vinorelbine, vindesine, vinflunine, paclitaxel, docetaxel, etoposide, teniposide, doxorubicin, daunorubicin, epirubicin, idarubicin, pirarubicin, aclarubicin, mitoxantrone, actinomycin, bleomycin, bisantrene, gemcitabine, cytarabine, and the like), immuno-oncology agents and immune checkpoint inhibitors (e.g., anti-PD-L1, anti-CTLA4, anti-PD-1, anti-CD47, anti-GD2), oncolytic viruses and the like.

The inducible cytokine (e.g., IL-2, IL-12, and IFN) prodrugs disclosed herein can be combined with any desired additional anti-cancer agent. The inducible cytokine (e.g., IL-2, IL-12, and IFN) prodrugs disclosed herein can be combined with any desired anti-PD-1 antibody or any desired anti-PD-L1 antibody.

Exemplary anti-PD-1 antibodies that can be combined with the inducible cytokine prodrugs include, but are not limited to, AMP-224 (AstraZenica), 609A (3SBio), 704 (3SBio), 705 (3SBio), ABBV-181 (AbbVie), ADU-1503/bion-004 (Chinook Therapeutics), AGEN2034/balstilimab (Agenus), AK103 (Akeso), AK104 (Akeso), AK 112 (Akeso), AK123 (Akeso), AMG 256 (Amgen), AMG 404 (Amgen), ANB030 (AnaptysBio), ANKEBIO Anti-PD1 product (Anhui Anke Biotechnology), Anti PD-1/Anti-CD47 (DiNonA), ASKG915 (Ask Gene Pharmaceuticals), AV-MEL-1 (Aivita Biomedical), BCD-100 (Biocad CJSC), BI 754091 (Boehringer Ingelheim), BiCKI-IL-7 (OSE Immunotherapeutics), Boehringer-PD-1-unknown (Boehringer Ingelheim), BSK-050K01 (Biosion), Camrelizumab (Jiangsu Hengrui Medicine), CB201 (Crescendo Biologics), CB213 (Crescendo Biologics), CC-90006 (AnaptsBio), cetrelimab (J&J), chPD1 (Kiromic Biopharma), CMAB819 (Mabpharm), CS1003 (CStone Pharmaceuticals), CS17938 (Shenzhen Chipscreen Biosciences), CTX-8371 (Compass Therapeutics), CX-072 (CytomX Therapeutics), CX-188 (CytomX Therapeutics), cypalizumab (Harbin Gloria Pharmaceuticals), DB004 (DotBio), EMB02 (EpimAb Biotherapeutics), Geptanblimab/genolimzumab (Apollomics), GS19 (Suzhou Zelgen Biopharmaceuticals), HLX10 (Shanghai Henlius Biotech), HX008 (Taizhou HanZhong Pharmaceuticals), HY003 (Juventas Cell Therapy), IBI315/BH2950 (Innovent Biologics), IBI318 (Innovent Biologics), IBI319 (Innovent Biologics), IMM1802 (ImmuneOnco Biopharma), IMT200 (TrueBinding), Jemperli/dostarlimab (AnaptysBio), JTX-4014 (Jounce Therapeutics), Keytruda/pembrolizumab (Merck), LBL-006 (Nanjing Leads Biolabs), Libtayo/cemiplimab-rwlc (Regeneron Pharmaceuticals), LVGN3616 (Lyvgen Biopharma), LXF821 (Novartis), LY01015 (Luye Pharma Group), LY3462817 (Eli Lilly), MCLA-134 (Merus N.V.), MEDI5752 (AstraZenica), NIR178 (Novartis), ONCR-177 (Oncorus), ONO-4685 (Ono Pharmaceutical), Opdivo/nivolumab (Ono Pharmaceutical), MGD019 (MacroGenius), PD1-GDT CAR-T (Kiromic Biopharma), penpulimab (Akeso), PSB205 (Qilu Puget Sound Biotherapeutics), PT-001 (Merck), PT627 (Merck), RB-M1 (Refuge Biotechnologies), Retifanlimab (MacroGenics), RG6139 (Roche), RG6279 (Roche), RTX-002 (RubrYc Therapeutics), sasanlimab (Pfizer), Servier-PD1×LAG3-unknown (Servier), SL-279252/TAK-252 (Shattuck Labs), Sofusa anti-PD1 (Sorrento Therapeutics), spartalizumab (Novartis), SSI-361 (Lyvgen Biopharma), Sym021 (Servier), Tebotelimab (MacroGenics), tislelizumab (BeiGene), TSR-075 (AnaptsBio), Tuhura-DO/PD-1-unknown (Tuhura Biopharma), toripalimab (Shanghai Junshi Biosciences), sintilimab (Innovent Biologics), Unicar-CAR-T&PD-1-unknown (Shanghai Unicar-Therapy Bio-Medicine Technology), Xdivane (Xbrane Biopharma), XmAb20717 (Xencor), XmAb23104 (Xencor), YBL-006 (Y-Biologics), and zimberelimab (Arcus Biosciences).

The anti-PD-1 antibody that can be combined with the inducible cytokine prodrugs is typically an approved anti-PD-1 antibody. Approved anti-PD-1 antibodies include, but are not limited to, pembrolizumab (KEYTRUDA), dostarlimab (JEMPERLI), cemiplimab-rwlc (LIBATYO), nivolumab (OPDIVO), camrelizumab, tislelizumab, toripalimab, and sintilimab (TYVYT).

Exemplary anti-PD-L1 antibodies that can be combined with the inducible cytokine prodrugs include, but are not limited to, A167 (Sichuan Kelun), ABL501 (ABL Bio), ABL503 (ABL Bio), ABSK041 (Abbisko Therapeutics), ACE1708 (Acepodia), ACE-NK-PDL1 (Acepodia), ADG104 (Adagene), AK106 (Akeso), ALPN-202 (Alpine Immune Sciences), AN4005 (Adlai Nortye Biopharma), BMS-936559/MDX-1105 (BMS), APL-502/TQB2450 (Apollomics), Arbutus-PD-L1-unknown (Arbutus Biopharma), ASC22 (Ascletis Pharma), ATG-101 (Antengene), AVA-004 (Avacta Group), AVA021 (Avacta Group), AVA027 (Avacta Group), AVA-040-100 (Avacta Group), AVA04-Vbp (Avacta Group), Bavencio/avelumab (Merck), BCD-135 (Biocad CJSC), BGB-A333 (BeiGene), Bintrafusp alfa/GSK4045154 (Merck), CA-170/aupm-170 (Dr. Reddy's Laboratories), CCX559 (ChemoCentryx), CDR101 (CDR-Life), cosibelimab (Checkpoint Therapeutics), CTX-8371 (Compass Therapeutics), DiNonA-Solid Tumors-unknown (DiNonA), DR30207 (Zhejiang Doer Biologics), DuoBody-PD-L1×4-1BB (Ligand Pharmaceuticals), envafolimab (Alphamab Oncology), EPIM-001 (Elpis Biopharmaceuticals), ES101 (Elpiscience Biopharma), INBRX-105 (Inhibrx), FAZ053 (Novartis), FS118 (F-star Therapeutics), GB262 (Genor Biopharma), GS-4224 (Gilead), GT900008 (Kintor Pharmaceuticals), GX-P2 (Genexine), Hamni-PS-L1/CD47-Unknown (Hanmi Pharmaceutical), HBM7015 (HBM Holdings), HBM9167 (HBM Holdings), HLX20 (Shanghai Henlius Biotech), HTI-1088 (Jiangsu Hengrui Medicine), IBI318 (Innovent Biologics), IBI322 (Innovent Biologics), IBI323 (Innovent Biologics), IGM-7354 (IGM Biosciences), IMC-001 (Sorrento Therapeutics), Imfinzi/durvalumab (AstraZenica), IMM25 (ImmuneOnco Biopharma), IMM2502 (ImmuneOnco Biopharma), IMM2503 (ImmuneOnco Biopharma), IMM2504 (ImmuneOnco Biopharma), INCB86550 (Incyte), I0103 (IO Biotech), JS003 (Shanghai Junshi Biosciences), Jubilant-PD-L1-unknown (Jubilant Therapeutics), KD033 (Kadmon Holdings), KN046 (Alphamab Oncology), KY1003 (Sanofi), KY1043 (Sanofi), LY3300054 (Eli Lilly), LY3415244 (Eli Lilly), MRNA-6981 (Moderna), MSB2311 (Transcenta Holding), MT-6035 (Molecular Templates), ND021/NM21-1480 (Numab Therapeutics), OXOOIR (Oxford BioTherapeutics), PD-L1 based BsAbs (I-Mab), PD-L1 Boltbody ISAC (Bolt Biotherapeutics), PDL-GEX (Glycotope GmbH), PMC-122 (PharmAbcine), PMI06 (D&D Pharmatech), Protheragen-RV-scFv-PDL1-unknown (Protheragen), PRS-344 (Pieris Pharmaceuticals), Q-1802 (Merck), RC98 (Yantai Rongchang Pharmaceutical), RV-scFv-PDL1 (Protheragen), SenI_TAAx22P (Hebei Senlang Biotechnology), SHC020 (Nanjing Sanhome Pharmaceutical), sugemalimab (Ligand Pharmaceuticals), atezolizumab (Roche), TST005 (Transcenta Holding), TT-O1 (Topmunnity Therapeutics), TTX-siPDL1 (TransCode Therapeutics), UniCAR-T-PD-L1 (GEMoaB monoclonals), Vaximm (VXM10), and YBL-013 (Y-Biologics).

The anti-PD-L1 antibody that can be combined with the inducible cytokine prodrugs is typically an approved anti-PD-L1 antibody. Approved anti-PD-1 antibodies include, but are not limited to, avelumab (BAVENCIO), durvalumab (IMFINZI), and atezolizumab (TECENTRIQ).

### E. Therapy and Pharmaceutical Compositions

This disclosure relates to methods for treating cancer (lymphoma) using an inducible cytokine prodrug (e.g., IL-2, IL-12, or IFN), and to pharmaceutical compositions for use in such methods, including pharmaceutical compositions that contain an inducible cytokine prodrug (e.g., IL-2, IL-12, or IFN) in combination an excipient as described herein.

This disclosure further relates to methods for treating cancer (lymphoma) using a combination therapy comprising an inducible cytokine prodrug (e.g., IL-2, IL-12, or IFN) in combination with one or more additional agents to treat cancer (such as lymphoma) as disclosed herein, and to pharmaceutical compositions for use in such methods, including pharmaceutical compositions that contain an inducible cytokine prodrug (e.g., IL-2, IL-12, or IFN) in combination an one or more additional agents to treat cancer (such as lymphoma) as described herein.

This disclosure further relates to methods for treating cancer using a combination therapy comprising an inducible cytokine prodrug (e.g., IL-2, IL-12, or IFN) in combination with an anti-PD-1 antibody or an anti-PD-L1 antibody as disclosed herein, and to pharmaceutical compositions for use in such methods, including pharmaceutical compositions that contain an inducible cytokine prodrug (e.g., IL-2, IL-12, or IFN) in combination an anti-PD-1 antibody or an anti-PD-L1 antibody.

The disclosure relates to methods for treating cancer comprising administering to a subject in need thereof an effective amount of a combination therapy that includes an inducible cytokine prodrug and an anti-PD-1 antibody or an anti-PD-L1 antibody. The inducible an inducible cytokine prodrug and an anti-PD-1 antibody or an anti-PD-L1 antibody are administered to the subject so that there is overlap of the pharmacological activities of the two therapeutic agents. Accordingly, the inducible cytokine prodrug can be administered before, after, concurrently, or periprocedurally with an anti-PD-1 antibody or an anti-PD-L1 antibody. In some practices of the methods, an anti-PD-1 antibody or an anti-PD-L1 antibody is administered before the inducible cytokine prodrug. In some practices of the methods, the anti-PD-1 antibody or the anti-PD-L1 antibody is administered, then after administration is completed, the inducible cytokine prodrug is administered.

The inducible cytokine prodrug and the anti-PD-1/anti-PD-L1 antibody are typically administered systemically, for example by intravenous injection or preferably intravenous infusion. Other types of administration can be used, such as orally, parenterally, intravenous, intravenously, intra-articularly, intraperitoneally, intramuscularly, subcutaneously, intracavity, transdermally, intrahepatically, intracranially, nebulization/inhalation, by installation via bronchoscopy, or intratumorally.

The methods and compositions disclosed herein can be used to treat any suitable cancer, in particular solid tumors, such as sarcomas and carcinomas. For examples, the methods and compositions disclosed herein can be used to treat acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

In certain embodiments, the methods and compositions disclosed herein can be used to treat adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, mycosis fungoides, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, non-Hodgkin lymphoma, squamous carcinoma of the head and neck, malignant pleural mesothelioma, and Wilms tumor.

In certain preferred embodiments, the methods and compositions disclosed herein are used to treat melanoma, non-small cell lung cancer (NSCLC), small cell lung cancer (SCLC), head and neck squamous cell cancer (HNSCC), classical Hodgkin lymphoma (cHL), primary mediastinal large B cell lymphoma (PMBCL), urothelial carcinoma, microsatellite instability high or mismatch repair deficient cancer, microsatellite instability high or mismatch repair deficient colorectal cancer, gastric cancer, esophageal cancer, cervical cancer, hepatocellular carcinoma (HCC), merkel cell carcinoma (MCC), renal cell carcinoma (RCC), endometrial carcinoma, tumor mutational burden high cancer, cutaneous squamous cell carcinoma (cSCC), triple negative breast cancer (TNBC), urothelial carcinoma, colorectal cancer or oesophageal carcinoma.

In certain preferred embodiments, the methods and compositions disclosed herein are used to treat Merkel cell carcinoma (MCC), urothelial carcinoma (UC), renal cell carcinoma (RCC), non-small cell lung cancer (NSCLC), small cell lung cancer (SCLC), triple negative breast cancer (TNBC), endometrial cancer, cutaneous squamous cell carcinoma (CSCC), basal cell carcinoma (BCC), melanoma, malignant pleural mesothelioma, classical Hodgkin lymphoma (cHL), squamous cell carcinoma of the head and neck (SCCHN), hepatocellular carcinoma (HCC), esophageal squamous cell carcinoma (ESCC), non-squamous non-small cell lung cancer, or nasopharyngeal carcinoma (NPC).

Preferably, the methods and compositions disclosed herein are used to treat colon cancer, lung cancer, melanoma, renal cell carcinoma, or breast cancer.

In certain preferred embodiments, the methods and compositions disclosed herein are used to treat melanoma. As an example, the methods and compositions disclosed herein can be used to treat melanoma in subjects with unresectable or metastatic melanoma. As another example, the methods and compositions disclosed herein can be used for the adjuvant treatment of subjects with melanoma with involvement of lymph node(s) following complete resection.

In certain preferred embodiments, the methods and compositions disclosed herein are used to treat non-small cell lung cancer (NSCLC). As an example, the methods and compositions disclosed herein can be used to treat NSCLC in subjects with NSCLC expressing PD-L1 (e.g., Tumor Proportion Score (TPS)≥1%) as determined by an FDA-approved test, with no EGFR or ALK genomic tumor aberrations, and is: stage III where subjects are not candidates for surgical resection or definitive chemoradiation, or metastatic. As another example, the methods and compositions disclosed herein can be used to treat NSCLC in patients with metastatic NSCLC whose tumors express PD-L1 (TPS≥1%) as determined by an FDA-approved test, with disease progression on or after platinum-containing chemotherapy. As another example, the methods and compositions disclosed herein can be used in combination with pemetrexed and platinum chemotherapy, as first-line treatment of patients with metastatic nonsquamous NSCLC, with no EGFR or ALK genomic tumor aberrations. As another example, the methods and compositions disclosed herein can be used in combination with carboplatin and either paclitaxel or paclitaxel protein-bound, as first-line treatment of patients with metastatic squamous NSCLC.

In certain preferred embodiments, the methods and compositions disclosed herein are used to treat SCLC. As an example, the methods and compositions disclosed herein can be used to treat SCLC in subjects with metastatic SCLC with disease progression on or after platinum-based chemotherapy and at least one other prior line of therapy.

In certain preferred embodiments, the methods and compositions disclosed herein are used to treat HNSCC. As an example, the methods and compositions disclosed herein can be used to treat HNSCC in subjects with metastatic or with unresectable, recurrent HNSCC whose tumors express PD-L1 (e.g., Combined Positive Score (CPS)≥1) as determined by an FDA-approved test. As another example, the methods and compositions disclosed herein can be used to treat HNSCC in subjects with recurrent or metastatic HNSCC with disease progression on or after platinum-containing chemotherapy. As another example, the methods and compositions disclosed herein can be used in combination with platinum and fluorouracil for the first-line treatment of patients with metastatic or with unresectable, recurrent HNSCC.

In certain preferred embodiments, the methods and compositions disclosed herein are used to treat cHL. As an example, the methods and compositions disclosed herein can be used to treat cHL in subjects with relapsed or refractory cHL. As another example, the methods and compositions disclosed herein can be used to treat cHL in pediatric subjects with refractory cHL, or cHL that has relapsed after 2 or more lines of therapy.

In certain preferred embodiments, the methods and compositions disclosed herein are used to treat PMBCL. As an example, the methods and compositions disclosed herein can be used to treat PMBCL in subjects with refractory PMBCL, or in subjects who have relapsed after 2 or more prior lines of therapy.

In certain preferred embodiments, the methods and compositions disclosed herein are used to treat urothelial carcinoma. As an example, the methods and compositions disclosed herein can be used to treat urothelial carcinoma in subjects with locally advanced or metastatic urothelial carcinoma who are not eligible for cisplatin-containing chemotherapy and whose tumors express PD-L1 (e.g., Combined Positive Score (CPS)≥10) as determined by an FDA-approved test, or in subjects who are not eligible for any platinum-containing chemotherapy regardless of PD-L1 status. As another example, the methods and compositions disclosed herein can be used to treat urothelial carcinoma in subjects with locally advanced or metastatic urothelial carcinoma who have disease progression during or following platinum-containing chemotherapy or within 12 months of neoadjuvant or adjuvant treatment with platinum-containing chemotherapy. As another example, the methods and compositions disclosed herein can be used to treat urothelial carcinoma in subjects with Bacillus Calmette-Guerin (BCG)-unresponsive, high-risk, non-muscle invasive bladder cancer (NMIBC) with carcinoma in situ (CIS) with or without papillary tumors who are ineligible for or have elected not to undergo cystectomy.

In certain preferred embodiments, the methods and compositions disclosed herein are used to treat Microsatellite Instability-High (MSI-H) or Mismatch Repair Deficient (dMMR) Cancer. As an example, the methods and compositions disclosed herein can be used to treat MSI-H or dMMR cancer in subjects with unresectable or metastatic MSI-H or dMMR cancer wherein the solid tumors have progressed following prior treatment and the subject has no satisfactory alternative treatment options, or wherein the colorectal cancer has progressed following treatment with a fluoropyrimidine, oxaliplatin, and irinotecan.

In certain preferred embodiments, the methods and compositions disclosed herein are used to treat Microsatellite Instability-High (MSI-H) or Mismatch Repair Deficient (dMMR) Colorectal Cancer. As an example, the methods and compositions disclosed herein can be used to treat MSI-H or dMMR colorectal cancer in subjects with unresectable or metastatic MSI-H or dMMR colorectal cancer.

In certain preferred embodiments, the methods and compositions disclosed herein are used to treat gastric cancer. As an example, the methods and compositions disclosed herein can be used to treat gastric cancer in subjects with recurrent locally advanced or metastatic gastric or gastroesophageal junction adenocarcinoma whose tumors express PD-L1 (e.g., Combined Positive Score (CPS)≥1) as determined by an FDA-approved test, with disease progression on or after 2 or more prior lines of therapy including fluoropyrimidine- and platinum-containing chemotherapy and if appropriate, HER2/neu-targeted therapy.

In certain preferred embodiments, the methods and compositions disclosed herein are used to treat esophageal cancer. As an example, the methods and compositions disclosed herein can be used to treat esophageal cancer in subjects with locally advanced or metastatic esophageal or gastroesophageal junction (GEJ) (e.g., tumors with epicenter 1 to 5 centimeters above the GEJ) carcinoma that is not amenable to surgical resection or definitive chemoradiation, in combination with platinum- and fluoropyrimidine-based chemotherapy. As another example, the methods and compositions disclosed herein can be used to treat esophageal cancer in subjects with locally advanced or metastatic esophageal or gastroesophageal junction (GEJ) (e.g., tumors with epicenter 1 to 5 centimeters above the GEJ) carcinoma that is not amenable to surgical resection or definitive chemoradiation, after one or more prior lines of systemic therapy for patients with tumors of squamous cell histology that express PD-L1 (CPS≥10) as determined by an FDA-approved test.

In certain preferred embodiments, the methods and compositions disclosed herein are used to treat cervical cancer. As an example, the methods and compositions disclosed herein can be used to treat cervical cancer in subjects with recurrent or metastatic cervical cancer with disease progression on or after chemotherapy whose tumors express PD-L1 (e.g., Combined Positive Score (CPS)≥1) as determined by an FDA-approved test.

In certain preferred embodiments, the methods and compositions disclosed herein are used to treat HCC. As an example, the methods and compositions disclosed herein can be used to treat HCC in subjects who have been previously treated with sorafenib.

In certain preferred embodiments, the methods and compositions disclosed herein are used to treat MCC. As an example, the methods and compositions disclosed herein can be used to treat MCC in subjects with recurrent locally advanced or metastatic MCC.

In certain preferred embodiments, the methods and compositions disclosed herein are used to treat RCC. As an example, the methods and compositions disclosed herein can be used in combination with axitinib, for the first-line treatment of patients with advanced RCC.

In certain preferred embodiments, the methods and compositions disclosed herein are used to treat endometrial carcinoma. As an example, the methods and compositions disclosed herein can be used in combination with lenvatinib, for the treatment of subjects with advanced endometrial carcinoma that is not MSI-H or dMMR, who have disease progression following prior systemic therapy and are not candidates for curative surgery or radiation.

In certain preferred embodiments, the methods and compositions disclosed herein are used to treat Tumor Mutational Burden-High (TMB-H) Cancer. As an example, the methods and compositions disclosed herein can be used to treat TMB-H cancer in subjects with unresectable or metastatic tumor mutational burden-high (e.g., ≥10 mutations/megabase (mut/Mb)) solid tumors, as determined by an FDA-approved test, that have progressed following prior treatment and who have no satisfactory alternative treatment options.

In certain preferred embodiments, the methods and compositions disclosed herein are used to treat Cutaneous Squamous Cell Carcinoma (cSCC). As an example, the methods and compositions disclosed herein can be used to treat cSCC in subjects with recurrent or metastatic cutaneous squamous cell carcinoma that is not curable by surgery or radiation.

In certain preferred embodiments, the methods and compositions disclosed herein are used to treat Triple-Negative Breast Cancer (TNBC). As an example, the methods and compositions disclosed herein can be used in combination with chemotherapy, for the treatment of subjects with locally recurrent unresectable or metastatic TNBC whose tumors express PD-L1 (e.g., Combined Positive Score (CPS)≥10) as determined by an FDA approved test.

In certain preferred embodiments, the methods and compositions disclosed herein can be used to treat Merkel cell carcinoma (MCC). As an example, a combination comprising Avelumab can be used to treat MCC in subjects with metastatic MCC.

In certain preferred embodiments, the methods and compositions disclosed herein can be used to treat Urothelial Carcinoma (UC). As an example, a combination comprising avelumab can be used to treat UC in subjects with locally advanced or metastatic UC who have disease progression during or following platinum-containing chemotherapy. As another example, a combination comprising avelumab can be used to treat UC in subjects with locally advanced or metastatic UC who have disease progression within 12 months of neoadjuvant or adjuvant treatment with platinum-containing chemotherapy.

In certain preferred embodiments, the methods and compositions disclosed herein can be used to treat Renal Cell Carcinoma (RCC). As an example, a combination comprising avelumab and axitinib can be used in a subject with advanced RCC.

In certain preferred embodiments, the methods and compositions disclosed herein can be used to treat urothelial carcinoma (UC). As an example, a combination comprising Durvalumab can be used to treat UC in subjects with locally advanced or metastatic urothelial carcinoma who have disease progression during or following platinum-containing chemotherapy. As another example, a combination comprising Durvalumab can be used to treat UC in subjects with locally advanced or metastatic urothelial carcinoma who have disease progression within 12 months of neoadjuvant or adjuvant treatment with platinum-containing chemotherapy.

In certain preferred embodiments, the methods and compositions disclosed herein can be used to treat non-small cell lung cancer (NSCLC). As an example, a combination comprising Durvalumab can be used to treat NSCLC in subjects with unresectable, Stage III non-small cell lung cancer (NSCLC) whose disease has not progressed following concurrent platinum-based chemotherapy and radiation therapy.

In certain preferred embodiments, the methods and compositions disclosed herein can be used to treat small cell lung cancer (SCLC). As an example, a combination comprising Durvalumab can be used in combination with etoposide and either carboplatin or cisplatin, as first-line treatment of adult subjects with extensive-stage small cell lung cancer (ES-SCLC).

In certain preferred embodiments, the methods and compositions disclosed herein can be used to treat urothelial carcinoma (UC). As an example, a combination comprising Atezolizumab can be used to treat UC in adult subjects with locally advanced or metastatic urothelial carcinoma who are not eligible for cisplatin-containing chemotherapy and whose tumors express PD-L1 (e.g., PD-L1 stained tumor-infiltrating immune cells [IC] covering ≥5% of the tumor area), as determined by an FDA-approved test, or are not eligible for any platinum-containing chemotherapy regardless of PD-L1 status, or have disease progression during or following any platinum-containing chemotherapy, or within 12 months of neoadjuvant or adjuvant chemotherapy.

In certain preferred embodiments, the methods and compositions disclosed herein can be used to treat NSCLC. As an example, a combination comprising Atezolizumab can be used to treat NSCLC in adult subjects with metastatic NSCLC whose tumors have high PD-L1 expression (e.g., PD-L1 stained ≥50% of tumor cells [TC≥50%] or PD-L1 stained tumor-infiltrating immune cells [IC] covering ≥10% of the tumor area [IC≥10%]), as determined by an FDA-approved test, with no EGFR or ALK genomic tumor aberrations. As another example, a combination comprising Atezolizumab can be used in combination with bevacizumab, paclitaxel, and carboplatin, for the first-line treatment of adult subjects with metastatic non-squamous NSCLC with no EGFR or ALK genomic tumor aberrations. As another example, a combination comprising Atezolizumab can be used in combination with paclitaxel protein-bound and carboplatin for the first-line treatment of adult subjects with metastatic non-squamous NSCLC with no EGFR or ALK genomic tumor aberrations. As another example, a combination comprising Atezolizumab can be used to treat NSCLC in adult subjects with metastatic NSCLC who have disease progression during or following platinum-containing chemotherapy.

In certain preferred embodiments, the methods and compositions disclosed herein can be used to treat triple negative breast cancer (TNBC). As an example, a combination comprising Atezolizumab can be used in combination with paclitaxel protein-bound for the treatment of adult subjects with unresectable locally advanced or metastatic TNBC whose tumors express PD-L1 (e.g., PD-L1 stained tumor-infiltrating immune cells [IC] of any intensity covering ≥1% of the tumor area), as determined by an FDA approved test.

In certain preferred embodiments, the methods and compositions disclosed herein can be used to treat Small cell lung cancer (SCLC). As an example, a combination comprising Atezolizumab can be used in combination with carboplatin and etoposide, for the first-line treatment of adult subjects with extensive-stage small cell lung cancer (ES-SCLC).

In certain preferred embodiments, the methods and compositions disclosed herein can be used to treat endometrial cancer. As an example, a combination comprising Dostarlimab can be used to treat endometrial cancer in adult subjects with mismatch repair deficient (dMMR) recurrent or advanced endometrial cancer, as determined by an FDA-approved test, that has progressed on or following prior treatment with a platinum-containing regimen.

In certain preferred embodiments, the methods and compositions disclosed herein can be used to treat cutaneous squamous cell carcinoma (CSCC). As an example, a combination comprising Cemiplimab-rwlc can be used to treat CSCC in subjects with metastatic cutaneous squamous cell carcinoma (mCSCC) or locally advanced CSCC (laCSCC) who are not candidates for curative surgery or curative radiation.

In certain preferred embodiments, the methods and compositions disclosed herein can be used to treat basal cell carcinoma (BCC). As an example, a combination comprising Cemiplimab-rwlc can be used to treat BCC in subjects with locally advanced BCC (laBCC) previously treated with a hedgehog pathway inhibitor or for whom a hedgehog pathway inhibitor is not appropriate.

In certain preferred embodiments, the methods and compositions disclosed herein can be used to treat NSCLC. As an example, a combination comprising Cemiplimab-rwlc can be used to treat NSCLC in subjects whose tumors have high PD-L1 expression (e.g., Tumor Proportion Score (TPS)≥50%) as determined by an FDA-approved test, with no EGFR, ALK or ROS1 aberrations, and is locally advanced where subjects are not candidates for surgical resection or definitive chemoradiation, or metastatic.

In certain preferred embodiments, the methods and compositions disclosed herein can be used to treat melanoma. As an example, a combination comprising Nivolumab can be used to treat melanoma in subjects with unresectable or metastatic melanoma, as a single agent or in combination with ipilimumab. As another example, a combination comprising Nivolumab can be used to treat melanoma in subjects with melanoma with lymph node involvement or metastatic disease who have undergone complete resection, in the adjuvant setting.

In certain preferred embodiments, the methods and compositions disclosed herein can be used to treat NSCLC. As an example, a combination comprising Nivolumab can be used to treat NSCLC in adult subjects with metastatic non-small cell lung cancer expressing PD-L1 (≥1%) as determined by an FDA-approved test, with no EGFR or ALK genomic tumor aberrations, as first-line treatment in combination with ipilimumab. As another example, a combination comprising NSCLC can be used to treat melanoma in adult subjects with metastatic or recurrent non-small cell lung cancer with no EGFR or ALK genomic tumor aberrations as first-line treatment, in combination with ipilimumab and 2 cycles of platinum-doublet chemotherapy. As another example, a combination comprising NSCLC can be used to treat melanoma in subjects with metastatic non-small cell lung cancer and progression on or after platinum-based chemotherapy.

In certain preferred embodiments, the methods and compositions disclosed herein can be used to treat malignant pleural mesothelioma. As an example, a combination comprising Nivolumab can be used to treat malignant pleural mesothelioma in adult subjects with unresectable malignant pleural mesothelioma, as first-line treatment in combination with ipilimumab.

In certain preferred embodiments, the methods and compositions disclosed herein can be used to treat RCC. As an example, a combination comprising Nivolumab can be used to treat RCC in subjects with intermediate or poor risk advanced renal cell carcinoma, as a first-line treatment in combination with ipilimumab. As another example, a combination comprising Nivolumab can be used to treat RCC in subjects with advanced renal cell carcinoma, as a first-line treatment in combination with cabozantinib. As another example, a combination comprising Nivolumab can be used to treat RCC in subjects with advanced renal cell carcinoma who have received prior anti-angiogenic therapy.

In certain preferred embodiments, the methods and compositions disclosed herein can be used to treat classical Hodgkin lymphoma (cHL). As an example, a combination comprising Nivolumab can be used to treat cHL in adult subjects with cHL that has relapsed or progressed after autologous hematopoietic stem cell transplantation (HSCT) and brentuximab vedotin, or 3 or more lines of systemic therapy that includes autologous HSCT.

In certain preferred embodiments, the methods and compositions disclosed herein can be used to treat squamous cell carcinoma of the head and neck (SCCHN). As an example, a combination comprising Nivolumab can be used to treat SCCHN in subjects with recurrent or metastatic squamous cell carcinoma of the head and neck with disease progression on or after a platinum-based therapy.

In certain preferred embodiments, the methods and compositions disclosed herein can be used to treat urothelial carcinoma (UC). As an example, a combination comprising Nivolumab can be used to treat UC in subjects with locally advanced or metastatic urothelial carcinoma who have disease progression during or following platinum-containing chemotherapy or have disease progression within 12 months of neoadjuvant or adjuvant treatment with platinum-containing chemotherapy.

In certain preferred embodiments, the methods and compositions disclosed herein can be used to treat colorectal cancer. As an example, a combination comprising Nivolumab can be used to treat colorectal cancer in subjects with microsatellite instability-high (MSI-H) or mismatch repair deficient (dMMR) metastatic colorectal cancer that has progressed following treatment with a fluoropyrimidine, oxaliplatin, and irinotecan, as a single agent or in combination with ipilimumab.

In certain preferred embodiments, the methods and compositions disclosed herein can be used to treat hepatocellular carcinoma (HCC). As an example, a combination comprising Nivolumab can be used to treat HCC in subjects with HCC who have been previously treated with sorafenib, as a single agent or in combination with ipilimumab.

In certain preferred embodiments, the methods and compositions disclosed herein can be used to treat esophageal squamous cell carcinoma (ESCC). As an example, a combination comprising Nivolumab can be used to treat ESCC in subjects with unresectable advanced, recurrent or metastatic esophageal squamous cell carcinoma after prior fluoropyrimidine- and platinum-based chemotherapy.

In certain preferred embodiments, a combination comprising Camrelizumab can be used to treat cHL.

In certain preferred embodiments, a combination comprising Tislelizumab can be used to treat non-squamous non-small cell lung cancer. In certain preferred embodiments, a combination comprising Tislelizumab can be used to treat hepatocellular carcinoma (HCC).

In certain preferred embodiments, a combination comprising Toripalimab can be used to treat urothelial carcinoma. In certain preferred embodiments, a combination comprising Toripalimab can be used to treat melanoma. In certain preferred embodiments, a combination comprising Toripalimab can be used to treat nasopharyngeal carcinoma (NPC).

In certain preferred embodiments, a combination comprising Sintilimab can be used to treat non-squamous non-small cell lung cancer. In certain preferred embodiments, a combination comprising Sintilimab can be used to treat cHL.

The cancer to be treated using the methods and compositions of this disclosure can be metastatic cancer. The methods and compositions disclosed herein can be used to treat metastatic renal clear cell carcinoma or metastatic cutaneous malignant melanoma.

In certain embodiments, the cancer is a lymphoma, such as Non-Hodgkin Lymphoma, Hodgkin Lymphoma, diffuse large B-cell lymphoma, primary mediastinal B cell lymphoma, follicular lymphoma, small lymphocytic lymphoma, chronic lymphocytic leukemia, marginal zone lymphoma, mantle cell lymphoma, Waldenstrom's macroglobulinemia, Burkitt lymphoma, peripheral T cell lymphoma, anaplastic large cell lymphoma, angioimmunoblastic lymphoma, cutaneous T cell lymphoma, central nervous system lymphoma, grey zone lymphoma, double hit lymphoma, triple hit lymphoma, high grade B cell lymphomas not otherwise specified, lymphoblastic lymphoma, lymphoplasmacytic lymphoma, MALT lymphoma, monocytoid B cell lymphoma, natural killer (NK) cell lymphoma, mycosis fungoides, Sezary syndrome, enteropathy-type T cell lymphoma, hepatosplenic gamma/delta T cell lymphoma, and the like.

If desired, additional therapeutic agents can be administered to the subject as described herein. Typically such additional therapeutic agents are anti-cancer agents such as chemotherapeutic agents (e.g., adriamycin, cerubidine, bleomycin, alkeran, velban, oncovin, fluorouracil, thiotepa, methotrexate, bisantrene, noantrone, thiguanine, cytaribine, procarabizine), other immune-check point inhibitors (e.g., anti-CTLA4 (ipilimumab (YERVOY)), other cytokines (such as IL-12, inducible IL-12 prodrugs, inducible IFN, inducible IFN prodrugs, IL-2 or IL-2 prodrugs), angiogenesis inhibitors, antibody-drug conjugates (e.g., trastuzumab emtansine (KADCYLA), trastuzumab deruxtecan (ENHERTU), enfortumab vedotin (PADCEV), sacituzumab govitecan (TRODELVY), cellular therapies (e.g., CAR-T, TCT-T, T-cell therapy, such as tumor infiltrating lymphocyte (TIL) therapy), oncolytic viruses, radiation therapy and/or small molecules.

An additional therapeutic agent can be, for example, pemetrexed, a platinum chemotherapeutic agent, carboplatin, paclitaxel, protein-bound paclitaxel, fluorouracil, a fluoropyrimidine-based chemotherapeutic agent, or axitinib.

This disclosure also relates to pharmaceutical compositions that contain an inducible cytokine prodrug and an anti-PD-1 or an anti-PD-L1 antibody, and to the use of such pharmaceutical compositions to treat cancer.

The pharmaceutical compositions can take a variety of forms, e.g., liquid, lyophilized, and typically contain a suitable pharmaceutically acceptable carrier. Pharmaceutically acceptable carriers (or excipients) are the non-active ingredient components of the pharmaceutical composition and are not biologically or otherwise undesirable, i.e., the material is administered to a subject without causing undesirable biological effects or interacting in a deleterious manner with the other components of the pharmaceutical formulation or composition in which it is contained. Carriers are frequently selected to minimize degradation of the active ingredient and to minimize adverse side effects in the subject.

Suitable carriers and their formulations are described in Remington: The Science and Practice of Pharmacy, 21st Edition, David B. Troy, ed., Lippicott Williams & Wilkins (2005). Examples of the pharmaceutically-acceptable carriers include, but are not limited to, sterile water, saline, buffered solutions like Ringer's solution, and dextrose solution. Other carriers include sustained release preparations such as semipermeable matrices of solid hydrophobic polymers containing the immunogenic polypeptides. Matrices are in the form of shaped articles, e.g., films, liposomes, or microparticles. Certain carriers may be more preferable depending upon, for instance, the route of administration and concentration of composition being administered. Carriers are those suitable for administration of the chimeric polypeptides or nucleic acid sequences encoding the chimeric polypeptides to humans or other subjects.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like. Typically, an appropriate amount of a pharmaceutically-acceptable salt is used in the formulation to render the formulation isotonic, although the formulation can be hypertonic or hypotonic if desired. The pH of the solution is generally about 5 to about 8 or from about 7 to 7.5.

Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

Compositions for oral administration include powders or granules, suspension or solutions in water or non-aqueous media, capsules, sachets, or tables. Thickeners, flavorings, diluents, emulsifiers, dispersing aids or binders are optionally desirable.

This disclosure also relates to a kit that includes a pharmaceutical composition that contains an a) inducible cytokine prodrug composition, for example as a liquid composition or a lyophilized composition, in a suitable container (e.g., a vial, bag or the like), and b) a pembrolizumab composition, for example as a liquid composition or a lyophilized composition, in a suitable container (e.g., a vial, bag or the like). The kit can further include other components, such as sterile water or saline for reconstitution of lyophilized compositions.

### F. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

“Cytokine” is a well-known term of art that refers to any of a class of immunoregulatory proteins (such as interleukin or interferon) that are secreted by cells especially of the immune system and that are modulators of the immune system. Cytokine polypeptides that can be used in the fusion proteins disclosed herein include, but are not limited to transforming growth factors, such as TGF-a and TGF-P (e.g., TGFbeta1, TGFbeta2, TGFbeta3); interferons, such as interferon-a, interferon-P, interferon-7, interferon-kappa and interferon-omega; interleukins, such as IL-1, IL-1a, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21 and IL-25; tumor necrosis factors, such as tumor necrosis factor alpha and lymphotoxin; chemokines (e.g., C—X—C motif chemokine 10 (CXCL10), CCL19, CCL20, CCL21), and granulocyte macrophage-colony stimulating factor (GM-CS), as well as fragments of such polypeptides that active the cognate receptors for the cytokine (i.e., functional fragments of the foregoing). “Chemokine” is a term of art that refers to any of a family of small cytokines with the ability to induce directed chemotaxis in nearby responsive cells.

As used herein, the terms “inducible” refer to the ability of a protein, i.e. IL-2, IL-12, or IFN, that is part of a prodrug, to bind its receptor and effectuate activity upon cleavage of the prodrug in the tumor microenvironment. The inducible cytokine prodrugs disclosed herein have attenuated or no cytokine agonist activity, but upon cleavage in the tumor microenvironment release active cytokine.

“Attenuated” activity, means that biological activity and typically cytokine (i.e., IL-2, IL-12 or IFN) agonist activity is decreased as compared to the activity of the natural cytokine (i.e., IL-2, IL-12 or IFN). The inducible cytokine prodrugs disclosed herein have attenuated cytokine receptor agonists activity, that is at least about 10×, at least about 50×, at least about 100×, at least about 250×, at least about 500×, at least about 1000× or less agonist activity as compared to natural cytokine (i.e., IL-2, IL-12 or IFN). Upon cleavage in the tumor microenvironment, cytokine is released that is active. Typically, the cytokine that is released has cytokine receptor agonist activity that is at least about 10×, at least about 50×, at least about 100×, at least about 250×, at least about 500×, or at least about 1000× greater than the IL-2 receptor activating activity of the prodrug.

As used herein, the terms “peptide”, “polypeptide”, or “protein” are used broadly to mean two or more amino acids linked by a peptide bond. Protein, peptide, and polypeptide are also used herein interchangeably to refer to amino acid sequences. It should be recognized that the term polypeptide is not used herein to suggest a particular size or number of amino acids comprising the molecule and that a peptide of the invention can contain up to several amino acid residues or more.

As used throughout, “subject” can be a vertebrate, more specifically a mammal (e.g. a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, “patient” or “subject” may be used interchangeably and can refer to a subject with a disease or disorder (e.g. cancer). The term patient or subject includes human and veterinary subjects.

As used herein the terms “treatment”, “treat”, or “treating” refers to a method of reducing the effects of a disease or condition or symptom of the disease or condition. Thus, in the disclosed methods, treatment can refer to at least about 10%, at least about 20%, at least about 30%, at least about 40%, at least about 50%, at least about 60%, at least about 70%, at least about 80%, at least about 90%, or substantially complete reduction in the severity of an established disease or condition or symptom of the disease or condition, such as reduction in tumor volume, reduction in tumor burden, reduction in death. For example, a method for treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms “prevent”, “preventing”, and “prevention” of a disease or disorder refers to an action, for example, administration of the chimeric polypeptide or nucleic acid sequence encoding the chimeric polypeptide, that occurs before or at about the same time a subject begins to show one or more symptoms of the disease or disorder, which inhibits or delays onset or exacerbation of one or more symptoms of the disease or disorder.

As used herein, references to “decreasing”, “reducing”, or “inhibiting” include a change of at least about 10%, of at least about 20%, of at least about 30%, of at least about 40%, of at least about 50%, of at least about 60%, of at least about 70%, of at least about 80%, of at least about 90% or greater as compared to a suitable control level. Such terms can include but do not necessarily include complete elimination of a function or property, such as agonist activity.

The term “sequence variant” refers to an amino acid sequence of a polypeptide that has substantially similar biological activity as a reference polypeptide but differs in amino acid sequence or to the nucleotide sequence of a nucleic acid that has substantially similar biological activity (e.g., encodes a protein with substantially similar activity) as a reference sequence but differs in nucleotide sequence. Typically the amino acid or nucleotide sequence of a “sequence variant” is highly similar (e.g. at least about 80% similar) to that of a reference sequence. Those of skill in the art readily understand how to determine the identity of two polypeptides or two nucleic acids. For example, the identity can be calculated after aligning the two sequences so that the identity is at its highest level over a defined number of nucleotides or amino acids. Optimal alignment of sequences for comparison may be conducted by the local identity algorithm of Smith and Waterman Adv. Appl. Math. 2:482 (1981), by the identity alignment algorithm of Needleman and Wunsch, J. Mol. Biol. 48:443 (1970), by the search for similarity method of Pearson and Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The term “conservative amino acid substitution” is a term of art that refers to the replacement of an amino acid in a polypeptide with another amino acid that has similar biochemical properties, such as size, charge and hydrophobicity as a reference amino acid. It is well-known that conservative amino acid replacements in the amino acid sequence of a polypeptide frequently do not significantly alter the overall structure or function of the polypeptide. Conservative substitutions of amino acids are known to those skilled in the art. Conservative substitutions of amino acids can include, but not limited to, substitutions made amongst amino acids within the following groups: (a) M, I, L, V; (b) F, Y, W; (c) K, R, H; (d) A, G; (e) S, T; (f) Q, N; and (g) E, D. For instance, a person of ordinary skill in the art reasonably expect that an isolated replacement of a leucine with an isoleucine or valine, an aspartate with a glutamate, a threonine with a serine, or a similar replacement of an amino acid with a structurally related amino acid will not have a major effect on the biological activity of the resulting molecule.

The term “effective amount,” as used herein, refers to the amount of agent (inducible cytokine prodrug and an anti-PD-1 or anti-PD-L1 antibody) that is administered to achieve the desired effect under the conditions of administration, such an amount that reduces tumor size, reduces tumor burden, extends progression free survival or extends overall survival. The actual effective amount selected will depend on the particular cancer being treated and its stage and other factors, such as the subject's age, gender, weight, ethnicity, prior treatments and response to those treatments and other factors. Suitable amounts of inducible cytokine prodrug and pembrolizumab to be administered, and dosage schedules for a particular patient can be determined by a clinician of ordinary skill based on these and other considerations.

Preferably, the methods and compositions disclosed herein are used to treat colon cancer, lung cancer, melanoma, renal cell carcinoma, breast cancer, squamous carcinoma of the head and neck.

In certain preferred embodiments, the methods and compositions disclosed herein are used to treat melanoma, non-small cell lung cancer (NSCLC), small cell lung cancer (SCLC), head and neck squamous cell cancer (HNSCC), classical Hodgkin lymphoma (cHL), primary mediastinal large B cell lymphoma (PMBCL), urothelial carcinoma, microsatellite instability high or mismatch repair deficient cancer, microsatellite instability high or mismatch repair deficient colorectal cancer, gastric cancer, esophageal cancer, cervical cancer, hepatocellular carcinoma (HCC), merkel cell carcinoma (MCC), renal cell carcinoma (RCC), endometrial carcinoma, tumor mutational burden high cancer, cutaneous squamous cell carcinoma (cSCC), triple negative breast cancer (TNBC), urothelial carcinoma, colorectal cancer or oesophageal carcinoma.

### 6. EQUIVALENTS

It will be readily apparent to those skilled in the art that other suitable modifications and adaptions of the methods of the invention described herein are obvious and may be made using suitable equivalents without departing from the scope of the disclosure or the embodiments.

Having now described certain compounds and methods in detail, the same will be more clearly understood by reference to the following examples, which are introduced for illustration only and not intended to be limiting.

### 7. EXAMPLES

The following are examples of methods and compositions of the invention. It is understood that various other embodiments may be practiced, given the general description provided herein.

### Example 1. CT26 Experiments—Treatment with ACP16 Alone or in Combination with Anti-PD1 Antibody

The CT26 cell line, a rapidly growing colon adenocarcinoma cell line that expresses MM4P9 in vitro, was used. Using this tumor model, the ability of fusion proteins to affect tumor growth was examined.

TABLE 5

| Gr. | N | Agent | Formulation dose | Route | Schedule |
|---|---|---|---|---|---|
| 1^{#} | 12 | vehicle 1// | na//na | ip//ip | days 1, 4, 8, 11// |
| | | vehicle 2 | | | days 3, 6, 10, 13 |
| 2 | 10 | vehicle 1// | na//70 ug/animal | ip//ip | days 1, 4, 8, 11// |
| | | ACP16 | | | days 3, 6, 10, 13 |
| 3 | 10 | vehicle 1// | na// | ip//ip | days 1, 4, 8, 11// |
| | | ACP16 | 232 ug/animal | | days 3, 6, 10, 13 |
| 4 | 10 | vehicle 1// | na// | ip//ip | days 1, 4, 8, 11// |
| | | ACP16 | 500 ug/animal | | days 3, 6, 10, 13 |
| 5 | 10 | anti-PD-1 RMP1-14// | 200 ug/animal// | ip//ip | days 1, 4, 8, 11// |
| | | vehicle 2 | na | | days 3, 6, 10, 13 |
| 6 | 10 | anti-PD-1 RMP1-14// | 200 ug/animal// | ip//ip | days 1, 4, 8, 11// |
| | | ACP16 | 70 ug/animal | | days 3, 6, 10, 13 |
| 7 | 10 | anti-PD-1 RMP1-14// | 200 ug/animal// | ip//ip | days 1, 4, 8, 11// |
| | | ACP16 | 232 ug/animal | | days 3, 6, 10, 13 |
| 8 | 10 | anti-PD-1 RMP1-14// | 200 ug/animal// | ip//ip | days 1, 4, 8, 11// |
| | | ACP16 | 500 ug/animal | | days 3, 6, 10, 13 |
| 9 | 10 | vehicle 1// | na//12 ug/animal | ip//ip | days 1, 4, 8, 11// |
| | | IL-2 | | | bid × 5 first day 1 dose |
| | | | | | per week × 2 |
| 10 | 10 | anti-PD-1 RMP1-14// | 200 ug/animal// | ip//ip | days 1, 4, 8, 11// |
| | | IL-2 | 12 ug/animal | | bid × 5 first day 1 dose |
| | | | | | per week × 2 |

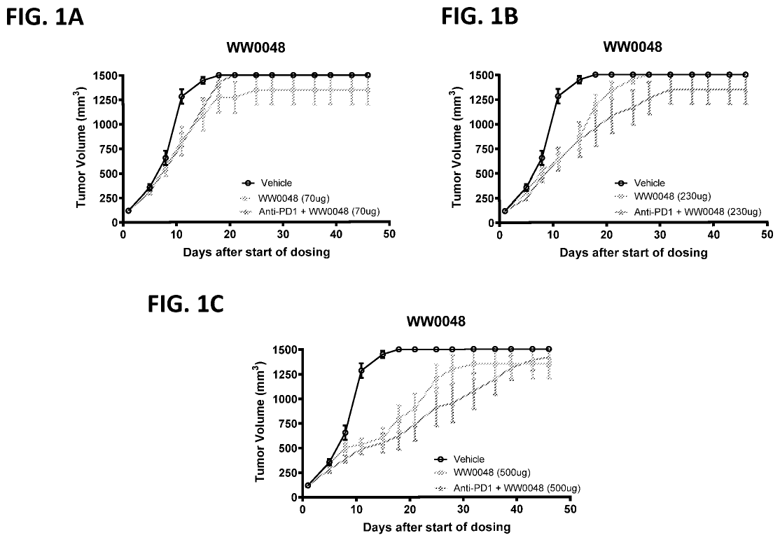

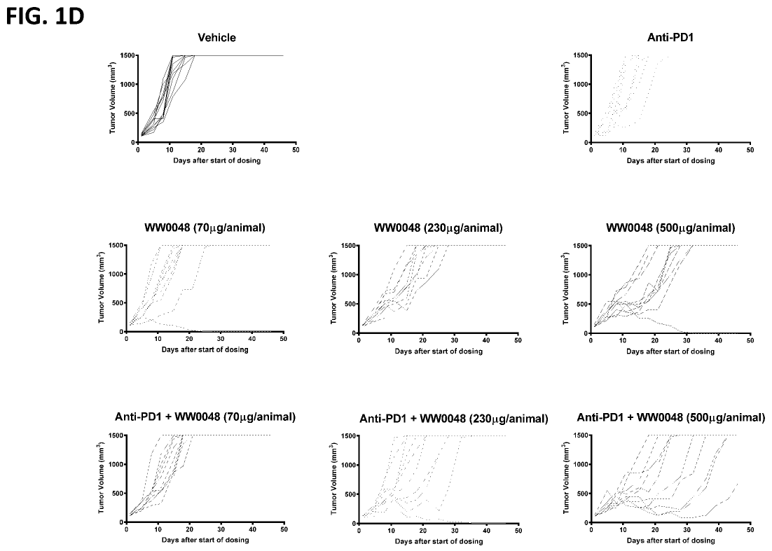

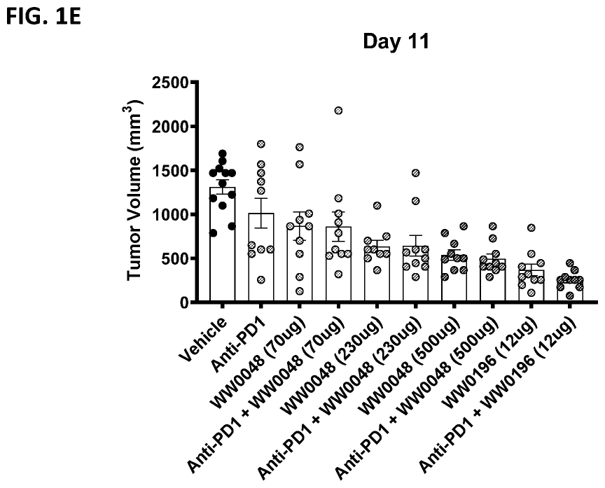

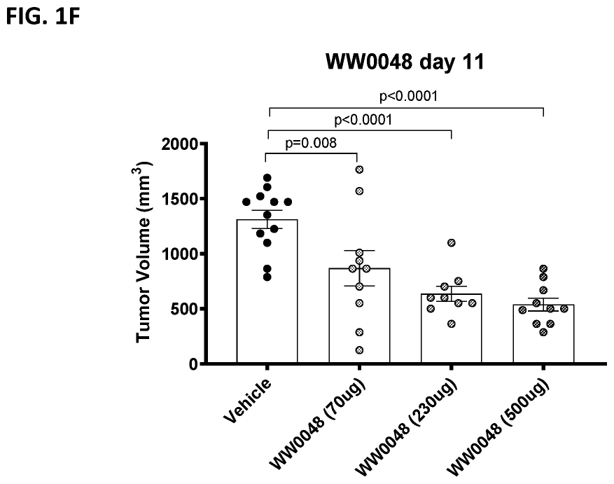

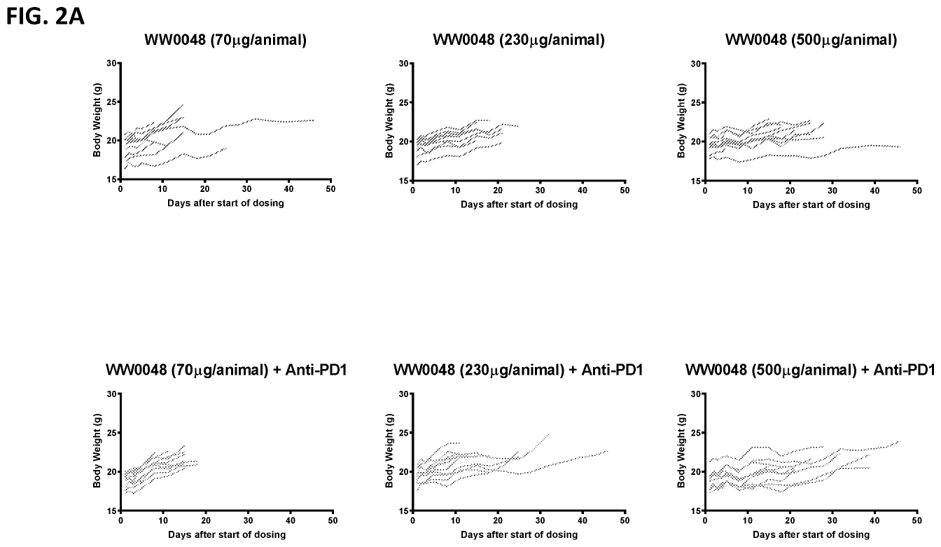

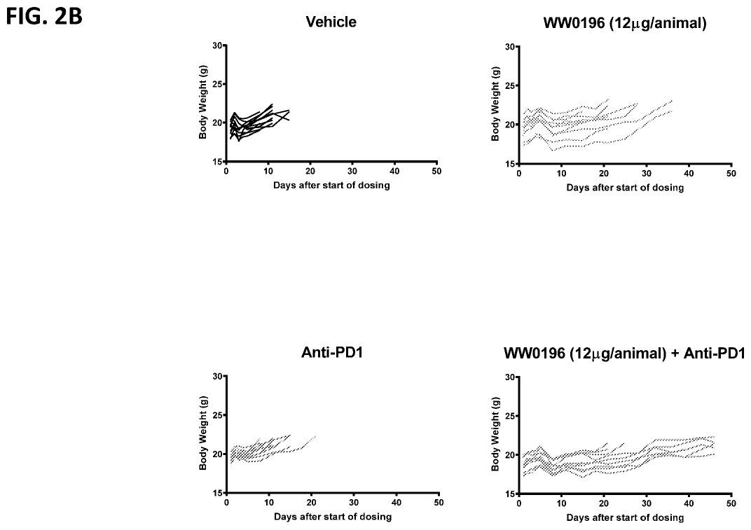

Mice were anaesthetized with isoflurane for implant of cells to reduce the ulcerations. CR female BALB/c mice were set up with 3×105 CT26 tumor cells in 000 Matrigel sc in flank. Cell Injection Volume was 0.1 mL/mouse. Mouse age at start date was 8 to 12 weeks. Pair matches were performed when tumors reach an average size of 100-150 mm³ and begin treatment. ACP16 was dosed at 70, 230 or 500 μg/animal with or without anti-PD-1 antibody (RMP1-14) at 200 μg/animal (see Table 5). Body weights were taken at initiation and then biweekly to the end. Caliper measurements were taken biweekly to the end. Any adverse reactions were to be reported immediately. Any individual animal with a single observation of >than 30% body weight loss or three consecutive measurements of >25% body weight loss was euthanized. Any group with a mean body weight loss of >20% or >10% mortality stopped dosing; the group was not euthanized and recovery is allowed. Within a group with >20% weight loss, individuals hitting the individual body weight loss endpoint were euthanized. If the group treatment related body weight loss is recovered to within 10% of the original weights, dosing resumed at a lower dose or less frequent dosing schedule. Exceptions to non-treatment body weight % recovery were allowed on a case-by-case basis. Endpoint was tumor growth delay (TGD). Animals were monitored individually. The endpoint of the experiment was a tumor volume of 1500 mm3 or 45 days, whichever comes first. Responders were followed longer. When the endpoint was reached, the animals are to be euthanized. Results are shown in FIGS. 1A-1F and FIGS. 2A-2B.

### Example 2. IL-2 in Combination with an Anti-PD1 Antibody

Inducible IL-2 prodrug was tested in a B16F10 syngeneic tumor model with or without the addition of an anti-PD1 antibody (RMP1-14). 1×10⁵ tumor cells were implanted in the flank of animals and tumor growth was monitored. Once tumors reached an average volume of 30-60 mm3, the animals were randomized and dosed as described in Table 6.

TABLE 6

| Group | N | Agent 1 | Dose | Agent 2 | Dose | Schedule |
|---|---|---|---|---|---|---|
| 1 | 10 | Vehicle | N/A | N/A | 100 μL | Biwk × 2 |
| 2 | 10 | Inducible IL-2 | 100 μg/mouse | N/A | N/A | Biwk × 2 |
| | | prodrug | | | | |
| 3 | 10 | Inducible IL-2 | 200 μg/mouse | N/A | N/A | Biwk × 2 |
| | | prodrug | | | | |
| 4 | 10 | N/A | N/A | Anti-PD-1 | 200 μg/mouse | Biwk × 2 |
| 5 | 10 | Inducible IL-2 | 100 μg/mouse | Anti-PD-1 | 200 μg/mouse | Biwk × 2 |
| | | prodrug | | | | |
| 6 | 10 | Inducible IL-2 | 200 μg/mouse | Anti-PD-1 | 200 μg/mouse | Biwk × 2 |
| | | prodrug | | | | |

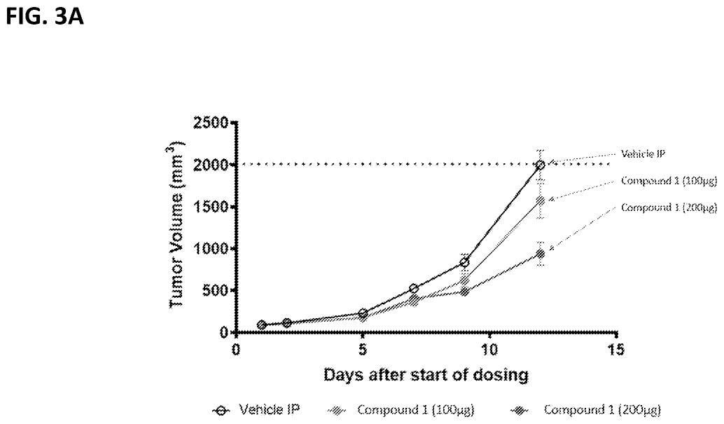

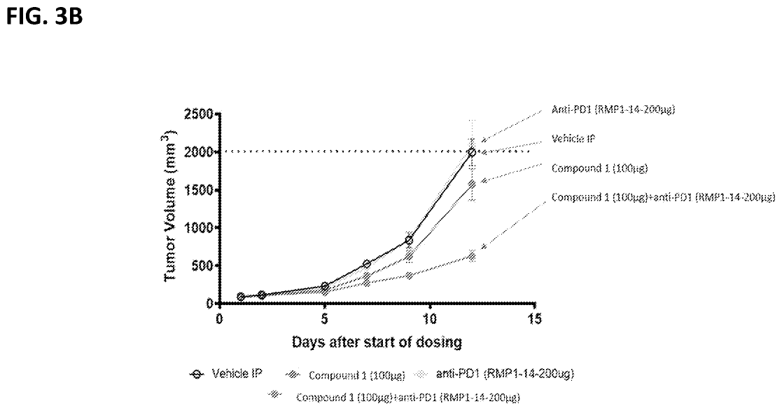

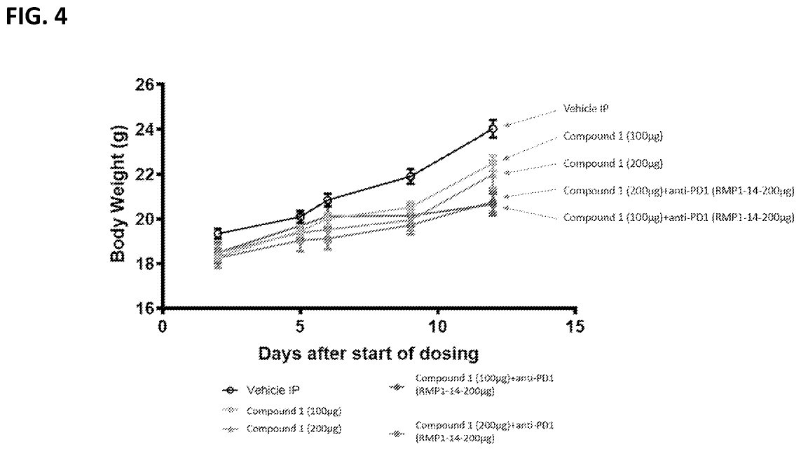

Tumor volumes and body weights were recorded three times per week with a gap of 2-3 days in between two measurements. Treatment with Inducible IL-2 prodrug showed does dependent efficacy as a monotherapy. Monotherapy with anti-PD1 in this model had no efficacy and tumor volumes in anti-PD 1 treated mice were similar to those in mice treated with vehicle only. But the combination therapy using Inducible TL-2 prodrug and anti-PD1 synergistically improved tumor control, and was more effective than either Inducible L4-2 prodrug or anti-PD-1. The results are shown in FIGS. 3A, 3B, and 4.

### Example 3. CT26 Experiments—Treatment with Inducible IFN Prodrug Alone or in Combination with Anti-PD1 Antibody

The CT26 cell line, a rapidly growing colon adenocarcinoma cell line that expresses MM4P9 in vitro, will be used. Using this tumor model, the ability of fusion proteins to affect tumor growth will be examined.

TABLE 7

| Gr. | N | Agent | Formulation dose | Route | Schedule |
|---|---|---|---|---|---|
| 1^{#} | 12 | vehicle 1// | na//na | ip//ip | days 1, 4, 8, 11// |
| | | vehicle 2 | | | days 3, 6, 10, 13 |
| 2 | 10 | vehicle 1// | na//70 ug/animal | ip//ip | days 1, 4, 8, 11// |
| | | inducible IFN prodrug | | | days 3, 6, 10, 13 |
| 3 | 10 | vehicle 1// | na// | ip//ip | days 1, 4, 8, 11// |
| | | inducible IFN prodrug | 232 ug/animal | | days 3, 6, 10, 13 |
| 4 | 10 | vehicle 1// | na// | ip//ip | days 1, 4, 8, 11// |
| | | inducible IFN prodrug | 500 ug/animal | | days 3, 6, 10, 13 |
| 5 | 10 | anti-PD-1 RMP1-14// | 200 ug/animal// | ip//ip | days 1, 4, 8, 11// |
| | | vehicle 2 | na | | days 3, 6, 10, 13 |
| 6 | 10 | anti-PD-1 RMP1-14// | 200 ug/animal// | ip//ip | days 1, 4, 8, 11// |
| | | inducible IFN prodrug | 70 ug/animal | | days 3, 6, 10, 13 |
| 7 | 10 | anti-PD-1 RMP1-14// | 200 ug/animal// | ip//ip | days 1, 4, 8, 11// |
| | | inducible IFN prodrug | 232 ug/animal | | days 3, 6, 10, 13 |
| 8 | 10 | anti-PD-1 RMP1-14// | 200 ug/animal// | ip//ip | days 1, 4, 8, 11// |
| | | ACP16 | 500 ug/animal | | days 3, 6, 10, 13 |
| 9 | 10 | vehicle 1// | na//12 ug/animal | ip//ip | days 1, 4, 8, 11// |
| | | IL-2 | | | bid × 5 first day 1 dose |
| | | | | | per week × 2 |
| 10 | 10 | anti-PD-1 RMP1-14// | 200 ug/animal// | ip//ip | days 1, 4, 8, 11// |
| | | IL-2 | 12 ug/animal | | bid × 5 first day 1 dose |
| | | | | | per week × 2 |

Mice will be anaesthetized with isoflurane for implant of cells to reduce the ulcerations. CR female BALB/c mice will be set up with 3×10⁵ CT26 tumor cells in 0% Matrigel sc in flank. Cell Injection Volume was 0.1 mL/mouse. Mouse age at start date will be 8 to 12 weeks. Pair matches will be performed when tumors reach an average size of 100-150 mm³ and begin treatment as shown in Table 7. ACP16 will be dosed at 70, 230 or 500 μg/animal with or without anti-PD-1 antibody (RMP1-14) at 200 μg/animal. Body weights will be taken at initiation and then biweekly to the end. Caliper measurements will be taken biweekly to the end. Any adverse reactions will be reported immediately. Any individual animal with a single observation of >than 30% body weight loss or three consecutive measurements of >25% body weight loss will be euthanized. Any group with a mean body weight loss of >20% or >10% mortality stopped dosing; the group will not be euthanized and recovery is allowed. Within a group with >20% weight loss, individuals hitting the individual body weight loss endpoint will be euthanized. If the group treatment related body weight loss is recovered to within 10% of the original weights, dosing will be resumed at a lower dose or less frequent dosing schedule. Exceptions to non-treatment body weight % recovery will be allowed on a case-by-case basis. Endpoint will be tumor growth delay (TGD). Animals will be monitored individually. The endpoint of the experiment will be a tumor volume of 1500 mm³ or 45 days, whichever comes first. Responders were followed longer. When the endpoint was reached, the animals will be euthanized.

### Example 4. IFN in Combination with an Anti-PD1 Antibody

Inducible IFN prodrug will be tested in a B16F10 syngeneic tumor model with or without the addition of an anti-PD1 antibody (RMP1-14). 1×10⁵ tumor cells will be implanted in the flank of animals and tumor growth will be monitored. Once tumors reached an average volume of 30-60 mm³, the animals will be randomized and dosed as described in Table 8.

TABLE 8

| Group | N | Agent 1 | Dose | Agent 2 | Dose | Schedule |
|---|---|---|---|---|---|---|
| 1 | 10 | Vehicle | N/A | N/A | 100 μL | Biwk × 2 |
| 2 | 10 | Inducible IFN | 100 μg/mouse | N/A | N/A | Biwk × 2 |
| | | prodrug | | | | |
| 3 | 10 | Inducible IFN | 200 μg/mouse | N/A | N/A | Biwk × 2 |
| | | prodrug | | | | |
| 4 | 10 | N/A | N/A | Anti-PD-1 | 200 μg/mouse | Biwk × 2 |
| 5 | 10 | Inducible IFN | 100 μg/mouse | Anti-PD-1 | 200 μg/mouse | Biwk × 2 |
| | | prodrug | | | | |
| 6 | 10 | Inducible IFN | 200 μg/mouse | Anti-PD-1 | 200 μg/mouse | Biwk × 2 |
| | | prodrug | | | | |

Tumor volumes and body weights will be recorded three times per week with a gap of 2-3 days in between two measurements. Treatment with inducible IFN prodrug will show does dependent efficacy as a monotherapy. Monotherapy with anti-PD1 in this model will have no efficacy and tumor volumes in anti-PD1 treated mice will be similar to those in mice treated with vehicle only. But, it is expected that the combination therapy using inducible IFN prodrug and anti-PD1 will synergistically improve tumor control, and will be more effective than either inducible IFN prodrug or anti-PD-1.

### Example 5. A20 Lymphoma Model

The A20 cell line, a rapidly growing B cell lymphoma cell line, was used. Using this tumor model, the ability of inducible cytokine prodrugs to affect tumor growth was examined. Since human IL-12 and IFNα2b are not cross reactive in mice, surrogate inducible cytokine prodrugs molecules were created, consisting of a mouse/human chimeric IL-12 (WW0757/636) or a mouse IFNα1 (WW0610) to explore anti-tumor responses in syngeneic hematologic cancer models.

TABLE 9

| Group | N | Agent | Dose | | Route | Schedule |
|---|---|---|---|---|---|---|
| 1 | 10 | Vehicle | — | | ip | biwk × 2 |
| 2 | 10 | WW0757/636 | 30 | ug/animal | ip | biwk × 2 |
| 3 | 10 | WW0757/636 | 100 | ug/animal | ip | biwk × 2 |
| 4 | 10 | WW0757/636 | 300 | ug/animal | ip | biwk × 2 |
| 5 | 10 | WW0610 | 45 | ug/animal | ip | biwk × 2 |
| 6 | 10 | WW0610 | 133 | ug/animal | ip | biwk × 2 |

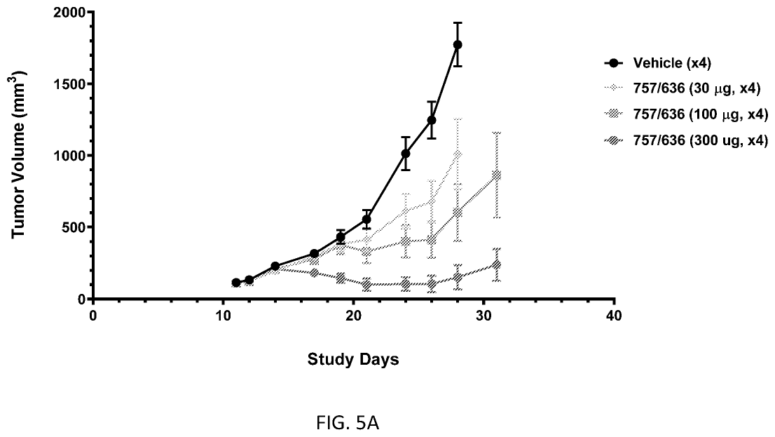

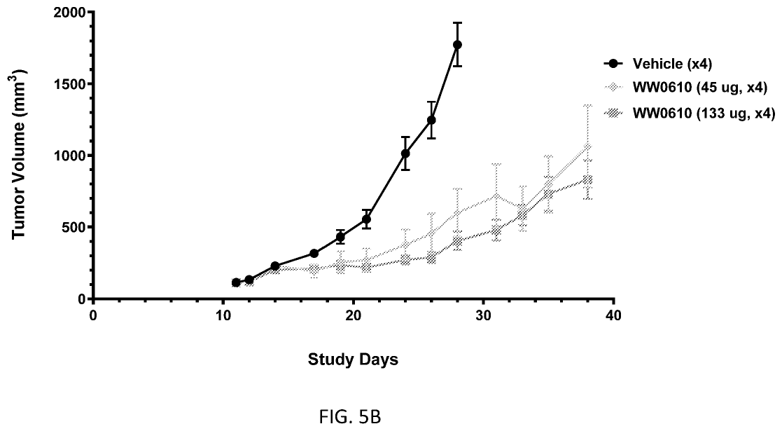

60 female BALB/c mice were set up with 5×10⁵ A20 tumor cells in 0% Matrigel sc in flank. Cell injection volume was 0.1 mL/mouse. Mouse age at start date was 8 to 12 weeks. Pair matches were performed when tumors reach an average size of 90-130 mm³ and treatment began according to Table 9. This was Day 11 of study start. Caliper measurements were taken biweekly to the end. Any adverse reactions were reported immediately. Any individual animal with a single observation of >than 25% body weight loss or three consecutive measurements of >20% body weight loss was euthanized. If any group had a mean body weight loss of >20% or >10% mortality then dosing was stopped; the group was not euthanized, and recovery was allowed. Within a group with >20% weight loss, individuals hitting the individual body weight loss endpoint were euthanized. If the group treatment related body weight loss recovered to within 10% of the original weights, dosing resumed at a lower dose or less frequent dosing schedule. Exceptions to non-treatment body weight % recovery were allowed on a case-by-case basis. Endpoint was tumor growth delay (TGD). Animals were monitored individually. The endpoint of the experiment was a tumor volume of 2000 mm³ or 40 days, whichever occurred first. When the endpoint was reached, the animals were euthanized. Results are shown in FIGS. 5A and 5B.

### Example 6. EG7.OVA Lymphoma Model

The EG7.OVA cell line, a rapidly growing T lymphoblast cell line, was used. Using this tumor model, the ability of fusion proteins to affect tumor growth was examined. Since human IL-12 and IFNα2b are not cross reactive in mice, surrogate inducible cytokine prodrugs molecules were created, consisting of a mouse/human chimeric IL-12 (WW0757/636) or a mouse IFNαl (WW0610) to explore anti-tumor responses in syngeneic hematologic cancer models.

TABLE 10

| Group | N | Agent | Dose | | Route | Schedule |
|---|---|---|---|---|---|---|
| 1 | 10 | Vehicle | — | | ip | biwk × 2 |
| 2 | 10 | WW0757/636 | 30 | ug/animal | ip | biwk × 2 |
| 3 | 10 | WW0757/636 | 100 | ug/animal | ip | biwk × 2 |
| 4 | 10 | WW0757/636 | 300 | ug/animal | ip | biwk × 2 |
| 5 | 10 | WW0610 | 45 | ug/animal | ip | biwk × 2 |
| 6 | 10 | WW0610 | 133 | ug/animal | ip | biwk × 2 |

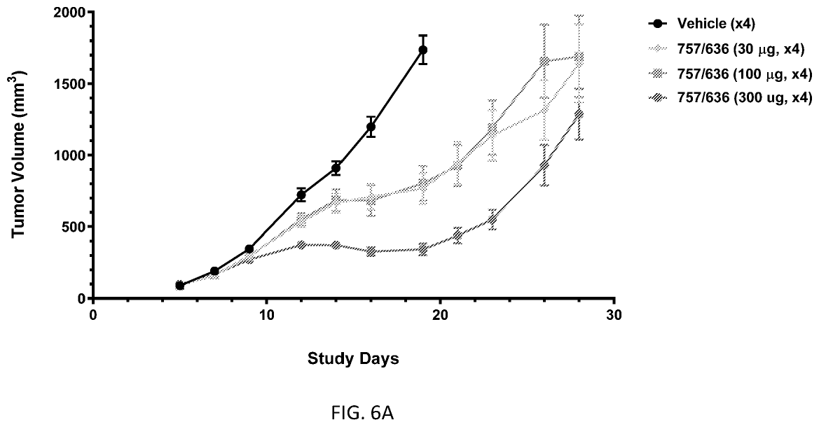

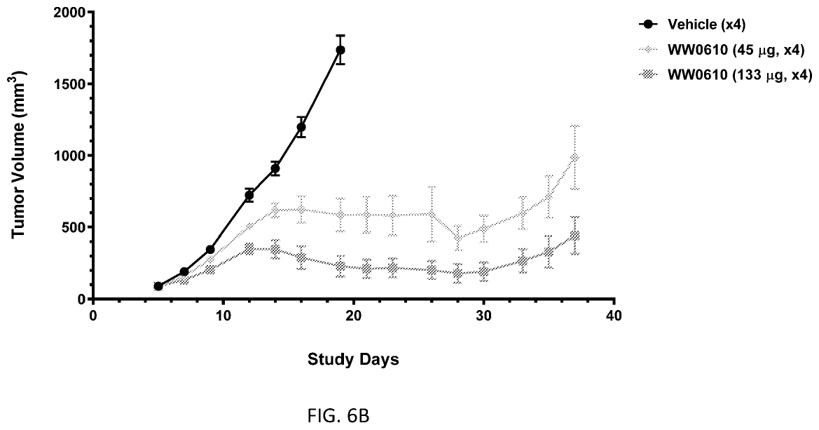

60 female C57Bl/6 mice were set up with 10×105 EG7. OVA tumor cells in 0% Matrigel sc in flank. Cell injection volume was 0.1 mL/mouse. Mouse age at start date was 8 to 12 weeks. Pair matches were performed when tumors reach an average size of 63-135 mm³ and begin treatment according to Table 10. This was Day 5 of study start. Caliper measurements were taken biweekly to the end. Any adverse reactions were reported immediately. Any individual animal with a single observation of >than 25% body weight loss or three consecutive measurements of >20% body weight loss was euthanized. If any group had a mean body weight loss of >20% or >10% mortality then dosing was stopped; the group was not euthanized, and recovery was allowed. Within a group with >20% weight loss, individuals hitting the individual body weight loss endpoint were euthanized. If the group treatment related body weight loss was recovered to within 10% of the original weights, dosing resumed at a lower dose or less frequent dosing schedule. Exceptions to non-treatment body weight % recovery were allowed on a case-by-case basis. Endpoint was tumor growth delay (TGD). Animals were monitored individually. The endpoint of the experiment was a tumor volume of 2000 mm³ or 40 days, whichever occurred first. When the endpoint was reached, the animals were euthanized. Results are shown in FIGS. 6A and 6B.

### Example 7. CT26 Experiments—Treatment with Inducible IL-2 Prodrug or Anti-PD1 Alone or in Combination

The CT26 cell line, a rapidly growing colon adenocarcinoma cell line that expresses MMP9 in vitro, was used. Using this tumor model, the ability of fusion proteins to affect tumor growth was examined.

TABLE 11

| | | | Inducible IL- | | | |
|---|---|---|---|---|---|---|
| | | | 2 prodrug | Anti-PD1 | Inducible IL-2 | Anti-PD1 |
| Gr. | N | Treatment | dose | Dose | prodrug schedule | schedule |
| 1^{#} | 8 | vehicle | na | na | na | na |
| 2 | 8 | inducible IL-2 | 100 μg/animal | na | Days 1 and 8 | na |
| | | prodrug | | | | |
| 3 | 8 | Anti-PD-1 | na | 200 μg/animal | na | Days 1, 4, 8 and 11 |
| 4 | 8 | inducible IL-2 | 100 μg/animal | 200 μg/animal | Days 1 and 8 | Days 1, 4, 8 and 11 |
| | | prodrug + | | | | |
| | | anti-PD-1 | | | | |

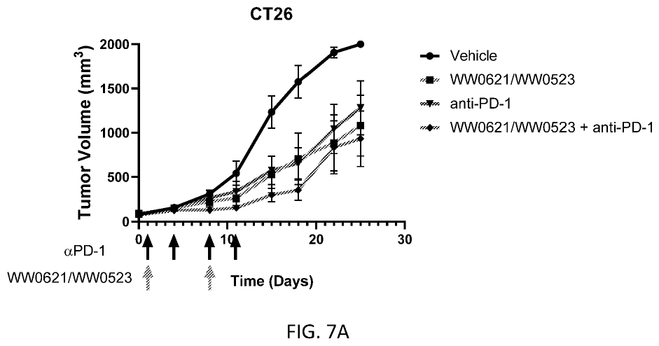

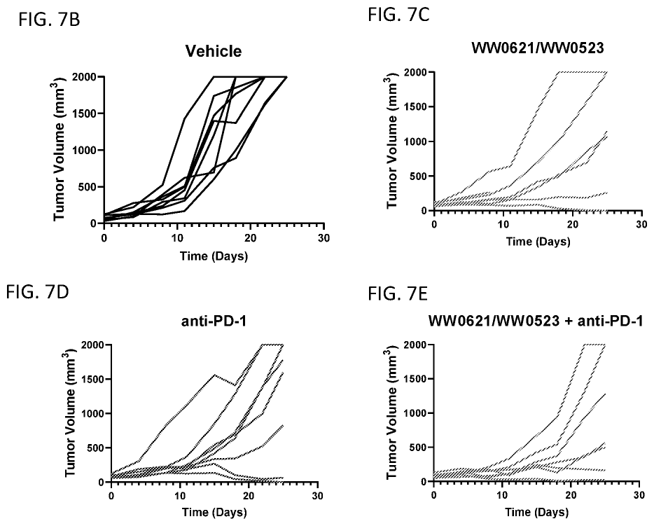

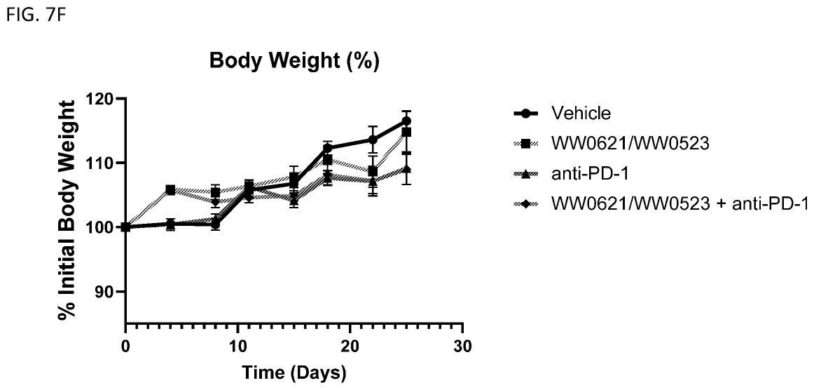

Mice were anaesthetized with isoflurane for implant of cells to reduce the ulcerations. Female BALB/c mice were set up with 1.5×10⁵ CT26 tumor cells sc in flank. Cell Injection Volume was 0.1 mL/mouse. Mouse age at start date was 8 to 12 weeks. Pair matches were performed when tumors reached an average size of 100-150 mm³ and treatment began according to Table 11. The inducible IL-2 prodrug was a two-chain polypeptide that contained WW0621 and WW0523, and the anti-PD-1 antibody was RMP1-14. Body weights were taken at initiation and then biweekly to the end of the study. Caliper measurements of tumor size were taken biweekly to the end of the study. Any adverse reactions were reported immediately. Any individual animal with a single observation of >than 30% body weight loss or three consecutive measurements of >25% body weight loss was euthanized. Any group with a mean body weight loss of >20% or >1000 mortality resulted in stopped dosing; the group was not euthanized, and recovery was allowed. Animals were monitored individually. The endpoint of the study was a tumor volume of 1500 mm³ or 45 days, whichever comes first. Responders were followed longer. When the endpoint was reached, the animals were euthanized. Results are shown in FIGS. 7A-7F.

### 8. CONSTRUCTS

The elements of the polypeptide constructs provided in Table 8 contain the abbreviations as follows: “X” refers to a linker. “X” refers to a cleavable linker. Linker 3 refers to a linker that comprises a CTSL-1 substrate motif sequence.

TABLE 11
Exemplary inducible cytokine prodrug constructs

| Construct # | Construct Description |
|---|---|
| WW0706 | anti-HSA-X-human_p40-L-mouse_p35-XL- |
| | Fab_Lanbda_Blocker_(Blocker = |
| | Lanbda_Fab_R27E_T32D_IGLC2-01_X = Linker2) |
| WW0707 | anti-HSA-X-human_p40-L-human_p35-XL- |
| | Fab_Lambda_Blocker_(Blocker = |
| | Lambda_Fab_R27E_T32D_IGLC2_01_X = Linker2) |
| WW0708 | anti-HSA-X-human_p40-L-mouse_p35-XL- |
| | Fab_Lambda_Blocker_(Blocker = |
| | Lambda_Fab_S30E_IGLC2-01_X = Linker2) |
| WW0709 | anti-HSA-X-human_p40-L-human_p35-XL- |
| | Fab_Lambda_Blocker_(Blocker = |
| | Lambda_Fab_S30E_IGLC2-01_X = Linker2) |
| WW0710 | anti-HSA-X-human_p40-L-mouse_p35-XL- |
| | Fab_Lambda_Blocker_(Blocker = |
| | Lambda_Fab_S30E_N31E_IGLC2-01_X = Linker2) |
| WW0711 | anti-HSA-X-human_p40-L-human_p35-XL- |
| | Fab_Lambda_Blocker_(Blocker = |
| | Lambda_Fab_S30E_N31E_IGLC2-01_X = Linker2) |
| WW0700 | anti-HSA-X-human_p40-L-mouse_p35-XL- |
| | Fab_Lambda_Blocker_(Blocker = |
| | Lambda_Fab_N31W_IGLC2-01_X = Linker2) |
| WW0701 | anti-HSA-X-human_p40-L-human_p35-XL- |
| | Fab_Lambda_Blocker_(Blocker = |
| | Lambda_Fab_N31E_IGLC2-01_X = Linker2) |
| WW0712 | anti-HSA-X-human_p40-L-mouse_p35-XL- |
| | Fab_Lambda_Blocker_(Blocker = |
| | Lambda_Fab_IGLC2-01_X = Linker3) |
| WW0713 | anti-HSA-X-human_p40-L-human_p35-XL- |
| | Fab_Lambda_Blocker_(Blocker = |
| | Lambda_Fab_IGLC2-01_X = Linker3) |
| WW0714 | anti-HSA-X-human_p40-L-mouse_p35-XL- |
| | Fab_Lambda_Blocker_(Blocker = |
| | LAmbda_Fab_N31E_IGLC2-01_X = Linker3) |
| WW0715 | anti-HSA-X-human_p40-L-human_p35-XL- |
| | Fab_Lambda_Blocker_(Blocker = |
| | LAmbda_Fab_N31E_IGLC2-01_X = Linker3) |
| WW0805 | human_p35-X-anti-HSA-L-Blocker_(Blocker = |
| | Opt1_Hv_D53E_D61E_V1-Vh_X = Linker3) |
| WW0754 | anti-HSA-X-Human_p35-XL- |
| | Fab_Lambda_Blocker_(Blocker = |
| | Lambda_Fab_IGLC2-01_X = Linker2) |
| WW0756 | anti-HSA-X-Human_p35-XL- |
| | Fab_Lambda_Blocker_(Blocker = |
| | Lambda_Fab_S30D_N31E_IGLC2-01_X = Linker2) |
| WW0762 | anti-HSA-X-Human_p35-XL- |
| | Fab_Lambda_Blocker_(Blocker = |
| | Lambda_Fab_IGLC2-01_X = Linker3) |
| WW0770 | human_p40-L-human_p35-X-anti-HSA-L- |
| | Fab_Lambda_Blocker_(Blocker = |
| | Lambda_Fab_IGLC2-01_X = Linker2) |
| WW0636 | Human_IL12B_(p40) |
| WW0727 | Fab_Heavy_Blocker_(Blocker = |
| | IL-12_Heavy_Fab_D53E_D61E_IgG1_Fab) |
| WW0045 | Blocker2-Linker-(cleav. link.)-IL2-(cleav. link.)- |
| | (anti-HSA)-6xHis |
| WW0046 | (anti-HSA)-(cleav. link.)-Blocker2-Linker- |
| | (cleav. link.)-IL2-6xHis |
| WW0203 | IL2-(cleav. link.)-(anti-HSA)-linker |
| | (cleav. link.)-blocker2 |
| WW0204 | IL2-X-anti-HSA-LX-blocker_(X = |
| | Linker4_Blocker = Vh-V1) |
| WW0205 | IL2-X-anti-HSA-LX-blocker_(X = |
| | Linker5_Blocker = Vh-V1) |
| WW0234 | IL2-X-HSA-LX-blocker_(X = |
| | Linker 1; Blocker = 3TOW69) |
| WW0235 | IL2-X-HSA-LX-blocker_(X = |
| | Linker 1; Blocker = 3TOW85) |
| WW0236 | IL2-X-HSA-LX-blocker_(X = |
| | Linker 1; Blocker = 2TOW91) |
| WW0308 | IL2-X-HSA-LX-blocker(QAPRL_FR2)_(X = |
| | Linker 1; Blocker = Vh/V1 |
| WW0415 | IL2-X-anti-HSA-LX-blocker_(Blocker = |
| | VHVL-F2.high. A02_Vh/V1_A46S; X = Linker 2) |
| WW0621 | IL2-X-anti-HSA-LX-Heavy_blocker_Fab_ |
| | (Blocker = VH-CH1; X = Linker 3) |
| WW0520 | IL2-X-anti-HSA-LX-Heavy_blocker_Fab_ |
| | (Blocker = VH-CH1; X = Linker 2) |
| WW0735 | IL2-X-anti-HSA-LL-Heavy_blocker_Fab_ |
| | (Blocker = VH-CH1_X = Linker2) |
| WW0736 | IL2-X-anti-HSA-LL-Heavy_Blocker_Fab_ |
| | (Blocker = VH-CH1_X = Linker3) |

9. SEQUENCE DISCLOSURE

| SEQ | | |
|---|---|---|
| ID | | |
| NO: | Description | Sequence |
| 1 | WW0621 | APTSSSTKKTQLQLEHLLLDLQMILNGINN |
| | (Heterodimeric | YKNPKLTRMLTFKFYMPKKATELKHLQCLE |
| | inducible IL-2 | EELKPLEEVLNLAQSKNFHLRPRDLISNIN |
| | prodrug) | VIVLELKGSETTFMCEYADETATIVEFLNR |
| | | WITFCQSIISTLTSGGPALFKSSFPPGSEV |
| | | QLVESGGGLVQPGNSLRLSCAASGFTFSKF |
| | | GMSWVRQAPGKGLEWVSSISGSGRDTLYAE |
| | | SVKGRFTISRDNAKTTLYLQMNSLRPEDTA |
| | | VYYCTIGGSLSVSSQGTLVTVSSGGGGSGG |
| | | GGSGGGGSGGGGSGGGGSGGGGSSGGPALF |
| | | KSSFPPGSEVQLVESGGGLVQPGGSLRLSC |
| | | AASGFTFSSYTLAWVRQAPGKGLEWVAAID |
| | | SSSYTYSPDTVRGRFTISRDNAKNSLYLQM |
| | | NSLRAEDTAVYYCARDSNWDALDYWGQGTT |
| | | VTVSSASTKGPSVFPLAPSSKSTSGGTAAL |
| | | GCLVKDYFPEPVTVSWNSGALTSGVHTFPA |
| | | VLQSSGLYSLSSVVTVPSSSLGTQTYICNV |
| | | NHKPSNTKVDKRVEPKSC** |
| 2 | WW0520 | APTSSSTKKTQLQLEHLLLDLQMILNGINN |
| | (Heterodimeric | YKNPKLTRMLTFKFYMPKKATELKHLQCLE |
| | inducible IL-2 | EELKPLEEVLNLAQSKNFHLRPRDLISNIN |
| | prodrug) | VIVLELKGSETTFMCEYADETATIVEFLNR |
| | | WITFCQSIISTLTSGGPGPAGLYAQPGSEV |
| | | QLVESGGGLVQPGNSLRLSCAASGFTFSKF |
| | | GMSWVRQAPGKGLEWVSSISGSGRDTLYAE |
| | | SVKGRFTISRDNAKTTLYLQMNSLRPEDTA |
| | | VYYCTIGGSLSVSSQGTLVTVSSGGGGSGG |
| | | GGSGGGGSGGGGSGGGGSGGGGSSGGPGPA |
| | | GLYAQPGSEVQLVESGGGLVQPGGSLRLSC |
| | | AASGFTFSSYTLAWVRQAPGKGLEWVAAID |
| | | SSSYTYSPDTVRGRFTISRDNAKNSLYLQM |
| | | NSLRAEDTAVYYCARDSNWDALDYWGQGTT |
| | | VTVSSASTKGPSVFPLAPSSKSTSGGTAAL |
| | | GCLVKDYFPEPVTVSWNSGALTSGVHTFPA |
| | | VLQSSGLYSLSSVVTVPSSSLGTQTYICNV |
| | | NHKPSNTKVDKRVEPKSC** |
| 3 | WW0735 | APTSSSTKKTQLQLEHLLLDLQMILNGINN |
| | (Heterodimeric | YKNPKLTRMLTFKFYMPKKATELKHLQCLE |
| | inducible IL-2 | EELKPLEEVLNLAQSKNFHLRPRDLISNIN |
| | prodrug) | VIVLELKGSETTFMCEYADETATIVEFLNR |
| | | WITFCQSIISTLTSGGPGPAGLYAQPGSEV |
| | | QLVESGGGLVQPGNSLRLSCAASGFTFSKF |
| | | GMSWVRQAPGKGLEWVSSISGSGRDTLYAE |
| | | SVKGRFTISRDNAKTTLYLQMNSLRPEDTA |
| | | VYYCTIGGSLSVSSQGTLVTVSSGGGGSGG |
| | | GGSGGGGSGGGGSGGGGSGGGGSGGGGSGG |
| | | GGSGGGGSEVQLVESGGGLVQPGGSLRLSC |
| | | AASGFTFSSYTLAWVRQAPGKGLEWVAAID |
| | | SSSYTYSPDTVRGRFTISRDNAKNSLYLQM |
| | | NSLRAEDTAVYYCARDSNWDALDYWGQGTT |
| | | VTVSSASTKGPSVFPLAPSSKSTSGGTAAL |
| | | GCLVKDYFPEPVTVSWNSGALTSGVHTFPA |
| | | VLQSSGLYSLSSVVTVPSSSLGTQTYICNV |
| | | NHKPSNTKVDKRVEPKSC |
| 4 | WW0736 | APTSSSTKKTQLQLEHLLLDLQMILNGINN |
| | (Heterodimeric | YKNPKLTRMLTFKFYMPKKATELKHLQCLE |
| | inducible IL-2 | EELKPLEEVLNLAQSKNFHLRPRDLISNIN |
| | prodrug) | VIVLELKGSETTFMCEYADETATIVEFLNR |
| | | WITFCQSIISTLTSGGPALFKSSFPPGSEV |
| | | QLVESGGGLVQPGNSLRLSCAASGFTFSKF |
| | | GMSWVRQAPGKGLEWVSSISGSGRDTLYAE |
| | | SVKGRFTISRDNAKTTLYLQMNSLRPEDTA |
| | | VYYCTIGGSLSVSSQGTLVTVSSGGGGSGG |
| | | GGSGGGGSGGGGSGGGGSGGGGSGGGGSGG |
| | | GGSGGGGSEVQLVESGGGLVQPGGSLRLSC |
| | | AASGFTFSSYTLAWVRQAPGKGLEWVAAID |
| | | SSSYTYSPDTVRGRFTISRDNAKNSLYLQM |
| | | NSLRAEDTAVYYCARDSNWDALDYWGQGTT |
| | | VTVSSASTKGPSVFPLAPSSKSTSGGTAAL |
| | | GCLVKDYFPEPVTVSWNSGALTSGVHTFPA |
| | | VLQSSGLYSLSSVVTVPSSSLGTQTYICNV |
| | | NHKPSNTKVDKRVEPKSC |
| 5 | WW0523 | DIQMTQSPSSLSASVGDRVTITCKAREKLW |
| | Kappa_blocker_Fab | SAVAWYQQKPGKAPKSLIYSASFRYSGVPS |
| | _(Blocker=VHVL.F | RFSGSGSGTDFTLTISSLQPEDFATYYCQQ |
| | 2.high. | YYTYPYTFGGGTKVEIKRTVAAPSVFIFPP |
| | A02_A46S_Kappa) | SDEQLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |
| 6 | WW0758 | EVQLVESGGGLVQPGNSLRLSCAASGFTFS |
| | Heterodimeric IL- | KFGMSWVRQAPGKGLEWVSSISGSGRDTLY |
| | 12 polypeptide, | AESVKGRFTISRDNAKTTLYLQMNSLRPED |
| | anti-HSA sdAb, | TAVYYCTIGGSLSVSSQGTLVTVSSsggpA |
| | scFv Blocker, 2 | LFKSSFPpgsrnlpvatpdpgmfpclhhsq |
| | cleavage sites | nllravsnmlqkarqtlefypctseeidhe |
| | | ditkdktstveaclpleltknesclnsret |
| | | sfitngsclasrktsfmmalclssiyedlk |
| | | myqvefktmnakllmdpkrqifldqnmlav |
| | | idelmqalnfnsetvpqkssleepdfyktk |
| | | iklcillhafriravtidrvmsylnassgg |
| | | pALFKSSFPpgsggggsggggsggggsggg |
| | | gsggggsggggsQSVLTQPPSVSGAPGQRV |
| | | TISCSGSRSNIGSNTVKWYQQLPGTAPKLL |
| | | IYYNDQRPSGVPDRFSGSKSGTSASLAITG |
| | | LQAEDEADYYCQSYDRYTHPALLFGTGTKV |
| | | TVLggggsggggsggggsQVQLVESGGGVV |
| | | QPGRSLRLSCAASGFTFSSYGMHWVRQAPG |
| | | KGLEWVAFIRYeGSNKYYAeSVKGRFTISR |
| | | DNSKNTLYLQMNSLRAEDTAVYYCKTHGSH |
| | | DNWGQGTMVTVSS** |
| 7 | WW0805 | rnlpvatpdpgmfpclhhsqnllravsnml |
| | Heterodimeric IL- | qkarqtlefypctseeidheditkdktstv |
| | 12 polypeptide, | eaclpleltknesclnsretsfitngscla |
| | anti-HSA sdAb, | srktsfmmalclssiyedlkmyqvefktmn |
| | scFv Blocker, 1 | akllmdpkrqifldqnmlavidelmqalnf |
| | cleavage site | nsetvpqkssleepdfyktkiklcillhaf |
| | | riravtidrvmsylnassggpALFKSSFPp |
| | | gsEVQLVESGGGLVQPGNSLRLSCAASGFT |
| | | FSKFGMSWVRQAPGKGLEWVSSISGSGRDT |
| | | LYAESVKGRFTISRDNAKTTLYLQMNSLRP |
| | | EDTAVYYCTIGGSLSVSSQGTLVTVSSggg |
| | | gsggggsggggsggggsggggsggggsQSV |
| | | LTQPPSVSGAPGQRVTISCSGSRSNIGSNT |
| | | VKWYQQLPGTAPKLLIYYNDQRPSGVPDRF |
| | | SGSKSGTSASLAITGLQAEDEADYYCQSYD |
| | | RYTHPALLFGTGTKVTVLggggsggggsgg |
| | | ggsQVQLVESGGGVVQPGRSLRLSCAASGF |
| | | TFSSYGMHWVRQAPGKGLEWVAFIRYeGSN |
| | | KYYAeSVKGRFTISRDNSKNTLYLQMNSLR |
| | | AEDTAVYYCKTHGSHDNWGQGTMVTVSS** |
| 8 | WW0754 | EVQLVESGGGLVQPGNSLRLSCAASGFTFS |
| | Heterodimeric IL- | KFGMSWVRQAPGKGLEWVSSISGSGRDTLY |
| | 12 polypeptide, | AESVKGRFTISRDNAKTTLYLQMNSLRPED |
| | anti-HSA sdAb, | TAVYYCTIGGSLSVSSQGTLVTVSSsggpG |
| | Fab Blocker, 2 | PAGLYAQpgsrnlpvatpdpgmfpclhhsq |
| | cleavage sites | nllravsnmlqkarqtlefypctseeidhe |
| | | ditkdktstveaclpleltknesclnsret |
| | | sfitngsclasrktsfmmalclssiyedlk |
| | | myqvefktmnakllmdpkrqifldqnmlav |
| | | idelmqalnfnsetvpqkssleepdfyktk |
| | | iklcillhafriravtidrvmsylnassgg |
| | | pGPAGLYAQpgsggggggggsggggsgggg |
| | | sggggsggggsQSVLTQPPSVSGAPGQRVT |
| | | ISCSGSRSNIGSNTVKWYQQLPGTAPKLLI |
| | | YYNDQRPSGVPDRFSGSKSGTSASLAITGL |
| | | QAEDEADYYCQSYDRYTHPALLFGTGTKVT |
| | | VLgqpkaapsvtlfppsseelqankatlvc |
| | | lisdfypgavtvawkadsspvkagvetttp |
| | | skqsnnkyaassylsltpeqwkshrsyscq |
| | | vthegstvektvaptecs** |
| 9 | WW0756 | EVQLVESGGGLVQPGNSLRLSCAASGFTFS |
| | Heterodimeric IL- | KFGMSWVRQAPGKGLEWVSSISGSGRDTLY |
| | 12 polypeptide, | AESVKGRFTISRDNAKTTLYLQMNSLRPED |
| | anti-HSA sdAb, | TAVYYCTIGGSLSVSSQGTLVTVSSsggpG |
| | Fab Blocker, 2 | PAGLYAQpgsrnlpvatpdpgmfpclhhsq |
| | cleavage sites | nllravsnmlqkarqtlefypctseeidhe |
| | | ditkdktstveaclpleltknesclnsret |
| | | sfitngsclasrktsfmmalclssiyedlk |
| | | myqvefktmnakllmdpkrqifldqnmlav |
| | | idelmqalnfnsetvpqkssleepdfyktk |
| | | iklcillhafriravtidrvmsylnassgg |
| | | pGPAGLYAQpgsggggggggsggggsgggg |
| | | sggggggggsQSVLTQPPSVSGAPGQRVTI |
| | | SCSGSRSNIGdeTVKWYQQLPGTAPKLLIY |
| | | YNDQRPSGVPDRFSGSKSGTSASLAITGLQ |
| | | AEDEADYYCQSYDRYTHPALLFGTGTKVTV |
| | | Lgqpkaapsvtlfppsseelqankatlvcl |
| | | isdfypgavtvawkadsspvkagvetttps |
| | | kqsnnkyaassylsltpeqwkshrsyscqv |
| | | thegstvektvaptecs** |
| 10 | WW0762 | EVQLVESGGGLVQPGNSLRLSCAASGFTFS |
| | Heterodimeric IL- | KFGMSWVRQAPGKGLEWVSSISGSGRDTLY |
| | 12 polypeptide, | AESVKGRFTISRDNAKTTLYLQMNSLRPED |
| | anti-HSA sdAb, | TAVYYCTIGGSLSVSSQGTLVTVSSsggpA |
| | Fab Blocker, 2 | LFKSSFPpgsrnlpvatpdpgmfpclhhsq |
| | cleavage sites | nllravsnmlqkarqtlefypctseeidhe |
| | | ditkdktstveaclpleltknesclnsret |
| | | sfitngsclasrktsfmmalclssiyedlk |
| | | myqvefktmnakllmdpkrqifldqnmlav |
| | | idelmqalnfnsetvpqkssleepdfyktk |
| | | iklcillhafriravtidrvmsylnassgg |
| | | pALFKSSFPpgsggggggggggggsggggs |
| | | ggggsggggsQSVLTQPPSVSGAPGQRVTI |
| | | SCSGSRSNIGSNTVKWYQQLPGTAPKLLIY |
| | | YNDQRPSGVPDRFSGSKSGTSASLAITGLQ |
| | | AEDEADYYCQSYDRYTHPALLFGTGTKVTV |
| | | Lgqpkaapsvtlfppsseelqankatlvcl |
| | | isdfypgavtvawkadsspvkagvetttps |
| | | kqsnnkyaassylsltpeqwkshrsyscqv |
| | | thegstvektvaptecs** |
| 11 | WW0770 | iwelkkdvyvveldwypdapgemvvltcdt |
| | Monomeric IL-12 | peedgitwtldqssevlgsgktltiqvkef |
| | polypeptide, anti- | gdagqytchkggevlshsllllhkkedgiw |
| | HSA sdAb, Fab | stdilkdqkepknktflrceaknysgrftc |
| | Blocker, 1 | wwlttistdltfsvkssrgssdpqgvtcga |
| | cleavage site | atlsaervrgdnkeyeysvecqedsacpaa |
| | | eeslpievmvdavhklkyenytssffirdi |
| | | ikpdppknlqlkplknsrqvevsweypdtw |
| | | stphsyfsltfcvqvqgkskrekkdrvftd |
| | | ktsatvicrknasisvraqdryyssswsew |
| | | asvpcsggggsggggsggggsrnlpvatpd |
| | | pgmfpclhhsqnllravsnmlqkarqtlef |
| | | ypctseeidheditkdktstveaclplelt |
| | | knesclnsretsfitngsclasrktsfmma |
| | | lclssiyedlkmyqvefktmnakllmdpkr |
| | | qifldqnmlavidelmqalnfnsetvpqks |
| | | sleepdfyktkiklcillhafriravtidr |
| | | vmsylnassggpGPAGLYAQpgsEVQLVES |
| | | GGGLVQPGNSLRLSCAASGFTFSKFGMSWV |
| | | RQAPGKGLEWVSSISGSGRDTLYAESVKGR |
| | | FTISRDNAKTTLYLQMNSLRPEDTAVYYCT |
| | | IGGSLSVSSQGTLVTVSSggggsggggsgg |
| | | ggsggggsggggsggggsQSVLTQPPSVSG |
| | | APGQRVTISCSGSRSNIGSNTVKWYQQLPG |
| | | TAPKLLIYYNDQRPSGVPDRFSGSKSGTSA |
| | | SLAITGLQAEDEADYYCQSYDRYTHPALLF |
| | | GTGTKVTVLgqpkaapsvtlfppsseelqa |
| | | nkatlvclisdfypgavtvawkadsspvka |
| | | gvetttpskqsnnkyaassylsltpeqwks |
| | | hrsyscqvthegstvektvaptecs** |
| 12 | WW0636 | iwelkkdvyvveldwypdapgemvvltcdt |
| | | peedgitwtldqssevlgsgktltiqvkef |
| | | gdagqytchkggevlshsllllhkkedgiw |
| | | stdilkdqkepknktflrceaknysgrftc |
| | | wwlttistdltfsvkssrgssdpqgvtcga |
| | | atlsaervrgdnkeyeysvecqedsacpaa |
| | | eeslpievmvdavhklkyenytssffirdi |
| | | ikpdppknlqlkplknsrqvevsweypdtw |
| | | stphsyfsltfcvqvqgkskrekkdrvftd |
| | | ktsatvicrknasisvraqdryyssswsew |
| | | asvpCS** |
| 13 | WW0727 | QVQLVESGGGVVQPGRSLRLSCAASGFTFS |
| | Monomeric IL-12 | SYGMHWVRQAPGKGLEWVAFIRYeGSNKYY |
| | polypeptide, anti- | AeSVKGRFTISRDNSKNTLYLQMNSLRAED |
| | HSA sdAb, Fab | TAVYYCKTHGSHDNWGQGTMVTVSSastkg |
| | Blocker, 2 | psvfplapsskstsggtaalgclvkdyfpe |
| | cleavage sites | pvtvswnsgaltsgvhtfpavlqssglysl |
| | | ssvvtvpssslgtqtyicnvnhkpsntkvd |
| | | krvepksc** |
| 14 | WW0613 | EVQLVESGGGLVQPGNSLRLSCAASGFTFS |
| | | KFGMSWVRQAPGKGLEWVSSISGSGRDTLY |
| | | AESVKGRFTISRDNAKTTLYLQMNSLRPED |
| | | TAVYYCTIGGSLSVSSQGTLVTVSSSGGPA |
| | | LFKSSFPPGSCDLPQTHSLGSRRTLMLLAQ |
| | | MRRISLFSCLKDRHDFGFPQEEFGNQFQKA |
| | | ETIPVLHEMIQQIFNLFSTKDSSAAWDETL |
| | | LDKFYTELYQQLNDLEACVIQGVGVTETPL |
| | | MKEDSILAVRKYFQRITLYLKEKKYSPCAW |
| | | EVVRAEIMRSFSLSTNLQESLRSKESGGPA |
| | | LFKSSFPPGSEVQLVESGGGLVQPGNSLRL |
| | | SCAASGFTFSKFGMSWVRQAPGKGLEWVSS |
| | | ISGSGRDTLYAESVKGRFTISRDNAKTTLY |
| | | LQMNSLRPEDTAVYYCTIGGSLSVSSQGTL |
| | | VTVSS |
| 15 | WW0614 | EVQLVESGGGLVQPGNSLRLSCAASGFTFS |
| | | KFGMSWVRQAPGKGLEWVSSISGSGRDTLY |
| | | AESVKGRFTISRDNAKTTLYLQMNSLRPED |
| | | TAVYYCTIGGSLSVSSQGTLVTVSSSGGPP |
| | | LAQKLKSSPGSCDLPQTHSLGSRRTLMLLA |
| | | QMRRISLFSCLKDRHDFGFPQEEFGNQFQK |
| | | AETIPVLHEMIQQIFNLFSTKDSSAAWDET |
| | | LLDKFYTELYQQLNDLEACVIQGVGVTETP |
| | | LMKEDSILAVRKYFQRITLYLKEKKYSPCA |
| | | WEVVRAEIMRSFSLSTNLQESLRSKESGGP |
| | | PLAQKLKSSPGSEVQLVESGGGLVQPGNSL |
| | | RLSCAASGFTFSKFGMSWVRQAPGKGLEWV |
| | | SSISGSGRDTLYAESVKGRFTISRDNAKTT |
| | | LYLQMNSLRPEDTAVYYCTIGGSLSVSSQG |
| | | TLVTVSS |
| 16 | WW0819 | EAHKSEIAHRYNDLGEQHFKGLVLIAFSQY |
| | | LQKCSYDEHAKLVQEVTDFAKTCVADESAA |
| | | NCDKSLHTLFGDKLCAIPNLRENYGELADC |
| | | CTKQEPERNECFLQHKDDNPSLPPFERPEA |
| | | EAMCTSFKENPTTFMGHYLHEVARRHPYFY |
| | | APELLYYAEQYNEILTQCCAEADKESCLTP |
| | | KLDGVKEKALVSSVRQRMKCSSMQKFGERA |
| | | FKAWAVARLSQTFPNADFAEITKLATDLTK |
| | | VNKECCHGDLLECADDRAELAKYMCENQAT |
| | | ISSKLQTCCDKPLLKKAHCLSEVEHDTMPA |
| | | DLPAIAADFVEDQEVCKNYAEAKDVFLGTF |
| | | LYEYSRRHPDYSVSLLLRLAKKYEATLEKC |
| | | CAEANPPACYGTVLAEFQPLVEEPKNLVKT |
| | | NCDLYEKLGEYGFQNAILVRYTQKAPQVST |
| | | PTLVEAARNLGRVGTKCCTLPEDQRLPCVE |
| | | DYLSAILNRVCLLHEKTPVSEHVTKCCSGS |
| | | LVERRPCFSALTVDETYVPKEFKAETFTFH |
| | | SDICTLPEKEKQIKKQTALAELVKHKPKAT |
| | | AEQLKTVMDDFAQFLDTCCKAADKDTCFST |
| | | EGPNLVTRCKDALASGGPALFKSSFPPGSC |
| | | DLPQTHNLRNKRALTLLVQMRRLSPLSCLK |
| | | DRKDFGFPQEKVDAQQIKKAQAIPVLSELT |
| | | QQILNIFTSKDSSAAWNTTLLDSFCNDLHQ |
| | | QLNDLQGCLMQQVGVQEFPLTQEDALLAVR |
| | | KYFHRITVYLREKKHSPCAWEVVRAEVWRA |
| | | LSSSANVLGRLREEKSGGPALFKSSFPPGS |
| | | EVQLVESGGGLVQPGNSLRLSCAASGFTFS |
| | | KFGMSWVRQAPGKGLEWVSSISGSGRDTLY |
| | | AESVKGRFTISRDNAKTTLYLQMNSLRPED |
| | | TAVYYCTIGGSLSVSSQGTLVTVSSHHHHH |
| | | H |
| 17 | WW0821 | DAHKSEVAHRFKDLGEENFKALVLIAFAQY |
| | | LQQCPFEDHVKLVNEVTEFAKTCVADESAE |
| | | NCDKSLHTLFGDKLCTVATLRETYGEMADC |
| | | CAKQEPERNECFLQHKDDNPNLPRLVRPEV |
| | | DVMCTAFHDNEETFLKKYLYEIARRHPYFY |
| | | APELLFFAKRYKAAFTECCQAADKAACLLP |
| | | KLDELRDEGKASSAKQRLKCASLQKFGERA |
| | | FKAWAVARLSQRFPKAEFAEVSKLVTDLTK |
| | | VHTECCHGDLLECADDRADLAKYICENQDS |
| | | ISSKLKECCEKPLLEKSHCIAEVENDEMPA |
| | | DLPSLAADFVESKDVCKNYAEAKDVFLGMF |
| | | LYEYARRHPDYSVVLLLRLAKTYETTLEKC |
| | | CAAADPHECYAKVFDEFKPLVEEPQNLIKQ |
| | | NCELFEQLGEYKFQNALLVRYTKKVPQVST |
| | | PTLVEVSRNLGKVGSKCCKHPEAKRMPCAE |
| | | DYLSVVLNQLCVLHEKTPVSDRVTKCCTES |
| | | LVNRRPCFSALEVDETYVPKEFNAETFTFH |
| | | ADICTLSEKERQIKKQTALVELVKHKPKAT |
| | | KEQLKAVMDDFAAFVEKCCKADDKETCFAE |
| | | EGKKLVAASQAALGLSGGPALFKSSFPPGS |
| | | CDLPQTHSLGSRRTLMLLAQMRRISLFSCL |
| | | KDRHDFGFPQEEFGNQFQKAETIPVLHEMI |
| | | QQIFNLFSTKDSSAAWDETLLDKFYTELYQ |
| | | QLNDLEACVIQGVGVTETPLMKEDSILAVR |
| | | KYFQRITLYLKEKKYSPCAWEVVRAEIMRS |
| | | FSLSTNLQESLRSKESGGPALFKSSFPPGS |
| | | DAHKSEVAHRFKDLGEENFKALVLIAFAQY |
| | | LQQCPFEDHVKLVNEVTEFAKTCVADESAE |
| | | NCDKSLHTLFGDKLCTVATLRETYGEMADC |
| | | CAKQEPERNECFLQHKDDNPNLPRLVRPEV |
| | | DVMCTAFHDNEETFLKKYLYEIARRHPYFY |
| | | APELLFFAKRYKAAFTECCQAADKAACLLP |
| | | KLDELRDEGKASSAKQRLKCASLQKFGERA |
| | | FKAWAVARLSQRFPKAEFAEVSKLVTDLTK |
| | | VHTECCHGDLLECADDRADLAKYICENQDS |
| | | ISSKLKECCEKPLLEKSHCIAEVENDEMPA |
| | | DLPSLAADFVESKDVCKNYAEAKDVFLGMF |
| | | LYEYARRHPDYSVVLLLRLAKTYETTLEKC |
| | | CAAADPHECYAKVFDEFKPLVEEPQNLIKQ |
| | | NCELFEQLGEYKFQNALLVRYTKKVPQVST |
| | | PTLVEVSRNLGKVGSKCCKHPEAKRMPCAE |
| | | DYLSVVLNQLCVLHEKTPVSDRVTKCCTES |
| | | LVNRRPCFSALEVDETYVPKEFNAETFTFH |
| | | ADICTLSEKERQIKKQTALVELVKHKPKAT |
| | | KEQLKAVMDDFAAFVEKCCKADDKETCFAE |
| | | EGKKLVAASQAALGL |
| 18 | WW0834 | EVQLVESGGGLVQPGNSLRLSCAASGFTFS |
| | | KFGMSWVRQAPGKGLEWVSSISGSGRDTLY |
| | | AESVKGRFTISRDNAKTTLYLQMNSLRPED |
| | | TAVYYCTIGGSLSVSSQGTLVTVSSSGGPA |
| | | LFKSSFPPGSCDLPQTHSLGNRRALILLAQ |
| | | MGRISHFSCLKDRYDFGFPQEVFDGNQFQK |
| | | AQAISAFHEMIQQTFNLFSTKDSSAAWDET |
| | | LLDKFYIELFQQLNDLEACVTQEVGVEEIA |
| | | LMNEDSILAVRKYFQRITLYLMGKKYSPCA |
| | | WEVVRAEIMRSFSFSTNLQKGLRRKDSGGP |
| | | ALFKSSFPPGSEVQLVESGGGLVQPGNSLR |
| | | LSCAASGFTFSKFGMSWVRQAPGKGLEWVS |
| | | SISGSGRDTLYAESVKGRFTISRDNAKTTL |
| | | YLQMNSLRPEDTAVYYCTIGGSLSVSSQGT |
| | | LVTVSS |
| 19 | WW0742 | EVQLVESGGGLVQPGNSLRLSCAASGFTFS |
| | | KFGMSWVRQAPGKGLEWVSSISGSGRDTLY |
| | | AESVKGRFTISRDNAKTTLYLQMNSLRPED |
| | | TAVYYCTIGGSLSVSSQGTLVTVSSSGGPA |
| | | LFKSSFPPGSMSYNLLGFLQRSSNFQCQKL |
| | | LWQLNGRLEYCLKDRMNFDIPEEIKQLQQF |
| | | QKEDAALTIYEMLQNIFAIFRQDSSSTGWN |
| | | ETIVENLLANVYHQINHLKTVLEEKLEKED |
| | | FTRGKLMSSLHLKRYYGRILHYLKAKEYSH |
| | | CAWTIVRVEILRNFYFINRLTGYLRNSGGP |
| | | ALFKSSFPPGSEVQLVESGGGLVQPGNSLR |
| | | LSCAASGFTFSKFGMSWVRQAPGKGLEWVS |
| | | SISGSGRDTLYAESVKGRFTISRDNAKTTL |
| | | YLQMNSLRPEDTAVYYCTIGGSLSVSSQGT |
| | | LVTVSS |
| 20 | WW0612 | EVQLVESGGGLVQPGNSLRLSCAASGFTFS |
| | | KFGMSWVRQAPGKGLEWVSSISGSGRDTLY |
| | | AESVKGRFTISRDNAKTTLYLQMNSLRPED |
| | | TAVYYCTIGGSLSVSSQGTLVTVSSSGGPG |
| | | PAGLYAQPGSCDLPQTHSLGSRRTLMLLAQ |
| | | MRRISLFSCLKDRHDFGFPQEEFGNQFQKA |
| | | ETIPVLHEMIQQIFNLFSTKDSSAAWDETL |
| | | LDKFYTELYQQLNDLEACVIQGVGVTETPL |
| | | MKEDSILAVRKYFQRITLYLKEKKYSPCAW |
| | | EVVRAEIMRSFSLSTNLQESLRSKESGGPG |
| | | PAGLYAQPGSEVQLVESGGGLVQPGNSLRL |
| | | SCAASGFTFSKFGMSWVRQAPGKGLEWVSS |
| | | ISGSGRDTLYAESVKGRFTISRDNAKTTLY |
| | | LQMNSLRPEDTAVYYCTIGGSLSVSSQGTL |
| | | VTVSS |
| 21 | WW0045 | EVQLVESGGGLVQPGGSLRLSCAASGFTFS |
| | (Monomeric | SYTLAWVRQAPGKGLEWVAAIDSSSYTYSP |
| | IL-2 | DTVRGRFTISRDNAKNSLYLQMNSLRAEDT |
| | inducible | AVYYCARDSNWDALDYWGQGTTVTVSSGGG |
| | prodrug) | GSGGGGSGGGGSDIQMTQSPSSLSASVGDR |
| | | VTITCKASQNVGTNVGWYQQKPGKAPKALI |
| | | YSASFRYSGVPSRFSGSGSGTDFTLTISSL |
| | | QPEDFATYYCQQYYTYPYTFGGGTKVEIKG |
| | | GGGSGGGGSGGGGSGGGGSGGGGSGGGGSS |
| | | GGPGPAGMKGLPGSAPTSSSTKKTQLQLEH |
| | | LLLDLQMILNGINNYKNPKLTRMLTFKFYM |
| | | PKKATELKHLQCLEEELKPLEEVLNLAQSK |
| | | NFHLRPRDLISNINVIVLELKGSETTFMCE |
| | | YADETATIVEFLNRWITFCQSIISTLTSGG |
| | | PGPAGMKGLPGSEVQLVESGGGLVQPGNSL |
| | | RLSCAASGFTFSKFGMSWVRQAPGKGLEWV |
| | | SSISGSGRDTLYAESVKGRFTISRDNAKTT |
| | | LYLQMNSLRPEDTAVYYCTIGGSLSVSSQG |
| | | TLVTVSSHHHHHH |
| 22 | WW0046 | EVQLVESGGGLVQPGNSLRLSCAASGFTFS |
| | (Monomeric IL-2 | KFGMSWVRQAPGKGLEWVSSISGSGRDTLY |
| | inducible prodrug) | AESVKGRFTISRDNAKTTLYLQMNSLRPED |
| | | TAVYYCTIGGSLSVSSQGTLVTVSSSGGPG |
| | | PAGMKGLPGSEVQLVESGGGLVQPGGSLRL |
| | | SCAASGFTFSSYTLAWVRQAPGKGLEWVAA |
| | | IDSSSYTYSPDTVRGRFTISRDNAKNSLYL |
| | | QMNSLRAEDTAVYYCARDSNWDALDYWGQG |
| | | TTVTVSSGGGGSGGGGSGGGGSDIQMTQSP |
| | | SSLSASVGDRVTITCKASQNVGTNVGWYQQ |
| | | KPGKAPKALIYSASFRYSGVPSRFSGSGSG |
| | | TDFTLTISSLQPEDFATYYCQQYYTYPYTF |
| | | GGGTKVEIKGGGGSGGGGSGGGGSGGGGSG |
| | | GGGSGGGGSSGGPGPAGMKGLPGSAPTSSS |
| | | TKKTQLQLEHLLLDLQMILNGINNYKNPKL |
| | | TRMLTFKFYMPKKATELKHLQCLEEELKPL |
| | | EEVLNLAQSKNFHLRPRDLISNINVIVLEL |
| | | KGSETTFMCEYADETATIVEFLNRWITFCQ |
| | | SIISTLTHHHHHH |
| 23 | WW0203 | APTSSSTKKTQLQLEHLLLDLQMILNGINN |
| | (Monomeric IL-2 | YKNPKLTRMLTFKFYMPKKATELKHLQCLE |
| | inducible prodrug) | EELKPLEEVLNLAQSKNFHLRPRDLISNIN |
| | | VIVLELKGSETTFMCEYADETATIVEFLNR |
| | | WITFCQSIISTLTSGGPALFKSSFPPGSEV |
| | | QLVESGGGLVQPGNSLRLSCAASGFTFSKF |
| | | GMSWVRQAPGKGLEWVSSISGSGRDTLYAE |
| | | SVKGRFTISRDNAKTTLYLQMNSLRPEDTA |
| | | VYYCTIGGSLSVSSQGTLVTVSSGGGGSGG |
| | | GGSGGGGSGGGGSGGGGSGGGGSSGGPALF |
| | | KSSFPPGSEVQLVESGGGLVQPGGSLRLSC |
| | | AASGFTFSSYTLAWVRQAPGKGLEWVAAID |
| | | SSSYTYSPDTVRGRFTISRDNAKNSLYLQM |
| | | NSLRAEDTAVYYCARDSNWDALDYWGQGTT |
| | | VTVSSGGGGSGGGGSGGGGSDIQMTQSPSS |
| | | LSASVGDRVTITCKASQNVGTNVGWYQQKP |
| | | GKAPKALIYSASFRYSGVPSRFSGSGSGTD |
| | | FTLTISSLQPEDFATYYCQQYYTYPYTFGG |
| | | GTKVEIKHHHHHHEPEA |
| 24 | WW0204 | APTSSSTKKTQLQLEHLLLDLQMILNGINN |
| | (Monomeric IL-2 | YKNPKLTRMLTFKFYMPKKATELKHLQCLE |
| | inducible prodrug) | EELKPLEEVLNLAQSKNFHLRPRDLISNIN |
| | | VIVLELKGSETTFMCEYADETATIVEFLNR |
| | | WITFCQSIISTLTSGGPPLAQKLKSSPGSE |
| | | VQLVESGGGLVQPGNSLRLSCAASGFTFSK |
| | | FGMSWVRQAPGKGLEWVSSISGSGRDTLYA |
| | | ESVKGRFTISRDNAKTTLYLQMNSLRPEDT |
| | | AVYYCTIGGSLSVSSQGTLVTVSSGGGGSG |
| | | GGGSGGGGSGGGGSGGGGSGGGGSSGGPPL |
| | | AQKLKSSPGSEVQLVESGGGLVQPGGSLRL |
| | | SCAASGFTFSSYTLAWVRQAPGKGLEWVAA |
| | | IDSSSYTYSPDTVRGRFTISRDNAKNSLYL |
| | | QMNSLRAEDTAVYYCARDSNWDALDYWGQG |
| | | TTVTVSSGGGGSGGGGSGGGGSDIQMTQSP |
| | | SSLSASVGDRVTITCKASQNVGTNVGWYQQ |
| | | KPGKAPKALIYSASFRYSGVPSRFSGSGSG |
| | | TDFTLTISSLQPEDFATYYCQQYYTYPYTF |
| | | GGGTKVEIKHHHHHHEPEA |
| 25 | WW0205 | APTSSSTKKTQLQLEHLLLDLQMILNGINN |
| | (Monomeric IL-2 | YKNPKLTRMLTFKFYMPKKATELKHLQCLE |
| | inducible prodrug) | EELKPLEEVLNLAQSKNFHLRPRDLISNIN |
| | | VIVLELKGSETTFMCEYADETATIVEFLNR |
| | | WITFCQSIISTLTSGGPPGGPAGIGALFKS |
| | | SFPPLAQKLKSSPGSEVQLVESGGGLVQPG |
| | | NSLRLSCAASGFTFSKFGMSWVRQAPGKGL |
| | | EWVSSISGSGRDTLYAESVKGRFTISRDNA |
| | | KTTLYLQMNSLRPEDTAVYYCTIGGSLSVS |
| | | SQGTLVTVSSGGGGSGGGGSGGGGSGGGGS |
| | | GGGGSGGGGSSGGPPGGPAGIGALFKSSFP |
| | | PLAQKLKSSPGSEVQLVESGGGLVQPGGSL |
| | | RLSCAASGFTFSSYTLAWVRQAPGKGLEWV |
| | | AAIDSSSYTYSPDTVRGRFTISRDNAKNSL |
| | | YLQMNSLRAEDTAVYYCARDSNWDALDYWG |
| | | QGTTVTVSSGGGGSGGGGSGGGGSDIQMTQ |
| | | SPSSLSASVGDRVTITCKASQNVGTNVGWY |
| | | QQKPGKAPKALIYSASFRYSGVPSRFSGSG |
| | | SGTDFTLTISSLQPEDFATYYCQQYYTYPY |
| | | TFGGGTKVEIKHHHHHHEPEA |
| 26 | WW0234 | APTSSSTKKTQLQLEHLLLDLQMILNGINN |
| | (Monomeric IL-2 | YKNPKLTRMLTFKFYMPKKATELKHLQCLE |
| | inducible prodrug) | EELKPLEEVLNLAQSKNFHLRPRDLISNIN |
| | | VIVLELKGSETTFMCEYADETATIVEFLNR |
| | | WITFCQSIISTLTSGGPGPAGMKGLPGSEV |
| | | QLVESGGGLVQPGNSLRLSCAASGFTFSKF |
| | | GMSWVRQAPGKGLEWVSSISGSGRDTLYAE |
| | | SVKGRFTISRDNAKTTLYLQMNSLRPEDTA |
| | | VYYCTIGGSLSVSSQGTLVTVSSGGGGSGG |
| | | GGSGGGGSGGGGSGGGGSGGGGSSGGPGPA |
| | | GMKGLPGQVQLQESGGGLVQTGGSLRLSCT |
| | | TSGTIFSGYTMGWYRQAPGEQRELVAVISG |
| | | GGDTNYADSVKGRFTISRDNTKDTMYLQMN |
| | | SLKPEDTAVYYCYSREVTPPWKLYWGQGTQ |
| | | VTVSSAAAYPYDVPDYGSHHHHHH |
| 27 | WW0235 | APTSSSTKKTQLQLEHLLLDLQMILNGINN |
| | (Monomeric IL-2 | YKNPKLTRMLTFKFYMPKKATELKHLQCLE |
| | inducible prodrug) | EELKPLEEVLNLAQSKNFHLRPRDLISNIN |
| | | VIVLELKGSETTFMCEYADETATIVEFLNR |
| | | WITFCQSIISTLTSGGPGPAGMKGLPGSEV |
| | | QLVESGGGLVQPGNSLRLSCAASGFTFSKF |
| | | GMSWVRQAPGKGLEWVSSISGSGRDTLYAE |
| | | SVKGRFTISRDNAKTTLYLQMNSLRPEDTA |
| | | VYYCTIGGSLSVSSQGTLVTVSSGGGGSGG |
| | | GGSGGGGSGGGGSGGGGSGGGGSSGGPGPA |
| | | GMKGLPGQVQLQESGGGLVQEGGSLRLSCA |
| | | ASERIFSTDVMGWYRQAAEKQRELVAVVSA |
| | | RGTTNYLDAVKGRFTISRDNARNTLTLQMN |
| | | DLKPEDTASYYCYVRETTSPWRIYWGQGTQ |
| | | VTVSSAAAYPYDVPDYGSHHHHHH |
| 28 | WW0236 | APTSSSTKKTQLQLEHLLLDLQMILNGINN |
| | (Monomeric IL-2 | YKNPKLTRMLTFKFYMPKKATELKHLQCLE |
| | inducible prodrug) | EELKPLEEVLNLAQSKNFHLRPRDLISNIN |
| | | VIVLELKGSETTFMCEYADETATIVEFLNR |
| | | WITFCQSIISTLTSGGPGPAGMKGLPGSEV |
| | | QLVESGGGLVQPGNSLRLSCAASGFTFSKF |
| | | GMSWVRQAPGKGLEWVSSISGSGRDTLYAE |
| | | SVKGRFTISRDNAKTTLYLQMNSLRPEDTA |
| | | VYYCTIGGSLSVSSQGTLVTVSSGGGGSGG |
| | | GGSGGGGSGGGGSGGGGSGGGGSSGGPGPA |
| | | GMKGLPGQVQLQESGGGLVQAGGSLRLSCA |
| | | ASGSIFSANAMGWYRQAPGKQRELVAVISS |
| | | GGSTNYADSVKGRFTISRDNAKNTVYLQMN |
| | | SLKPEDTAVYYCMYSGSYYYTPNDYWGQGT |
| | | QVTVSSAAAYPYDVPDYGSHHHHHH |
| 29 | WW0308 | APTSSSTKKTQLQLEHLLLDLQMILNGINN |
| | (Monomeric IL-2 | YKNPKLTRMLTFKFYMPKKATELKHLQCLE |
| | inducible prodrug) | EELKPLEEVLNLAQSKNFHLRPRDLISNIN |
| | | VIVLELKGSETTFMCEYADETATIVEFLNR |
| | | WITFCQSIISTLTSGGPGPAGMKGLPGSEV |
| | | QLVESGGGLVQPGNSLRLSCAASGFTFSKF |
| | | GMSWVRQAPGKGLEWVSSISGSGRDTLYAE |
| | | SVKGRFTISRDNAKTTLYLQMNSLRPEDTA |
| | | VYYCTIGGSLSVSSQGTLVTVSSGGGGSGG |
| | | GGSGGGGSGGGGSGGGGSGGGGSSGGPGPA |
| | | GMKGLPGSEVQLVESGGGLVQPGGSLRLSC |
| | | AASGFTFSSYTLAWVRQAPGKGLEWVAAID |
| | | SSSYTYSPDTVRGRFTISRDNAKNSLYLQM |
| | | NSLRAEDTAVYYCARDSNWDALDYWGQGTT |
| | | VTVSSGGGGSGGGGSGGGGSDIQMTQSPSS |
| | | LSASVGDRVTITCKASQNVGTNVGWYQQKP |
| | | GQAPRLLIYSASFRYSGVPSRFSGSGSGTD |
| | | FTLTISSLQPEDFATYYCQQYYTYPYTFGG |
| | | GTKVEIKHHHHHH |
| 30 | WW0415 | APTSSSTKKTQLQLEHLLLDLQMILNGINN |
| | (Monomeric IL-2 | YKNPKLTRMLTFKFYMPKKATELKHLQCLE |
| | inducible prodrug) | EELKPLEEVLNLAQSKNFHLRPRDLISNIN |
| | | VIVLELKGSETTFMCEYADETATIVEFLNR |
| | | WITFCQSIISTLTSGGPGPAGLYAQPGSEV |
| | | QLVESGGGLVQPGNSLRLSCAASGFTFSKF |
| | | GMSWVRQAPGKGLEWVSSISGSGRDTLYAE |
| | | SVKGRFTISRDNAKTTLYLQMNSLRPEDTA |
| | | VYYCTIGGSLSVSSQGTLVTVSSGGGGSGG |
| | | GGSGGGGSGGGGSGGGGSGGGGSSGGPGPA |
| | | GLYAQPGSEVQLVESGGGLVQPGGSLRLSC |
| | | AASGFTFSSYTLAWVRQAPGKGLEWVAAID |
| | | SSSYTYSPDTVRGRFTISRDNAKNSLYLQM |
| | | NSLRAEDTAVYYCARDSNWDALDYWGQGTT |
| | | VTVSSGGGGSGGGGSGGGGSDIQMTQSPSS |
| | | LSASVGDRVTITCKAREKLWSAVAWYQQKP |
| | | GKAPKSLIYSASFRYSGVPSRFSGSGSGTD |
| | | FTLTISSLQPEDFATYYCQQYYTYPYTFGG |
| | | GTKVEIK |
| 31 | WW0706 | EVQLVESGGGLVQPGNSLRLSCAASGFTFS |
| | Monomeric IL-12 | KFGMSWVRQAPGKGLEWVSSISGSGRDTLY |
| | (chimeric) | AESVKGRFTISRDNAKTTLYLQMNSLRPED |
| | polypeptide, anti- | TAVYYCTIGGSLSVSSQGTLVTVSSsggpG |
| | HSA sdAb, Fab | PAGLYAQpgsiwelkkdvyvveldwypdap |
| | Blocker, 2 | gemvvltcdtpeedgitwtldqssevlgsg |
| | cleavage sites | ktltiqvkefgdagqytchkggevlshsll |
| | | llhkkedgiwstdilkdqkepknktflrce |
| | | aknysgrftcwwlttistdltfsvkssrgs |
| | | sdpqgvtcgaatlsaervrgdnkeyeysve |
| | | cqedsacpaaeeslpievmvdavhklkyen |
| | | ytssffirdiikpdppknlqlkplknsrqv |
| | | evsweypdtwstphsyfsltfcvqvqgksk |
| | | rekkdrvftdktsatvicrknasisvraqd |
| | | ryyssswsewasvpcsggggsggggsgggg |
| | | srvipvsgparclsqsrnllkttddmvkta |
| | | reklkhysctaedidheditrdqtstlktc |
| | | lplelhknesclatretssttrgsclppqk |
| | | tslmmtlclgsiyedlkmyqtefqainaal |
| | | qnhnhqqiildkgmlvaidelmqslnhnge |
| | | tlrqkppvgeadpyrvkmklcillhafstr |
| | | vvtinrvmgylssasggpGPAGLYAQpgsg |
| | | gggsggggggggggggsggggggggsQSVL |
| | | TQPPSVSGAPGQRVTISCSGSeSNIGSNdV |
| | | KWYQQLPGTAPKLLIYYNDQRPSGVPDRFS |
| | | GSKSGTSASLAITGLQAEDEADYYCQSYDR |
| | | YTHPALLFGTGTKVTVLgqpkaapsvtlfp |
| | | psseelqankatlvclisdfypgavtvawk |
| | | adsspvkagvetttpskqsnnkyaassyls |
| | | ltpeqwkshrsyscqvthegstvektvapt |
| | | ecs** |
| 32 | WW0707 | EVQLVESGGGLVQPGNSLRLSCAASGFTFS |
| | Monomeric IL-12 | KFGMSWVRQAPGKGLEWVSSISGSGRDTLY |
| | polypeptide, anti- | AESVKGRFTISRDNAKTTLYLQMNSLRPED |
| | HSA sdAb, Fab | TAVYYCTIGGSLSVSSQGTLVTVSSsggpG |
| | Blocker, 2 | PAGLYAQpgsiwelkkdvyvveldwypdap |
| | cleavage sites | gemvvltcdtpeedgitwtldqssevlgsg |
| | | ktltiqvkefgdagqytchkggevlshsll |
| | | llhkkedgiwstdilkdqkepknktflrce |
| | | aknysgrftcwwlttistdltfsvkssrgs |
| | | sdpqgvtcgaatlsaervrgdnkeyeysve |
| | | cqedsacpaaeeslpievmvdavhklkyen |
| | | ytssffirdiikpdppknlqlkplknsrqv |
| | | evsweypdtwstphsyfsltfcvqvqgksk |
| | | rekkdrvftdktsatvicrknasisvraqd |
| | | ryyssswsewasvpcsggggsggggggggs |
| | | rnlpvatpdpgmfpclhhsqnllravsnml |
| | | qkarqtlefypctseeidheditkdktstv |
| | | eaclpleltknesclnsretsfitngscla |
| | | srktsfmmalclssiyedlkmyqvefktmn |
| | | akllmdpkrqifldqnmlavidelmqalnf |
| | | nsetvpqkssleepdfyktkiklcillhaf |
| | | riravtidrvmsylnassggpGPAGLYAQp |
| | | gsggggggggggggsggggggggggggsQS |
| | | VLTQPPSVSGAPGQRVTISCSGSeSNIGSN |
| | | dVKWYQQLPGTAPKLLIYYNDQRPSGVPDR |
| | | FSGSKSGTSASLAITGLQAEDEADYYCQSY |
| | | DRYTHPALLFGTGTKVTVLgqpkaapsvtl |
| | | fppsseelqankatlvclisdfypgavtva |
| | | wkadsspvkagvetttpskqsnnkyaassy |
| | | lsltpeqwkshrsyscqvthegstvektva |
| | | ptecs** |
| 33 | WW0708 | EVQLVESGGGLVQPGNSLRLSCAASGFTFS |
| | Monomeric IL-12 | KFGMSWVRQAPGKGLEWVSSISGSGRDTLY |
| | (chimeric) | AESVKGRFTISRDNAKTTLYLQMNSLRPED |
| | polypeptide, anti- | TAVYYCTIGGSLSVSSQGTLVTVSSsggpG |
| | HSA sdAb, Fab | PAGLYAQpgsiwelkkdvyvveldwypdap |
| | Blocker, 2 | gemvvltcdtpeedgitwtldqssevlgsg |
| | cleavage sites | ktltiqvkefgdagqytchkggevlshsll |
| | | llhkkedgiwstdilkdqkepknktflrce |
| | | aknysgrftcwwlttistdltfsvkssrgs |
| | | sdpqgvtcgaatlsaervrgdnkeyeysve |
| | | cqedsacpaaeeslpievmvdavhklkyen |
| | | ytssffirdiikpdppknlqlkplknsrqv |
| | | evsweypdtwstphsyfsltfcvqvqgksk |
| | | rekkdrvftdktsatvicrknasisvraqd |
| | | ryyssswsewasvpcsggggsggggsgggg |
| | | srvipvsgparclsqsrnllkttddmvkta |
| | | reklkhysctaedidheditrdqtstlktc |
| | | lplelhknesclatretssttrgsclppqk |
| | | tslmmtlclgsiyedlkmyqtefqainaal |
| | | qnhnhqqiildkgmlvaidelmqslnhnge |
| | | tlrqkppvgeadpyrvkmklcillhafstr |
| | | vvtinrvmgylssasggpGPAGLYAQpgsg |
| | | gggsggggsggggggggggggsggggsQSV |
| | | LTQPPSVSGAPGQRVTISCSGSRSNIGeNT |
| | | VKWYQQLPGTAPKLLIYYNDQRPSGVPDRF |
| | | SGSKSGTSASLAITGLQAEDEADYYCQSYD |
| | | RYTHPALLFGTGTKVTVLgqpkaapsvtlf |
| | | ppsseelqankatlvclisdfypgavtvaw |
| | | kadsspvkagvetttpskqsnnkyaassyl |
| | | sltpeqwkshrsyscqvthegstvektvap |
| | | tecs** |
| 34 | WW0709 | EVQLVESGGGLVQPGNSLRLSCAASGFTFS |
| | Monomeric IL-12 | KFGMSWVRQAPGKGLEWVSSISGSGRDTLY |
| | polypeptide, anti- | AESVKGRFTISRDNAKTTLYLQMNSLRPED |
| | HSA sdAb, Fab | TAVYYCTIGGSLSVSSQGTLVTVSSsggpG |
| | Blocker, 2 | PAGLYAQpgsiwelkkdvyvveldwypdap |
| | cleavage sites | gemvvltcdtpeedgitwtldqssevlgsg |
| | | ktltiqvkefgdagqytchkggevlshsll |
| | | llhkkedgiwstdilkdqkepknktflrce |
| | | aknysgrftcwwlttistdltfsvkssrgs |
| | | sdpqgvtcgaatlsaervrgdnkeyeysve |
| | | cqedsacpaaeeslpievmvdavhklkyen |
| | | ytssffirdiikpdppknlqlkplknsrqv |
| | | evsweypdtwstphsyfsltfcvqvqgksk |
| | | rekkdrvftdktsatvicrknasisvraqd |
| | | ryyssswsewasvpcsggggsggggsgggg |
| | | srnlpvatpdpgmfpclhhsqnllravsnm |
| | | lqkarqtlefypctseeidheditkdktst |
| | | veaclpleltknesclnsretsfitngscl |
| | | asrktsfmmalclssiyedlkmyqvefktm |
| | | nakllmdpkrqifldqnmlavidelmqaln |
| | | fnsetvpqkssleepdfyktkiklcillha |
| | | friravtidrvmsylnassggpGPAGLYAQ |
| | | pgsggggggggggggsggggggggggggQS |
| | | LTQPPSVSGAPGQRVTISCSGSRSNIGeNT |
| | | VKWYQQLPGTAPKLLIYYNDQRPSGVPDRF |
| | | SGSKSGTSASLAITGLQAEDEADYYCQSYD |
| | | RYTHPALLFGTGTKVTVLgqpkaapsvtlf |
| | | ppsseelqankatlvclisdfypgavtvaw |
| | | kadsspvkagvetttpskqsnnkyaassyl |
| | | sltpeqwkshrsyscqvthegstvektvap |
| | | tec*** |
| 35 | WW0710 | EVQLVESGGGLVQPGNSLRLSCAASGFTFS |
| | Monomeric | KFGMSWVRQAPGKGLEWVSSISGSGRDTLY |
| | IL-12 | AESVKGRFTISRDNAKTTLYLQMNSLRPED |
| | (chimeric) | TAVYYCTIGGSLSVSSQGTLVTVSSsggpG |
| | polypeptide, | PAGLYAQpgsiwelkkdvyvveldwypdap |
| | anti-HSA | gemvvltcdtpeedgitwtldqssevlgsg |
| | sdAb, Fab | ktltiqvkefgdagqytchkggevlshsll |
| | Blocker, 2 | llhkkedgiwstdilkdqkepknktflrce |
| | cleavage | aknysgrftcwwlttistdltfsvkssrgs |
| | sites | sdpqgvtcgaatlsaervrgdnkeyeysve |
| | | cqedsacpaaeeslpievmvdavhklkyen |
| | | ytssffirdiikpdppknlqlkplknsrqv |
| | | evsweypdtwstphsyfsltfcvqvqgksk |
| | | rekkdrvftdktsatvicrknasisvraqd |
| | | ryyssswsewasvpcsggggsggggsgggg |
| | | srvipvsgparclsqsrnllkttddmvkta |
| | | reklkhysctaedidheditrdqtstlktc |
| | | lplelhknesclatretssttrgsclppqk |
| | | tslmmtlclgsiyedlkmyqtefqainaal |
| | | qnhnhqqiildkgmlvaidelmqslnhnge |
| | | tlrqkppvgeadpyrvkmklcillhafstr |
| | | vvtinrvmgylssasggpGPAGLYAQpgsg |
| | | gggsggggggggggggsggggggggsQSVL |
| | | TQPPSVSGAPGQRVTISCSGSRSNIGeeTV |
| | | KWYQQLPGTAPKLLIYYNDQRPSGVPDRFS |
| | | GSKSGTSASLAITGLQAEDEADYYCQSYDR |
| | | YTHPALLFGTGTKVTVLgqpkaapsvtlfp |
| | | psseelqankatlvclisdfypgavtvawk |
| | | adsspvkagvetttpskqsnnkyaassyls |
| | | ltpeqwkshrsyscqvthegstvektvapt |
| | | ecs** |
| 36 | WW0711 | EVQLVESGGGLVQPGNSLRLSCAASGFTFS |
| | Monomeric | KFGMSWVRQAPGKGLEWVSSISGSGRDTLY |
| | IL-12 | AESVKGRFTISRDNAKTTLYLQMNSLRPED |
| | polypeptide, | TAVYYCTIGGSLSVSSQGTLVTVSSsggpG |
| | anti-HSA | PAGLYAQpgsiwelkkdvyvveldwypdap |
| | sdAb, Fab | gemvvltcdtpeedgitwtldqssevlgsg |
| | Blocker, 2 | ktltiqvkefgdagqytchkggevlshsll |
| | cleavage | llhkkedgiwstdilkdqkepknktflrce |
| | sites | aknysgrftcwwlttistdltfsvkssrgs |
| | | sdpqgvtcgaatlsaervrgdnkeyeysve |
| | | cqedsacpaaeeslpievmvdavhklkyen |
| | | ytssffirdiikpdppknlqlkplknsrqv |
| | | evsweypdtwstphsyfsltfcvqvqgksk |
| | | rekkdrvftdktsatvicrknasisvraqd |
| | | ryyssswsewasvpcsggggsggggsgggg |
| | | srnlpvatpdpgmfpclhhsqnllravsnm |
| | | lqkarqtlefypctseeidheditkdktst |
| | | veaclpleltknesclnsretsfitngscl |
| | | asrktsfmmalclssiyedlkmyqvefktm |
| | | nakllmdpkrqifldqnmlavidelmqaln |
| | | fnsetvpqkssleepdfyktkiklcillha |
| | | friravtidrvmsylnassggpGPAGLYAQ |
| | | pgsggggggggggggsggggggggggggsQ |
| | | SVLTQPPSVSGAPGQRVTISCSGSRSNIGe |
| | | eTVKWYQQLPGTAPKLLIYYNDQRPSGVPD |
| | | RFSGSKSGTSASLAITGLQAEDEADYYCQS |
| | | YDRYTHPALLFGTGTKVTVLgqpkaapsvt |
| | | lfppsseelqankatlvclisdfypgavtv |
| | | awkadsspvkagvetttpskqsnnkyaass |
| | | ylsltpeqwkshrsyscqvthegstvektv |
| | | aptecs** |
| 37 | WW0712 | EVQLVESGGGLVQPGNSLRLSCAASGFTFS |
| | Monomeric | KFGMSWVRQAPGKGLEWVSSISGSGRDTLY |
| | IL-12 | AESVKGRFTISRDNAKTTLYLQMNSLRPED |
| | (chimeric) | TAVYYCTIGGSLSVSSQGTLVTVSSsggpA |
| | polypeptide, | LFKSSFPpgsiwelkkdvyvveldwypdap |
| | anti- | gemvvltcdtpeedgitwtldqssevlgsg |
| | HSA sdAb, | ktltiqvkefgdagqytchkggevlshsll |
| | Fab | llhkkedgiwstdilkdqkepknktflrce |
| | Blocker, 2 | aknysgrftcwwlttistdltfsvkssrgs |
| | cleavage | sdpqgvtcgaatlsaervrgdnkeyeysve |
| | sites | cqedsacpaaeeslpievmvdavhklkyen |
| | | ytssffirdiikpdppknlqlkplknsrqv |
| | | evsweypdtwstphsyfsltfcvqvqgksk |
| | | rekkdrvftdktsatvicrknasisvraqd |
| | | ryyssswsewasvpcsggggsggggsgggg |
| | | srvipvsgparclsqsrnllkttddmvkta |
| | | reklkhysctaedidheditrdqtstlktc |
| | | lplelhknesclatretssttrgsclppqk |
| | | tslmmtlclgsiyedlkmyqtefqainaal |
| | | qnhnhqqiildkgmlvaidelmqslnhnge |
| | | tlrqkppvgeadpyrvkmklcillhafstr |
| | | vvtinrvmgylssasggpALFKSSFPpgsg |
| | | gggsggggsggggggggsggggggggsQSV |
| | | LTQPPSVSGAPGQRVTISCSGSRSNIGSNT |
| | | VKWYQQLPGTAPKLLIYYNDQRPSGVPDRF |
| | | SGSKSGTSASLAITGLQAEDEADYYCQSYD |
| | | RYTHPALLFGTGTKVTVLgqpkaapsvtlf |
| | | ppsseelqankatlvclisdfypgavtvaw |
| | | kadsspvkagvetttpskqsnnkyaassyl |
| | | sltpeqwkshrsyscqvthegstvektvap |
| | | tecs** |
| 38 | WW0713 | EVQLVESGGGLVQPGNSLRLSCAASGFTFS |
| | Monomeric | KFGMSWVRQAPGKGLEWVSSISGSGRDTLY |
| | IL-12 | AESVKGRFTISRDNAKTTLYLQMNSLRPED |
| | polypeptide, | TAVYYCTIGGSLSVSSQGTLVTVSSsggpA |
| | anti- | LFKSSFPpgsiwelkkdvyvveldwypdap |
| | HSA sdAb, | gemvvltcdtpeedgitwtldqssevlgsg |
| | Fab | ktltiqvkefgdagqytchkggevlshsll |
| | Blocker, 2 | llhkkedgiwstdilkdqkepknktflrce |
| | cleavage | aknysgrftcwwlttistdltfsvkssrgs |
| | sites | sdpqgvtcgaatlsaervrgdnkeyeysve |
| | | cqedsacpaaeeslpievmvdavhklkyen |
| | | ytssffirdiikpdppknlqlkplknsrqv |
| | | evsweypdtwstphsyfsltfcvqvqgksk |
| | | rekkdrvftdktsatvicrknasisvraqd |
| | | ryyssswsewasvpcsggggsggggsgggg |
| | | srnlpvatpdpgmfpclhhsqnllravsnm |
| | | lqkarqtlefypctseeidheditkdktst |
| | | veaclpleltknesclnsretsfitngscl |
| | | asrktsfmmalclssiyedlkmyqvefktm |
| | | nakllmdpkrqifldqnmlavidelmqaln |
| | | fnsetvpqkssleepdfyktkiklcillha |
| | | friravtidrvmsylnassggpALFKSSFP |
| | | pgsggggsggggggggsggggggggggggs |
| | | QSVLTQPPSVSGAPGQRVTISCSGSRSNIG |
| | | SNTVKWYQQLPGTAPKLLIYYNDQRPSGVP |
| | | DRFSGSKSGTSASLAITGLQAEDEADYYCQ |
| | | SYDRYTHPALLFGTGTKVTVLgqpkaapsv |
| | | tlfppsseelqankatlvclisdfypgavt |
| | | vawkadsspvkagvetttpskqsnnkyaas |
| | | sylsltpeqwkshrsyscqvthegstvekt |
| | | vaptecs** |
| 39 | WW0714 | EVQLVESGGGLVQPGNSLRLSCAASGFTFS |
| | Monomeric | KFGMSWVRQAPGKGLEWVSSISGSGRDTLY |
| | IL-12 | AESVKGRFTISRDNAKTTLYLQMNSLRPED |
| | (chimeric) | TAVYYCTIGGSLSVSSQGTLVTVSSsggpA |
| | polypeptide, | LFKSSFPpgsiwelkkdvyvveldwypdap |
| | anti- | gemvvltcdtpeedgitwtldqssevlgsg |
| | HSA sdAb, | ktltiqvkefgdagqytchkggevlshsll |
| | Fab | llhkkedgiwstdilkdqkepknktflrce |
| | Blocker, 2 | aknysgrftcwwlttistdltfsvkssrgs |
| | cleavage | sdpqgvtcgaatlsaervrgdnkeyeysve |
| | sites | cqedsacpaaeeslpievmvdavhklkyen |
| | | ytssffirdiikpdppknlqlkplknsrqv |
| | | evsw |
| | | eypdtwstphsyfsltfcvqvqgkskrekk |
| | | drvftdktsatvicrknasisvraqdryys |
| | | sswsewasvpcsggggsggggsggggsrvi |
| | | pvsgparclsqsrnllkttddmvktarekl |
| | | khysctaedidheditrdqtstlktclple |
| | | lhknesclatretssttrgsclppqktslm |
| | | mtlclgsiyedlkmyqtefqainaalqnhn |
| | | hqqiildkgmlvaidelmqslnhngetlrq |
| | | kppvgeadpyrvkmklcillhafstrvvti |
| | | nrvmgylssasggpALFKSSFPpgsggggs |
| | | ggggsggggsggggggggggggsQSVLTQP |
| | | PSVSGAPGQRVTISCSGSRSNIGSeTVKWY |
| | | QQLPGTAPKLLIYYNDQRPSGVPDRFSGSK |
| | | SGTSASLAITGLQAEDEADYYCQSYDRYTH |
| | | PALLFGTGTKVTVLgqpkaapsvtlfppss |
| | | eelqankatlvclisdfypgavtvawkads |
| | | spvkagvetttpskqsnnkyaassylsltp |
| | | eqwkshrsyscqvthegstvektvaptecs |
| | | ** |
| 40 | WW0715 | EVQLVESGGGLVQPGNSLRLSCAASGFTFS |
| | Monomeric | KFGMSWVRQAPGKGLEWVSSISGSGRDTLY |
| | IL-12 | AESVKGRFTISRDNAKTTLYLQMNSLRPED |
| | polypeptide, | TAVYYCTIGGSLSVSSQGTLVTVSSsggpA |
| | anti- | LFKSSFPpgsiwelkkdvyvveldwypdap |
| | HSA sdAb, | gemvvltcdtpeedgitwtldqssevlgsg |
| | Fab | ktltiqvkefgdagqytchkggevlshsll |
| | Blocker, 2 | llhkkedgiwstdilkdqkepknktflrce |
| | cleavage | aknysgrftcwwlttistdltfsvkssrgs |
| | sites | sdpqgvtcgaatlsaervrgdnkeyeysve |
| | | cqedsacpaaeeslpievmvdavhklkyen |
| | | ytssffirdiikpdppknlqlkplknsrqv |
| | | evsweypdtwstphsyfsltfcvqvqgksk |
| | | rekkdrvftdktsatvicrknasisvraqd |
| | | ryyssswsewasvpcsggggsggggsgggg |
| | | srnlpvatpdpgmfpclhhsqnllravsnm |
| | | lqkarqtlefypctseeidheditkdktst |
| | | veaclpleltknesclnsretsfitngscl |
| | | asrktsfmmalclssiyedlkmyqvefktm |
| | | nakllmdpkrqifldqnmlavidelmqaln |
| | | fnsetvpqkssleepdfyktkiklcillha |
| | | friravtidrvmsylnassggpALFKSSFP |
| | | pgsggggsggggggggggggsggggggggs |
| | | QSVLTQPPSVSGAPGQRVTISCSGSRSNIG |
| | | SeTVKWYQQLPGTAPKLLIYYNDQRPSGVP |
| | | DRFSGSKSGTSASLAITGLQAEDEADYYCQ |
| | | SYDRYTHPALLFGTGTKVTVLgqpkaapsv |
| | | tlfppsseelqankatlvclisdfypgavt |
| | | vawkadsspvkagvetttpskqsnnkyaas |
| | | sylsltpeqwkshrsyscqvthegstvekt |
| | | vaptecs** |
| 41-194 | | Place hold SEQ ID NOs.: 41-194 |
| 195 | MMP14 1 | GPAGLYAQ |
| 196 | MMP9 1 | GPAGMKGL |
| 197 | FAPa 1 | PGGPAGIG |
| 198 | CTSL1_1 | ALFKSSFP |
| 199 | CTSL1 2 | ALFFSSPP |
| 200 | ADAM17 1 | LAQRLRSS |
| 201 | ADAM17_2 | LAQKLKSS |
| 202 | ALU30-1 | GALFKSSFPSGGGPAGLYAQGGSGKGGSGK |
| 203 | ALU30-2 | RGSGGGPAGLYAQGSGGGPAGLYAQGGSGK |
| 204 | ALU30-3 | KGGGPAGLYAQGPAGLYAQGPAGLYAQGSR |
| 205 | ALU30-4 | RGGPAGLYAQGGPAGLYAQGGGPAGLYAQK |
| 206 | ALU30-5 | KGGALFKSSFPGGPAGIGPLAQKLKSSGGS |
| 207 | ALU30-6 | SGGPGGPAGIGALFKSSFPLAQKLKSSGGG |
| 208 | ALU30-7 | RGPLAQKLKSSALFKSSFPGGPAGIGGGGK |
| 209 | ALU30-8 | GGGALFKSSFPLAQKLKSSPGGPAGIGGGR |
| 210 | ALU30-9 | RGPGGPAGIGPLAQKLKSSALFKSSFPGGG |
| 211 | ALU30-10 | RGGPLAQKLKSSPGGPAGIGALFKSSFPGK |
| 212 | ALU30-11 | RSGGPAGLYAQALFKSSFPLAQKLKSSGGG |
| 213 | ALU30-12 | GGPLAQKLKSSALFKSSFPGPAGLYAQGGR |
| 214 | ALU30-13 | GGALFKSSFPGPAGLYAQPLAQKLKSSGGK |
| 215 | ALU30-14 | RGGALFKSSFPLAQKLKSSGPAGLYAQGGK |
| 216 | ALU30-15 | RGGGPAGLYAQPLAQKLKSSALFKSSFPGG |
| 217 | ALU30-16 | SGPLAQKLKSSGPAGLYAQALFKSSFPGSK |
| 218 | ALU30-17 | KGGPGGPAGIGPLAQRLRSSALFKSSFPGR |
| 219 | ALU30-18 | KSGPGGPAGIGALFFSSPPLAQKLKSSGGR |
| 220 | ALU30-19 | SGGFPRSGGSFNPRTFGSKRKRRGSRGGGG |
| 221 | MMP14 substrate | GPLGLKAQ |
| | motif sequence | |
| 222 | MMP14 substrate | LPLGLKAQ |
| | motif sequence | |
| 223 | MMP14 substrate | SPLGLKAQ |
| | motif sequence | |
| 224 | MMP14 substrate | QPLGLKAQ |
| | motif sequence | |
| 225 | MMP14 substrate | KPLGLKAQ |
| | motif sequence | |
| 226 | MMP14 substrate | FPLGLKAQ |
| | motif sequence | |
| 227 | MMP14 substrate | HPLGLKAQ |
| | motif sequence | |
| 228 | MMP14 substrate | PPLGLKAQ |
| | motif sequence | |
| 229 | MMP14 substrate | APLGLKAQ |
| | motif sequence | |
| 230 | MMP14 substrate | DPLGLKAQ |
| | motif sequence | |
| 231 | MMP14 substrate | GPHGLKAQ |
| | motif sequence | |
| 232 | MMP14 substrate | GPSGLKAQ |
| | motif sequence | |
| 233 | MMP14 substrate | GPQGLKAQ |
| | motif sequence | |
| 234 | MMP14 substrate | GPPGLKAQ |
| | motif sequence | |
| 235 | MMP14 substrate | GPEGLKAQ |
| | motif sequence | |
| 236 | MMP14 substrate | GPFGLKAQ |
| | motif sequence | |
| 237 | MMP14 substrate | GPRGLKAQ |
| | motif sequence | |
| 238 | MMP14 substrate | GPGGLKAQ |
| | motif sequence | |
| 239 | MMP14 substrate | GPAGLKAQ |
| | motif sequence | |
| 240 | MMP14 substrate | LPAGLKGA |
| | motif sequence | |
| 241 | MMP14 substrate | GPAGLYAQ |
| | motif sequence | |
| 242 | MMP14 substrate | GPANLVAQ |
| | motif sequence | |
| 243 | MMP14 substrate | GPAALVGA |
| | motif sequence | |
| 244 | MMP14 substrate | GPANLRAQ |
| | motif sequence | |
| 245 | MMP14 substrate | GPAGLRAQ |
| | motif sequence | |
| 246 | MMP14 substrate | GPAGLVAQ |
| | motif sequence | |
| 247 | MMP14 substrate | GPAGLRGA |
| | motif sequence | |
| 248 | MMP14 substrate | LPAGLVGA |
| | motif sequence | |
| 249 | MMP14 substrate | GPAGLKGA |
| | motif sequence | |
| 250 | MMP14 substrate | GPLALKAQ |
| | motif sequence | |
| 251 | MMP14 substrate | GPLNLKAQ |
| | motif sequence | |
| 252 | MMP14 substrate | GPLHLKAQ |
| | motif sequence | |
| 253 | MMP14 substrate | GPLYLKAQ |
| | motif sequence | |
| 254 | MMP14 substrate | GPLPLKAQ |
| | motif sequence | |
| 255 | MMP14 substrate | GPLELKAQ |
| | motif sequence | |
| 256 | MMP14 substrate | GPLRLKAQ |
| | motif sequence | |
| 257 | MMP14 substrate | GPLLLKAQ |
| | motif sequence | |
| 258 | MMP14 substrate | GPLSLKAQ |
| | motif sequence | |
| 259 | MMP14 substrate | GPLGLYAQ |
| | motif sequence | |
| 260 | MMP14 substrate | GPLGLFAQ |
| | motif sequence | |
| 261 | MMP14 substrate | GPLGLLAQ |
| | motif sequence | |
| 262 | MMP14 substrate | GPLGLHAQ |
| | motif sequence | |
| 263 | MMP14 substrate | GPLGLRAQ |
| | motif sequence | |
| 264 | MMP14 substrate | GPLGLAAQ |
| | motif sequence | |
| 265 | MMP14 substrate | GPLGLEAQ |
| | motif sequence | |
| 266 | MMP14 substrate | GPLGLGAQ |
| | motif sequence | |
| 267 | MMP14 substrate | GPLGLPAQ |
| | motif sequence | |
| 268 | MMP14 substrate | GPLGLQAQ |
| | motif sequence | |
| 269 | MMP14 substrate | GPLGLSAQ |
| | motif sequence | |
| 270 | MMP14 substrate | GPLGLVAQ |
| | motif sequence | |
| 271 | MMP14 substrate | GPLGLKLQ |
| | motif sequence | |
| 272 | MMP14 substrate | GPLGLKFQ |
| | motif sequence | |
| 273 | MMP14 substrate | GPLGLKEQ |
| | motif sequence | |
| 274 | MMP14 substrate | GPLGLKKQ |
| | motif sequence | |
| 275 | MMP14 substrate | GPLGLKQQ |
| | motif sequence | |
| 276 | MMP14 substrate | GPLGLKSQ |
| | motif sequence | |
| 277 | MMP14 substrate | GPLGLKGQ |
| | motif sequence | |
| 278 | MMP14 substrate | GPLGLKHQ |
| | motif sequence | |
| 279 | MMP14 substrate | GPLGLKPQ |
| | motif sequence | |
| 280 | MMP14 substrate | GPLGLKAG |
| | motif sequence | |
| 281 | MMP14 substrate | GPLGLKAF |
| | motif sequence | |
| 282 | MMP14 substrate | GPLGLKAP |
| | motif sequence | |
| 283 | MMP14 substrate | GPLGLKAL |
| | motif sequence | |
| 284 | MMP14 substrate | GPLGLKAE |
| | motif sequence | |
| 285 | MMP14 substrate | GPLGLKAA |
| | motif sequence | |
| 286 | MMP14 substrate | GPLGLKAH |
| | motif sequence | |
| 287 | MMP14 substrate | GPLGLKAK |
| | motif sequence | |
| 288 | MMP14 substrate | GPLGLKAS |
| | motif sequence | |
| 289 | MMP14 substrate | GPLGLFGA |
| | motif sequence | |
| 290 | MMP14 substrate | GPLGLQGA |
| | motif sequence | |
| 291 | MMP14 substrate | GPLGLVGA |
| | motif sequence | |
| 292 | MMP14 substrate | GPLGLAGA |
| | motif sequence | |
| 293 | MMP14 substrate | GPLGLLGA |
| | motif sequence | |
| 294 | MMP14 substrate | GPLGLRGA |
| | motif sequence | |
| 295 | MMP14 substrate | GPLGLYGA |
| | motif sequence | |
| 296 | CTSL1 substrate | ALFKSSPP |
| | motif sequence | |
| 297 | CTSL1 substrate | SPFRSSRQ |
| | motif sequence | |
| 298 | CTSL1 substrate | KLFKSSPP |
| | motif sequence | |
| 299 | CTSL1 substrate | HLFKSSPP |
| | motif sequence | |
| 300 | CTSLI substrate | SLFKSSPP |
| | motif sequence | |
| 301 | CTSL1 substrate | QLFKSSPP |
| | motif sequence | |
| 302 | CTSL1 substrate | LLFKSSPP |
| | motif sequence | |
| 303 | CTSL1 substrate | PLFKSSPP |
| | motif sequence | |
| 304 | CTSL1 substrate | FLFKSSPP |
| | motif sequence | |
| 305 | CTSL1 substrate | GLFKSSPP |
| | motif sequence | |
| 306 | CTSLI substrate | VLFKSSPP |
| | motif sequence | |
| 307 | CTSL1 substrate | ELFKSSPP |
| | motif sequence | |
| 308 | CTSL1 substrate | AKFKSSPP |
| | motif sequence | |
| 309 | CTSL1 substrate | AHFKSSPP |
| | motif sequence | |
| 310 | CTSL1 substrate | AGFKSSPP |
| | motif sequence | |
| 311 | CTSL1 substrate | APFKSSPP |
| | motif sequence | |
| 312 | CTSL1 substrate | ANFKSSPP |
| | motif sequence | |
| 313 | CTSL1 substrate | AFFKSSPP |
| | motif sequence | |
| 314 | CTSL1 substrate | AAFKSSPP |
| | motif sequence | |
| 315 | CTSL1 substrate | ASFKSSPP |
| | motif sequence | |
| 316 | CTSL1 substrate | AEFKSSPP |
| | motif sequence | |
| 317 | CTSL1 substrate | ALRKSSPP |
| | motif sequence | |
| 318 | CTSL1 substrate | ALLKSSPP |
| | motif sequence | |
| 319 | CTSL1 substrate | ALAKSSPP |
| | motif sequence | |
| 320 | CTSL1 substrate | ALQKSSPP |
| | motif sequence | |
| 321 | CTSL1 substrate | ALHKSSPP |
| | motif sequence | |
| 322 | CTSL1 substrate | ALPKSSPP |
| | motif sequence | |
| 323 | CTSL1 substrate | ALTKSSPP |
| | motif sequence | |
| 324 | CTSL1 substrate | ALGKSSPP |
| | motif sequence | |
| 325 | CTSL1 substrate | ALDKSSPP |
| | motif sequence | |
| 326 | CTSL1 substrate | ALFFSSPP |
| | motif sequence | |
| 327 | CTSL1 substrate | ALFHSSPP |
| | motif sequence | |
| 328 | CTSL1 substrate | ALFTSSPP |
| | motif sequence | |
| 329 | CTSL1 substrate | ALFASSPP |
| | motif sequence | |
| 330 | CTSL1 substrate | ALFQSSPP |
| | motif sequence | |
| 331 | CTSL1 substrate | ALFLSSPP |
| | motif sequence | |
| 332 | CTSL1 substrate | ALFGSSPP |
| | motif sequence | |
| 333 | CTSL1 substrate | ALFESSPP |
| | motif sequence | |
| 334 | CTSLI substrate | ALFPSSPP |
| | motif sequence | |
| 335 | CTSL1 substrate | ALFKHSPP |
| | motif sequence | |
| 336 | CTSL1 substrate | ALFKLSPP |
| | motif sequence | |
| 337 | CTSL1 substrate | ALFKKSPP |
| | motif sequence | |
| 338 | CTSL1 substrate | ALFKASPP |
| | motif sequence | |
| 339 | CTSL1 substrate | ALFKISPP |
| | motif sequence | |
| 340 | CTSL1 substrate | ALFKGSPP |
| | motif sequence | |
| 341 | CTSL1 substrate | ALFKNSPP |
| | motif sequence | |
| 342 | CTSL1 substrate | ALFKRSPP |
| | motif sequence | |
| 343 | CTSL1 substrate | ALFKESPP |
| | motif sequence | |
| 344 | CTSL1 substrate | ALFKFSPP |
| | motif sequence | |
| 345 | CTSL1 substrate | ALFKPSPP |
| | motif sequence | |
| 346 | CTSLI substrate | ALFKSFPP |
| | motif sequence | |
| 347 | CTSL1 substrate | ALFKSLPP |
| | motif sequence | |
| 348 | CTSL1 substrate | ALFKSIPP |
| | motif sequence | |
| 349 | CTSLI substrate | ALFKSKPP |
| | motif sequence | |
| 350 | CTSL1 substrate | ALFKSAPP |
| | motif sequence | |
| 351 | CTSL1 substrate | ALFKSQPP |
| | motif sequence | |
| 352 | CTSL1 substrate | ALFKSPPP |
| | motif sequence | |
| 353 | CTSL1 substrate | ALFKSEPP |
| | motif sequence | |
| 354 | CTSL1 substrate | ALFKSGPP |
| | motif sequence | |
| 355 | CTSL1 substrate | ALFKSSFP |
| | motif sequence | |
| 356 | CTSL1 substrate | ALFKSSLP |
| | motif sequence | |
| 357 | CTSL1 substrate | ALFKSSGP |
| | motif sequence | |
| 358 | CTSL1 substrate | ALFKSSSP |
| | motif sequence | |
| 359 | CTSL1 substrate | ALFKSSVP |
| | motif sequence | |
| 360 | CTSL1 substrate | ALFKSSHP |
| | motif sequence | |
| 361 | CTSL1 substrate | ALFKSSAP |
| | motif sequence | |
| 362 | CTSL1 substrate | ALFKSSNP |
| | motif sequence | |
| 363 | CTSL1 substrate | ALFKSSKP |
| | motif sequence | |
| 364 | CTSL1 substrate | ALFKSSEP |
| | motif sequence | |
| 365 | CTSL1 substrate | ALFKSSPF |
| | motif sequence | |
| 366 | CTSL1 substrate | ALFKSSPH |
| | motif sequence | |
| 367 | CTSL1 substrate | ALFKSSPG |
| | motif sequence | |
| 368 | CTSL1 substrate | ALFKSSPA |
| | motif sequence | |
| 369 | CTSL1 substrate | ALFKSSPS |
| | motif sequence | |
| 370 | CTSL1 substrate | ALFKSSPV |
| | motif sequence | |
| 371 | CTSL1 substrate | ALFKSSPQ |
| | motif sequence | |
| 372 | CTSL1 substrate | ALFKSSPK |
| | motif sequence | |
| 373 | CTSL1 substrate | ALFKSSPL |
| | motif sequence | |
| 374 | CTSL1 substrate | ALFKSSPD |
| | motif sequence | |
| 375 | MMP7 | KRALGLPG |
| 376 | MMP7 | (DE)8RPLALWRS(DR)8 |
| | MMP9 | PR(S/T)(L/I)(S/T) |
| 378 | MMP9 | LEATA |
| 379 | MMP11 | GGAANLVRGG |
| 380 | MMP14 | SGRIGFLRTA |
| 381 | MMP | PLGLAG |
| 382 | MMP | PLGLAX |
| 383 | MMP | PLGC(me)AG |
| 384 | MMP | ESPAYYTA |
| 385 | MMP | RLQLKL |
| 386 | MMP | RLQLKAC |
| 387 | MMP2, MMP9, | EP(Cit)G(Hof) YL |
| | MMP14 | |
| 388 | Urokinase | SGRSA |
| | plasminogen | |
| | activator | |
| | (uPA) | |
| 389 | Urokinase | DAFK |
| | plasminogen | |
| | activator | |
| | (uPA) | |
| 390 | Urokinase | GGGRR |
| | plasminogen | |
| | activator (uPA) | |
| 391 | Lysosomal | GFLG |
| | Enzyme | |
| 392 | Lysosomal | ALAL |
| | Enzyme | |
| | Lysosomal | FK |
| | Enzyme | |
| | Cathepsin B | NLL |
| 395 | Cathepsin D | PIC(Et)FF |
| 396 | Cathepsin K | GGPRGLPG |
| 397 | Prostate | HSSKLQ |
| | Specific | |
| | Antigen | |
| 398 | Prostate | HSSKLQL |
| | Specific | |
| | Antigen | |
| 399 | Prostate | HSSKLQEDA |
| | Specific | |
| | Antigen | |
| 400 | Herpes Simplex | LVLASSSFGY |
| | Virus Protease | |
| 401 | HIV Protease | GVSQNYPIVG |
| 402 | CMV Protease | GVVQASCRLA |
| | Thrombin | F(Pip)RS |
| 404 | Thrombin | DPRSFL |
| 405 | Thrombin | PPRSFL |
| 406 | Caspase-3 | DEVD |
| 407 | Caspase-3 | DEVDP |
| 408 | Caspase-3 | KGSGDVEG |
| 409 | Interleukin | GWEHDG |
| | 1β | |
| | converting | |
| | enzyme | |
| 410 | Enterokinase | EDDDDKA |
| 411 | FAP | KQEQNPGST |
| 412 | Kallikrein 2 | GKAFRR |
| 413 | Plasmin | DAFK |
| 414 | Plasmin | DVLK |
| 415 | Plasmin | DAFK |
| 416 | TOP | ALLLALL |
| 417 | | GPLGVRG |
| 418 | | IPVSLRSG |
| 419 | | VPLSLYSG |
| 420 | | SGESPAYYTA |
| 421 | WW0757 | EVQLVESGGGLVQPGNSLRLSCAASGFTFS |
| | | KFGMSWVRQAPGKGLEWVSSISGSGRDTLY |
| | | AESVKGRFTISRDNAKTTLYLQMNSLRPED |
| | | TAVYYCTIGGSLSVSSQGTLVTVSSsggpA |
| | | LFKSSFPpgsrvipvsgparclsqsrnllk |
| | | ttddmvktareklkhysctaedidheditr |
| | | dqtstlktclplelhknesclatretsstt |
| | | rgsclppqktsImmtlclgsiyedikmyqt |
| | | efqainaalqnhnhqqiildkgmlvaidel |
| | | mqsInhngetlrqkppvgeadpyrvkmklc |
| | | illhafstrvvtinrvmgylssasggpALF |
| | | KSSFPpgsggggsggggsggggsggggsgg |
| | | ggsggggsQSVLTQPPSVSGAPGQRVTISC |
| | | SGSRSNIGSNTVKWYQQLPGTAPKLLIYYN |
| | | DQRPSGVPDRFSGSKSGTSASLAITGLQAE |
| | | DEADYYCQSYDRYTHPALLFGTGTKVTVLg |
| | | gggsggggsggggsQVQLVESGGGVVQPGR |
| | | SLRLSCAASGFTFSSYGMHWVRQAPGKGLE |
| | | WVAFIRYeGSNKYYAeSVKGRFTISRDNSK |
| | | NTLYLQMNSLRAEDTAVYYCKTHGSHDNWG |
| | | QGTMVTVSS |
| 422 | WW0610 | EVQLVESGGGLVQPGNSLRLSCAASGFTFS |
| | | KFGMSWVRQAPGKGLEWVSSISGSGRDTLY |
| | | AESVKGRFTISRDNAKTTLYLQMNSLRPED |
| | | TAVYYCTIGGSLSVSSQGTLVTVSSSGGPA |
| | | LFKSSFPPGSCDLPQTHNLRNKRALTLLVQ |
| | | MRRLSPLSCLKDRKDFGFPQEKVDAQQIKK |
| | | AQAIPVLSELTQQILNIFTSKDSSAAWNTT |
| | | LLDSFCNDLHQQLNDLQGCLMQQVGVQEFP |
| | | LTQEDALLAVRKYFHRITVYLREKKHSPCA |
| | | WEVVRAEVWRALSSSANVLGRLREEKSGGP |
| | | ALFKSSFPPGSEVQLVESGGGLVQPGNSLR |
| | | LSCAASGFTFSKFGMSWVRQAPGKGLEWVS |
| | | SISGSGRDTLYAESVKGRFTISRDNAKTTL |
| | | YLQMNSLRPEDTAVYYCTIGGSLSVSSQGT |
| | | LVTVSS |

1. A combination comprising a therapeutically effective amount of (i) an inducible cytokine prodrug comprising a cytokine polypeptide [A], a blocking element [D], optionally a half-life extension element [H] and a protease-cleavable polypeptide linker; and (ii) an anti-PD-1 antibody or an anti-PD-L1 antibody for use in treating a cancer in a subject in need thereof;
wherein the cytokine polypeptide and the cytokine blocking element and the optional half-life extension element when present are operably linked by the protease-cleavable polypeptide linker and the inducible cytokine prodrug has attenuated cytokine receptor activating activity, wherein the cytokine-receptor activating activity of the inducible cytokine prodrug is at least about 10× less than the cytokine receptor activating activity of the polypeptide that contains the cytokine polypeptide that is produced by cleavage of the protease cleavable linker.

2. A method of treating a cancer in a subject comprising administering to the subject in need thereof a therapeutically effective amount of (i) an inducible cytokine prodrug comprising a cytokine polypeptide [A], a blocking element [D], optionally a half-life extension element [H] and a protease-cleavable polypeptide linker; and (ii) an anti-PD-1 antibody or an anti-PD-L1 antibody;
wherein the cytokine polypeptide and the cytokine blocking element and the optional half-life extension element when present are operably linked by the protease-cleavable polypeptide linker and the inducible cytokine prodrug has attenuated cytokine receptor activating activity, wherein the cytokine-receptor activating activity of the inducible cytokine prodrug is at least about 10× less than the cytokine receptor activating activity of the polypeptide that contains the cytokine polypeptide that is produced by cleavage of the protease cleavable linker.

3. The use of claim 1 or the method of claim 2, wherein the cytokine polypeptide is IL-2, IL-12, interferon alpha, interferon beta, interferon gamma, or a mutein, or an active fragment of any of the foregoing.

4. The use or method of claim 3, wherein the cytokine polypeptide is IL-2, or a mutein, a variant, an active fragment, or a subunit of any of the foregoing.

5. The use or method of claim 3, wherein the cytokine polypeptide is IL-12, or a mutein, a variant, an active fragment, or a subunit of any of the foregoing.

6. The use or method of claim 3, wherein the cytokine polypeptide is interferon alpha, interferon beta, interferon gamma, or a mutein, or an active fragment of any of the foregoing.

7. The use or method of any one of the preceding claims, wherein the inducible cytokine prodrug has the formula: 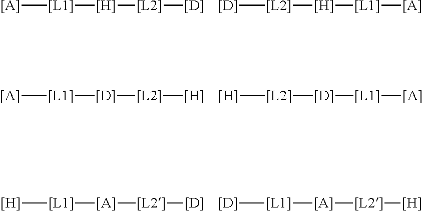
wherein A is a cytokine polypeptide, D is a blocking moiety, H is a half-life extension moiety, L1 is a protease-cleavable polypeptide linker, L2 is an polypeptide linker that is optionally protease-cleavable, and L2′ is a protease-cleavable polypeptide linker.

8. The use or method of any one of the preceding claims, wherein the inducible cytokine prodrug is a single polypeptide chain.

9. The use or method of any one of claims 1-8, wherein the inducible cytokine prodrug comprises at least two polypeptide chains.

10. The use or method of any one of claims 1-8, wherein the inducible cytokine prodrug comprises at least three polypeptide chains.

11. The use or method of any one of the preceding claims, wherein the cytokine polypeptide comprises Compound 1, Compound 2, Compound 3, Compound 4, or an amino acid sequence variant of the foregoing.

12. The use or method of claim 11, wherein a) Compound 1 comprises a first polypeptide chain of SEQ ID NO:1 and a second polypeptide chain of SEQ ID NO:5, and the amino acid sequence variant of Compound 1 comprises a first polypeptide chain that has at least about 80% identity to SEQ ID NO:1 and a second polypeptide chain that has at least about 80% identity to SEQ ID NO:5, b) Compound 2 comprises a first polypeptide chain of SEQ ID NO:2 and a second polypeptide chain of SEQ ID NO:5, and the amino acid sequence variant of Compound 2 comprises a first polypeptide chain that has at least about 80% identity to SEQ ID NO:2 and a second polypeptide chain that has at least about 80% identity to SEQ ID NO:5, C) Compound 3 comprises a first polypeptide chain of SEQ ID NO:3 and a second polypeptide chain of SEQ ID NO:5, and the amino acid sequence variant of Compound 3 comprises a first polypeptide chain that has at least about 80% identity to SEQ ID NO:3 and a second polypeptide chain that has at least about 80% identity to SEQ ID NO:5, and c) Compound 4 comprises a first polypeptide chain of SEQ ID NO:1 and a second polypeptide chain of SEQ ID NO:4, and the amino acid sequence variant of Compound 4 comprises a first polypeptide chain that has at least about 80% identity to SEQ ID NO:4 and a second polypeptide chain that has at least about 80% identity to SEQ ID NO:5.

12. The use or method of any one of claims 1-9, wherein the cytokine polypeptide is Compound 5, Compound 6, Compound 7, Compound 8, Compound 9, or Compound 10, or an amino acid sequence variant of the foregoing.

13. The use or method of claim 12, wherein a) Compound 5 comprises a first polypeptide chain of SEQ ID NO: 6 and a second polypeptide chain of SEQ ID NO:12, and the amino acid sequence variant of Compound 5 comprises a first polypeptide chain that has at least about 80% identity to SEQ ID NO: 6 and a second polypeptide chain that has at least about 80% identity to SEQ ID NO: 12, b) Compound 6 comprises a first polypeptide chain of SEQ ID NO: 7 and a second polypeptide chain of SEQ ID NO: 12, and the amino acid sequence variant of Compound 6 comprises a first polypeptide chain that has at least about 80% identity to SEQ ID NO:7 and a second polypeptide chain that has at least about 80% identity to SEQ ID NO: 12, C) Compound 7 comprises a first polypeptide chain of SEQ ID NO: 8 and a second polypeptide chain of SEQ ID NO: 13, and the amino acid sequence variant of Compound 7 comprises a first polypeptide chain that has at least about 80% identity to SEQ ID NO: 8 and a second polypeptide chain that has at least about 80% identity to SEQ ID NO: 13, c) Compound 8 comprises a first polypeptide chain of SEQ ID NO: 9 and a second polypeptide chain of SEQ ID NO: 13, and the amino acid sequence variant of Compound 8 comprises a first polypeptide chain that has at least about 80% identity to SEQ ID NO: 9 and a second polypeptide chain that has at least about 80% identity to SEQ ID NO: 13, d) Compound 9 comprises a first polypeptide chain of SEQ ID NO: 10 and a second polypeptide chain of SEQ ID NO: 13, and the amino acid sequence variant of Compound 9 comprises a first polypeptide chain that has at least about 80% identity to SEQ ID NO: 10 and a second polypeptide chain that has at least about 80% identity to SEQ ID NO: 13, and e) Compound 10 comprises a first polypeptide chain of SEQ ID NO: 11 and a second polypeptide chain of SEQ ID NO: 13, and the amino acid sequence variant of Compound 10 comprises a first polypeptide chain that has at least about 80% identity to SEQ ID NO: 11 and a second polypeptide chain that has at least about 80% identity to SEQ ID NO: 1.

14. The use or method of any one of claims 1-9, wherein the inducible cytokine prodrug comprises the amino acid selected from 14-20, or an amino acid sequence that has at least about 80% identity to SEQ ID NOs 14-20.

15. The use or method of any one of the preceding claims, wherein an anti-PD-1 antibody is administered.

16. The use or method of claim 15, wherein the anti-PD-1 antibody is selected from the group consisting of AMP-224 (AstraZenica), 609A (3SBio), 704 (3SBio), 705 (3SBio), ABBV-181 (AbbVie), ADU-1503/bion-004 (Chinook Therapeutics), AGEN2034/balstilimab (Agenus), AK103 (Akeso), AK104 (Akeso), AK112 (Akeso), AK123 (Akeso), AMG 256 (Amgen), AMG 404 (Amgen), ANB030 (AnaptysBio), ANKEBIO Anti-PD1 product (Anhui Anke Biotechnology), Anti PD-1/Anti-CD47 (DiNonA), ASKG915 (Ask Gene Pharmaceuticals), AV-MEL-1 (Aivita Biomedical), BCD-100 (Biocad CJSC), BI 754091 (Boehringer Ingelheim), BiCKI-IL-7 (OSE Immunotherapeutics), Boehringer-PD-1-unknown (Boehringer Ingelheim), BSK-050K01 (Biosion), Camrelizumab (Jiangsu Hengrui Medicine), CB201 (Crescendo Biologics), CB213 (Crescendo Biologics), CC-90006 (AnaptsBio), cetrelimab (J&J), chPD1 (Kiromic Biopharma), CMAB819 (Mabpharm), CS1003 (CStone Pharmaceuticals), CS17938 (Shenzhen Chipscreen Biosciences), CTX-8371 (Compass Therapeutics), CX-072 (CytomX Therapeutics), CX-188 (CytomX Therapeutics), cypalizumab (Harbin Gloria Pharmaceuticals), DB004 (DotBio), EMB02 (EpimAb Biotherapeutics), Geptanblimab/genolimzumab (Apollomics), GS19 (Suzhou Zelgen Biopharmaceuticals), HLX10 (Shanghai Henlius Biotech), HX008 (Taizhou HanZhong Pharmaceuticals), HY003 (Juventas Cell Therapy), IBI315/BH2950 (Innovent Biologics), IBI318 (Innovent Biologics), IBI319 (Innovent Biologics), IMM1802 (ImmuneOnco Biopharma), IMT200 (TrueBinding), Jemperli/dostarlimab (AnaptysBio), JTX-4014 (Jounce Therapeutics), Keytruda/pembrolizumab (Merck), LBL-006 (Nanjing Leads Biolabs), Libtayo/cemiplimab-rwlc (Regeneron Pharmaceuticals), LVGN3616 (Lyvgen Biopharma), LXF821 (Novartis), LY01015 (Luye Pharma Group), LY3462817 (Eli Lilly), MCLA-134 (Merus N.V.), MEDI5752 (AstraZenica), NIR178 (Novartis), ONCR-177 (Oncorus), ONO-4685 (Ono Pharmaceutical), Opdivo/nivolumab (Ono Pharmaceutical), MGD019 (MacroGenius), PD1-GDT CAR-T (Kiromic Biopharma), penpulimab (Akeso), PSB205 (Qilu Puget Sound Biotherapeutics), PT-001 (Merck), PT627 (Merck), RB-M1 (Refuge Biotechnologies), Retifanlimab (MacroGenics), RG6139 (Roche), RG6279 (Roche), RTX-002 (RubrYc Therapeutics), sasanlimab (Pfizer), Servier-PD1×LAG3-unknown (Servier), SL-279252/TAK-252 (Shattuck Labs), Sofusa anti-PD1 (Sorrento Therapeutics), spartalizumab (Novartis), SSI-361 (Lyvgen Biopharma), Sym021 (Servier), Tebotelimab (MacroGenics), tislelizumab (BeiGene), TSR-075 (AnaptsBio), Tuhura-DO/PD-1-unknown (Tuhura Biopharma), toripalimab (Shanghai Junshi Biosciences), sintilimab (Innovent Biologics), Unicar-CAR-T&PD-1-unknown (Shanghai Unicar-Therapy Bio-Medicine Technology), Xdivane (Xbrane Biopharma), XmAb20717 (Xencor), XmAb23104 (Xencor), YBL-006 (Y-Biologics), zimberelimab (Arcus Biosciences)

17. The use or method of claim 16, wherein the anti-PD-1 antibody is pembrolizumab, dostarlimab, cemiplimab-rwlc, nivolumab, camrelizumab, tislelizumab, toripalimab, sintilimab or a biosimilar of any of the foregoing.

18. The use or method of any one of claims 1-17, wherein an anti-PD-L1 antibody is administered.

19. The use or method of claim 18, wherein the anti-PD-L1 antibody is chosen from the group consisting of A167 (Sichuan Kelun), ABL501 (ABL Bio), ABL503 (ABL Bio), ABSK041 (Abbisko Therapeutics), ACE1708 (Acepodia), ACE-NK-PDL1 (Acepodia), ADG104 (Adagene), AK106 (Akeso), ALPN-202 (Alpine Immune Sciences), AN4005 (Adlai Nortye Biopharma), BMS-936559/MDX-1105 (BMS), APL-502/TQB2450 (Apollomics), Arbutus-PD-L1-unknown (Arbutus Biopharma), ASC22 (Ascletis Pharma), ATG-101 (Antengene), AVA-004 (Avacta Group), AVA021 (Avacta Group), AVA027 (Avacta Group), AVA-040-100 (Avacta Group), AVA04-Vbp (Avacta Group), Bavencio/avelumab (Merck), BCD-135 (Biocad CJSC), BGB-A333 (BeiGene), Bintrafusp alfa/GSK4045154 (Merck), CA-170/aupm-170 (Dr. Reddy's Laboratories), CCX559 (ChemoCentryx), CDR101 (CDR-Life), cosibelimab (Checkpoint Therapeutics), CTX-8371 (Compass Therapeutics), DiNonA-Solid Tumors-unknown (DiNonA), DR30207 (Zhejiang Doer Biologics), DuoBody-PD-L1×4-1BB (Ligand Pharmaceuticals), envafolimab (Alphamab Oncology), EPIM-001 (Elpis Biopharmaceuticals), ES101 (Elpiscience Biopharma), INBRX-105 (Inhibrx), FAZ053 (Novartis), FS118 (F-star Therapeutics), GB262 (Genor Biopharma), GS-4224 (Gilead), GT900008 (Kintor Pharmaceuticals), GX-P2 (Genexine), Hamni-PS-L1/CD47-Unknown (Hanmi Pharmaceutical), HBM7015 (HBM Holdings), HBM9167 (HBM Holdings), HLX20 (Shanghai Henlius Biotech), HTI-1088 (Jiangsu Hengrui Medicine), IBI318 (Innovent Biologics), IBI322 (Innovent Biologics), IBI323 (Innovent Biologics), IGM-7354 (IGM Biosciences), IMC-001 (Sorrento Therapeutics), Imfinzi/durvalumab (AstraZenica), IMM25 (ImmuneOnco Biopharma), IMM2502 (ImmuneOnco Biopharma), IMM2503 (ImmuneOnco Biopharma), IMM2504 (ImmuneOnco Biopharma), INCB86550 (Incyte), I0103 (IO Biotech), JS003 (Shanghai Junshi Biosciences), Jubilant-PD-L1-unknown (Jubilant Therapeutics), KD033 (Kadmon Holdings), KN046 (Alphamab Oncology), KY1003 (Sanofi), KY1043 (Sanofi), LY3300054 (Eli Lilly), LY3415244 (Eli Lilly), MRNA-6981 (Moderna), MSB2311 (Transcenta Holding), MT-6035 (Molecular Templates), ND021/NM21-1480 (Numab Therapeutics), OX001R (Oxford BioTherapeutics), PD-L1 based BsAbs (I-Mab), PD-L1 Boltbody ISAC (Bolt Biotherapeutics), PDL-GEX (Glycotope GmbH), PMC-122 (PharmAbcine), PMI06 (D&D Pharmatech), Protheragen-RV-scFv-PDL1-unknown (Protheragen), PRS-344 (Pieris Pharmaceuticals), Q-1802 (Merck), RC98 (Yantai Rongchang Pharmaceutical), RV-scFv-PDL1 (Protheragen), SenI_TAAx22P (Hebei Senlang Biotechnology), SHC020 (Nanjing Sanhome Pharmaceutical), sugemalimab (Ligand Pharmaceuticals), atezolizumab (Roche), TST005 (Transcenta Holding), TT-01 (Topmunnity Therapeutics), TTX-siPDL1 (TransCode Therapeutics), UniCAR-T-PD-L1 (GEMoaB monoclonals), Vaximm (VXM10), and YBL-013 (Y-Biologics).

20. The use or method of claim 19, wherein the anti-PD-1 antibody is avelumab, durvalumab, atezolizumab or a biosimilar of any of the foregoing.

21. The use or method of any one of the preceding claims, wherein the half-life extension element is a human serum albumin, an antigen binding polypeptide that binds human serum albumin, or an immunoglobulin Fc or fragment thereof.

22. The use or method of any one of the preceding claims, wherein the protease cleavable linker comprises a sequence that is capable of being cleaved by a protease selected from kallikrein, thrombin, chymase, carboxypeptidase A, cathepsin, elastase, PR-3, granzyme M, a calpain, a matrix metalloproteinase (MMP), an ADAM, a FAP, a plasminogen activator, a caspase, a tryptase, or a tumor protease.

23. The use or method of any one of the preceding claims, wherein the protease is selected from cathepsin B, cathepsin C, cathepsin D, cathepsin E, cathepsin K, cathepsin L, or cathepsin G.

24. The use or method of any one of the preceding claims, wherein protease is selected from matrix metalloprotease (MMP) is MMP1, MMP2, MMP3, MMP8, MMP9, MMP10, MMP11, MMP12, MMP13, or MMP14.

25. The use or method of any one of the preceding claims, wherein the blocking element binds the cytokine polypeptide.

26. The use or method of any one of the preceding claims, wherein the blocking element comprises a ligand-binding domain or fragment of a cognate receptor for the IFN, an antibody or antigen-binding fragment of an antibody that binds to the IFN polypeptide.

27. The use or method of any one of the preceding claims, wherein the antibody or antigen-binding fragment is a single domain antibody, a Fab, or a scFv that binds the IFN polypeptide.

28. The use or method of any one of the preceding claims, wherein the inducible cytokine prodrug is administered before, concurrently with or after the anti-PD-1 antibody or the anti-PD-L1 antibody.

29. The use or method of any one of the preceding claims, wherein the cancer is adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

30. The use or method of claim 29, wherein the cancer is colon cancer, lung cancer, melanoma, renal cell carcinoma, or breast cancer.

31. The use or method of claim 29, wherein the cancer is melanoma, non-small cell lung cancer (NSCLC), small cell lung cancer (SCLC), head and neck squamous cell cancer (HNSCC), classical Hodgkin lymphoma (cHL), primary mediastinal large B cell lymphoma (PMBCL), urothelial carcinoma, microsatellite instability high or mismatch repair deficient cancer, microsatellite instability high or mismatch repair deficient colorectal cancer, gastric cancer, esophageal cancer, cervical cancer, hepatocellular carcinoma (HCC), merkel cell carcinoma (MCC), renal cell carcinoma (RCC), endometrial carcinoma, tumor mutational burden high cancer, cutaneous squamous cell carcinoma (cSCC), triple negative breast cancer (TNBC), colorectal cancer or oesophageal carcinoma.

32. The use or method of claim 29, wherein the cancer is metastatic renal clear cell carcinoma or metastatic cutaneous malignant melanoma.

33. A pharmaceutical composition comprising the inducible cytokine prodrug, an anti-PD-1 or an anti-PD-L1 antibody, and a suitable carrier.

34. The pharmaceutical composition of claim 33, wherein the composition is a liquid composition for intravenous administration.

35. The pharmaceutical composition of claim 33, wherein the composition is a lyophilized composition.

36. The pharmaceutical composition of claim 35, wherein the lyophilized composition is for reconstitution using water for formulation is suitable of intravenous administration.

37. The use, method or composition of any one of claims 1-32, wherein the cancer is a lymphoma.

38. The use, method or composition of claim 37, wherein the lymphoma is a B cell lymphoma or a T cell lymphoma.

39. The use, method or composition of claim 37, wherein the lymphoma is Non-Hodgkin lymphoma or Hodgkin lymphoma.

40. The use, method or composition of claim 37, wherein the lymphoma is diffuse large B-cell lymphoma, primary mediastinal B cell lymphoma, follicular lymphoma, small lymphocytic lymphoma, chronic lymphocytic leukemia, marginal zone lymphoma, mantle cell lymphoma, Waldenstrom's macroglobulinemia, Burkitt lymphoma, peripheral T cell lymphoma, anaplastic large cell lymphoma, angioimmunoblastic lymphoma, cutaneous T cell lymphoma, central nervous system lymphoma, grey zone lymphoma, double hit lymphoma, triple hit lymphoma, high grade B cell lymphomas not otherwise specified, lymphoblastic lymphoma, lymphoplasmacytic lymphoma, MALT lymphoma, monocytoid B cell lymphoma, natural killer (NK) cell lymphoma, mycosis fungoides, Sezary syndrome, enteropathy-type T cell lymphoma, or hepatosplenic gamma/delta T cell lymphoma.

41. A method for treating lymphoma, comprising administering to a subject in need thereof an effective amount of an inducible cytokine prodrug.

42. An inducible cytokine prodrug for use in treating lymphoma.

43. The method or use of claim 41 or 42 wherein the inducible cytokine prodrug is an inducible IL-12 prodrug or an inducible IFNalpha prodrug.

44. The method of use of claim 41 or 42, wherein the lymphoma is a B cell lymphoma or a T cell lymphoma.

45. The method or use of claim 41 or 42, wherein the lymphoma is Non-Hodgkin lymphoma or Hodgkin lymphoma.

46. The method or use of claim 41 or 42, wherein the lymphoma is diffuse large B-cell lymphoma, primary mediastinal B cell lymphoma, follicular lymphoma, small lymphocytic lymphoma, chronic lymphocytic leukemia, marginal zone lymphoma, mantle cell lymphoma, Waldenstrom's macroglobulinemia, Burkitt lymphoma, peripheral T cell lymphoma, anaplastic large cell lymphoma, angioimmunoblastic lymphoma, cutaneous T cell lymphoma, central nervous system lymphoma, grey zone lymphoma, double hit lymphoma, triple hit lymphoma, high grade B cell lymphomas not otherwise specified, lymphoblastic lymphoma, lymphoplasmacytic lymphoma, MALT lymphoma, monocytoid B cell lymphoma, natural killer (NK) cell lymphoma, mycosis fungoides, Sezary syndrome, enteropathy-type T cell lymphoma, or hepatosplenic gamma/delta T cell lymphoma.
